SEQUENCE LISTING (7 sequences)

SEQ ID NO: 1 - DNA, 23 bp, synthetic construct
  source 1..23: mol_type = other DNA
ctttgagaccgaggtgtatcacc

SEQ ID NO: 2 - DNA, 22 bp, synthetic construct
  source 1..22: mol_type = other DNA
ggtcagcatgttgtaccacagg

SEQ ID NO: 3 - DNA, 19 bp, synthetic construct
  source 1..19: mol_type = other DNA
ccaaggaccgcaagaagat

SEQ ID NO: 4 - DNA, 20 bp, synthetic construct
  source 1..20: mol_type = other DNA
ctcctctggtgaggagtgct

SEQ ID NO: 5 - DNA, 20 bp, synthetic construct
  source 1..20: mol_type = other DNA
atgttcgtcatgggtgtgaa

SEQ ID NO: 6 - DNA, 20 bp, synthetic construct
  source 1..20: mol_type = other DNA
tgtggtcatgagtccttcca

SEQ ID NO: 7 - RNA, 23 nt, Homo sapiens
  source 1..23: mol_type = other RNA
tagcaccatttgaaatcagtgtt

(19) United States
(12) Patent Application Publication
(10) Pub. No.: US 2024/0011034 A1
(43) Pub. Date: Jan. 11, 2024

(54) INHIBITION OF miR-29b-3p TO ENHANCE NEURONAL SURVIVAL IN HUNTINGTON'S DISEASE

(71) Applicant: Washington University, St. Louis, MO (US)

(72) Inventors: Andrew Yoo, St. Louis, MO (US); Youngmi Oh, St. Louis, MO (US)

(21) Appl. No.: 18/348,263

(22) Filed: Jul. 6, 2023

Related U.S. Application Data
(60) Provisional application No. 63/358,765, filed on Jul. 6, 2022.

(51) Int. Cl. C12N 15/113; A61K 31/64; A61P 25/28

(52) U.S. Cl. CPC C12N 15/113; A61K 31/64; A61P 25/28; C12N 2310/11

(57) ABSTRACT

The present invention relates to compositions and methods for modulating miRNAs acitivity in a population of cells or a subject. More particularly, the invention relates to inhibiting the expression and/or activity of miR-29b-3p to enhance neuronal survival during neurodegeneration.

### CROSS REFERENCE

The present application claims the benefit of U.S. Provisional Patent Application No. 63/358,765, entitled, “INHIBITION OF miR-29b-3p TO ENHANCE NEURONAL SURVIVAL IN HUNTINGTON′S DISEASE” filed Jul. 6, 2022, the content of which is hereby incorporated by reference in its entirety.

### GOVERNMENT SUPPORT

This invention was made with government support under NS107488 awarded by the National Institutes of Health. The government has certain rights in the invention.

### FIELD OF THE TECHNOLOGY

The present invention relates to compositions and methods for modulating miRNAs acitivity in a population of cells or a subject. More particularly, the invention relates to inhibiting the expression and/or activity of miR-29b-3p to enhance neuronal survival during neurodegeneration.

### INCORPORATION OF SEQUENCE LISTING

The present application contains a Sequence Listing which has been submitted in .XML format via Patent Center and is hereby incorporated by reference in its entirety. Said WIPO Sequence Listing was created on Jul. 6, 2023, XML copy is named 020121_US-NP_Sequence_Listing.xml, and is 6.96 kilobytes in size.

### BACKGROUND

Huntington's disease (HD) is an inherited neurodegenerative disorder characterized by a range of symptoms, including motor deficits, psychiatric symptoms, and cognitive decline. HD pathology results from a mutation that expands the polymorphic glutamine (CAG) tract within the HTT gene to more than 36 repeats, where the majority of HD patients contain a CAG repeat size of 40-50, leading to adult-onset of clinical symptoms. The number of CAG repeats is directly linked to the severity of the disease and is inversely proportional to the age of onset. However, how aging in HD patients drives the onset of neurodegeneration remains unclear.

MicroRNAs (miRNAs) are single-stranded, non-coding RNAs that regulate transcription and translation of coding RNAs (mRNA). Since their discovery in 1993, miRNAs have emerged as key regulators in numerous physiological and pathological processes. miRNAs are highly conserved and are about 18-25 nucleotides in length. Typically, miRNAs direct translational repression by binding to the 3′ untranslated region (UTR) of mRNAs. Because only partial complementarity is required for miRNA-mRNA interactions, a single miRNA can potentially regulate hundreds of mRNA transcripts.

Therefore, there is an unmet need for identifying miRNA targets which regulate aging in HD and MSN degeneration which can offer a potential therapeutic approach to provide MSN resilience against neurodegeneration in HD.

### SUMMARY

In some aspects, the present disclosure encompasses a synthetic antisense RNA for targeting miR-29b-3p for treatment of Huntington's Disease. For instance, in some aspects, the present disclosure encompasses a composition comprising a therapeutic amount of one or more antisense RNA for targeting miR-29b-3p.

In other aspects, the present disclosure encompasses a method of treatment for Huntington's disease in a subject in need thereof. The method comprises administering an miR-29b-3p inhibitor to the subject, wherein administration results in enhanced neuronal autophagy. In particular embodiments, the miR-29b-3p inhibitor is an antisense RNA targeting miR-29b-3p.

In still other aspects, the present disclosure encompasses a method of treatment of Huntington's disease in a subject in need thereof, where the method comprises administering a glibenclamide analog to the subject, wherein administration results in enhanced neuronal autophagy. In particular embodiments, the glibbenclamide analog is G2-115.

Other aspects and iterations of the disclosure are detailed below.

### BRIEF DESCRIPTION OF THE FIGURES

FIG. 1A-1E show differential manifestation of neurodegeneration between MSNs derived from healthy control individuals and HD patients. FIG. 1A shows the experimental scheme for MSN derivation from fibroblasts of pre-symptomatic (Pre-HD-MSNs), symptomatic HD patients (HD-MSNs), and their respective healthy control individuals of similar ages (Young/Old-MSNs). FIG. 1B shows representative images of TUBB3-, DARPP32-, and MAP2-positive cells from Young/Old healthy control MSNs, Pre-HD-MSNs, and HD-MSNs. Scale bars are 20 μm, FIG. 1C Quantification of TUBB3-, MAP2- and DARPP-32-positive cells in FIG. 1B; averages of 300-600 cells per group from independent HD and healthy control lines. Each dot represents one individual's reprogrammed MSN. FIG. 1D shows the average long gene expression (LGE), a transcriptomic feature of neuronal identity, in fibroblasts, young/old-MSNs, Pre-HD-, and HD-MSNs. X-axis corresponds to gene lengths (kb) defined by the start and stop genomic coordinates and Y-axis corresponds to gene expression (CPM). The dotted line depicts 100 kb in gene length. FIG. 1E shows representative images (left) and quantification (right) of SYTOX-positive cells as a fraction of Hoechst-positive cells. Each dot represents one individual's reprogrammed MSN. Scale bars in are 100 μm. Statistical significance was determined by one-way ANOVA. ****p<0.0001, ***p<0.001, ns, not significant. Mean±s.e.m.

FIG. 2A-2E show healthy control and HD patient fibroblasts can be directly reprogrammed into MSNs. FIG. 2A shows reprogrammed cells by transduction of miR-9/9*-124+CDM immunostained for neuronal markers, TUBB3, and MSN marker, DARPP32 at PID30. Scale bar is 20 μm. FIG. 2B shows expression of each CDM factor in reprogramming cells by immunostaining for CTIP2, DLX1, DLX2, MYT1L, and TUBB3. Non-transduced fibroblasts were used as a negative control for immunostaining. Scale bar is 20 μm. FIG. 2C shows the neuronal morphology across all samples used in the examples stained positive for TUBB3 successfully undergo direct conversion by miR-9/9*-124+CDM. Scale bars 20 μm. FIG. 2D shows representative images of healthy control Young, Old, Pre-HD, and HD MSNs marked by TUBB3 in reprogrammed Healthy control Young, Old, Pre-HD, and HD-MSNs at PID21 and PID35. FIG. 2E shows the measurement of mean neurite length and mean number of neurite branches in reprogrammed Healthy control Young, Old, Pre-HD, and HD-MSNs at PID21 and PID35. Scale bar is 100 μm. Images processed by CellProfiler to identify neurites and associated cell soma. Each dot represents one individual's reprogrammed MSN. Statistical significance was determined using one-way ANOVA, ****p<0.0001, ***p<0.001, **p<0.01, ns, not significant. Mean±s.e.m.

FIG. 3A-3I show genetic networks altered in HD-MSNs by WGCNA. FIG. 3A shows the signed association of protein-coding genes with Age, Symptomatic onset, and Sex condition of Huntington's disease. Modules with positive values indicate increased expression in HD-MSNs; modules with negative values indicate decreased expression in HD-MSNs. The red dotted lines indicate correlation values of 0.7 or −0.7 with p=10-7 for age, p=10-6 for symptomatic onset, and p=10-6 for sex. FIG. 3B shows an expression heatmap of our selected modules of HD samples (blue, lightcyan1, brown, and greenyellow) from WGCNA. FIG. 3C shows pathways enriched in downregulated (log2FC<−1) or upregulated (log2FC>1) genes in the greenyellow, blue and lightcyan1 module of Huntington's disease by BioPlanet analysis. FIG. 3D shows the signed association of protein-coding genes with age and sex of healthy control. Modules with positive values indicate increased expression in Old-MSNs; modules with negative values indicate decreased expression in Old-MSNs. The red dotted lines indicate correlation values of 0.7 or −0.7 with p=10-7 for age and p=10-6 for sex condition. FIG. 3E shows pathways enriched in upregulated (log2FC>1) genes in the honeydewl module of healthy control by BioPlanet analysis. FIG. 3F shows summary preservation statistics as a function of the module size. The composite preservation statistic (Zsummary) (left), the connectivity statistics (Zconnectivity) (middle), the density statistics (Zdensity) (right). Each point represents a module, labeled by color. The dashed blue and green lines indicate the thresholds Z=2 and Z=10, respectively. FIG. 3G shows Representative images of MSNs expressing the tandem monomeric mCherry-GFP-LC3 reporter immunostained for TUBB3. Autophagosome (i.e., mCherry+, GFP+ puncta) and autolysosome (i.e., mCherry+, GFP− puncta) compartments in MSNs from HD patients and control individual. Scale bar 20 μm. FIG. 3H shows reprogrammed cells immunostained for p62 and TUBB3 from independent HD and healthy control lines. Scale bar 20 μm. FIG. 3I Immunoblot analysis for p62 and GAPDH of three independent pre-HD- and three independent HD-MSN lines at PID26. P62 signal intensities were normalized by GAPDH signals and relative fold changes in HD-MSNs were calculated over pre-HD-MSNs.

FIG. 4A-4I show identification of gene module associated with autophagy dysfunction in HD-MSNs by weighted gene coexpression network analysis (WGCNA). FIG. 4A shows a heatmap of six gene modules associated with age and disease stage of HD (left). Pathways enriched in downregulated genes (log2FC<−1) in the brown module by BioPlanet analysis (right). FIG. 4B shows a heatmap of six modules associated with age in Ctrl-MSNs from young and old healthy individuals (left). Pathways enriched in downregulated genes (log2FC<−1) in the lavenderblush3 module by BioPlanet analysis (right). FIG. 4C shows Venn diagram of the number of gene members from the brown module either overlapping or distinct from gene members in the lavenderblush3 module from control groups. The diagram also contains several representative genes. FIG. 4D shows Protein-Protein interaction network of the brown module. FIG. 4E shows chemical compounds predicted as upstream regulators of the brown module by Ingenuity Pathway Analysis (IPA). FIG. 4F shows representative images of MSNs expressing the tandem monomeric mCherry-GFP-LC3 reporter (left), and quantification of autophagosome (i.e., mCherry+, GFP+ puncta) and autolysosome (i.e., mCherry+, GFP− puncta) compartments in MSNs from multiple HD patients and control individuals. Measurements were performed in cells having at least 3 puncta per cell (from more than 50 cells per MSN line) (right). Each dot represents one individual's reprogrammed MSNs. Scale bar is 10 μm. FIG. 4G shows representative images (left) and quantification of CYTO-ID green signals in MSNs from multiple HD patients and control individuals (right) at PID 26. Each dot represents one individual's reprogrammed MSN. Scale bar is 20 μm. FIG. 4H shows reprogrammed cells immunostained for p62 and TUBB3 (right) and quantification of p62 intensity per cell in MSNs from independent HD and healthy control lines. Each dot represents one individual's reprogrammed MSN. Scale bar is 10 μm. FIG. 4I shows representative images (top) and quantification (bottom) of neuronal apoptosis in pre-HD-MSNs and HD-MSNs (four pre-HD-MSN lines and three HD-MSN lines). The green signal represents Caspase 3/7 activation (PID 26) and Annexin V signal (PID 30). Each dot represents one individual's reprogrammed MSNs. Scale bar is 100 μm. Statistical significance was determined by one-way ANOVA (FIG. 4F-4H) and unpaired t-test (FIG. 4I). ****p<0.0001, ***p<0.001, **p<0.01, *p<0.05. Mean±s.e.m.

FIG. 5A-5K show autophagy inhibitor induces neurodegeneration of pre-HD-MSNs. FIG. 5A shows representative images of p62 and TUBB3-positive cells from multiple healthy control young and pre-HD-MSNs treated with DMSO or 50 μM LY294002. Scale bar is 20 μm. FIG. 5B shows quantification of p62 intensity per cell at PID26 from multiple healthy control young and pre-HD-MSNs treated with DMSO or 50 μM LY294002. FIG. 5C shows representative images of CYTO-ID green signals from multiple healthy control young and pre-HD-MSNs treated with DMSO or 50 μM LY294002. Scale bar is 20 μm. FIG. 5D shows quantification of CYTO-ID signals per cell at PID26 from multiple healthy control young and pre-HD-MSNs treated with DMSO or 50 μM LY294002. FIG. 5E shows representative images of SYTOX staining from multiple healthy control young and pre-HD-MSNs treated with DMSO or 50 μM LY294002. Scale bar is 100 μm. FIG. 5F shows quantification SYTOX-positive cells as a fraction of Hoechst-positive cells at PID30 from multiple healthy control young and pre-HD-MSNs treated with DMSO or 50 μM LY294002. FIG. 5G shows representative images of caspase activation (green) at PID 26 from three pre-HD-MSN lines treated with DMSO or 50 μM LY294002. Scale bar is 100 μm. FIG. 5H shows quantification of caspase activation at PID 26 from three pre-HD-MSN lines treated with DMSO or 50μM LY294002. FIG. 5I shows representative images of Annexin V signal (green) at PID 30 from three pre-HD-MSN lines treated with DMSO or 50 μM LY294002. Scale bar is 100 μm. FIG. 5J shows quantification of Annexin V signal at PID 30 from three pre-HD-MSN lines treated with DMSO or 50 μM LY294002 (e) from three pre-HD-MSN lines treated with DMSO or 50 μM LY294002. FIG. 5K shows representative images of HTT inclusion bodies (IBs) present in pre-HD-MSNs treated with DMSO or 50 μM LY294002 (four pre-HD-MSNs lines; DMSO: 3947 cells, 50 μM LY294002: 4314 cells, PID30). Scale bar is 20 μm. FIG. 5L shows quantification of HTT inclusion bodies (IBs) present in pre-HD-MSNs treated with DMSO or 50 μM LY294002 (four pre-HD-MSNs lines; DMSO: 3947 cells, 50 μM LY294002: 4314 cells, PID30). For all figures shown, each dot represents one individual's reprogrammed MSNs and statistical significance was determined using unpaired t-test; ****p<0.0001, ***p<0.001, **p<0.01, *p<0.05. Mean±s.e.m.

FIG. 6A-6D show the treatment of autophagy inhibitor or inducer in reprogrammed MSNs. FIG. 6A shows immunoblot of p62 and GAPDH in pre-HD-MSNs treated with DMSO or 50 μM LY294002 and HD-MSNs treated with DMSO or 0.5 μM G2-115 at PID26. p62 Intensity values were normalized by GAPDH intensities and the relative fold change over DMSO condition was calculated from immunoblot images of three independent Pre-HD-MSNs and HD-MSNs. Each dot represents one individual's reprogrammed MSN. FIG. 6B shows representative images of pre-HD-MSNs treated with DMSO or 50 μM LY294002 and HD-MSNs treated with DMSO or 0.5 uM G2-115 (PID 30) (left) and quantification of autophagosome (i.e., mCherry+, GFP+ puncta) and autolysosome (i.e., mCherry+, GFP− puncta) compartments from three independent Pre-HD-MSNs and HD-MSNs (right). MSNs express the tandem monomeric RFP-GFP-LC3 reporter. Scale bars 20 μm. Measurements were performed in cells having at least 3 puncta per cell (from more than 50 cells per MSN line). Each dot represents one individual's reprogrammed MSNs. FIG. 6C shows a schematic of synthetic route for the preparation of G2-115. FIG. 6D shows measurement of neuronal cell death of HD.40-MSNs with the treatment of DMSO or three different concentrations of G2 compound at PID 30 by Caspase-3/7 reagents (left) or Annexin V reagents (right) (n=4-8 biological replicates for each). For all figures shown, statistical significance was determined using unpaired t-test (FIG. 6A-6B) and one-way ANOVA (FIG. 6D). ****p<0.0001, ***p<0.001, *p<0.05. Mean±s.e.m.

FIG. 7A-7H show autophagy activator rescues HD-MSNs from degeneration. FIG. 7A shows chemical structure of G2-115. FIG. 7B shows representatives immunoblot (top) of steady-state levels of α1-antitrypsin Z variant (ATZ) and β-actin in the HTO/Z cell line model of α1-antitrypsin deficiency after 24 hr treatment with DMSO alone or G2-115 in DMSO at 0.5 and 2.5 μM. The normalized intensity (bottom) was calculated from immunoblot images for 0.5 μM versus DMSO control (Individual data plotted, n=4; *p=0.003 by t-test; Mean±s.e.m.). FIG. 7C show representative images of p62 and TUBB3-positive cells (top) and quantification of p62 intensity per cell (bottom) at PID26 from multiple healthy control old and HD-MSNs treated with DMSO or 0.5 μM G2-115. Each dot represents one individual's reprogrammed MSN. Scale bars is 20 μm FIG. 7D show representative images of CYTO-ID staining (top) and quantification of CYTO-ID green signals (bottom) at PID26 from multiple healthy control old and HD-MSNs treated with DMSO or 0.5 μM G2-115. Each dot represents one individual's reprogrammed MSN. Scale bar is 20 μm FIG. 7E show representative images (top) and quantification (bottom) of SYTOX-positive cells as a fraction of Hoechst-positive cells in HD-MSN treated with DMSO or different concentration of G2-115 (0.125, 0.25, or 0.5 μM) (n=4). Scale bar is 20 μm. FIG. 7F show quantification of SYTOX-positive cells from multiple healthy control old and HD-MSNs treated with DMSO or 0.5 μM G2-115. Each dot represents one individual's reprogrammed MSN. FIG. 7G show Representative images (left) and quantification (right) of caspase3/7 activation (green) and Annexin V signal (green) from three independent HD-MSN lines treated with DMSO or 0.5 μM G2-115. Each dotrepresents one individual's reprogrammed MSNs. Scale bar is 100 μm FIG. 7H show representative images (top) and quantification (bottom) of HTT inclusion bodies from three independent HD-MSNs treated with DMSO or 0.5 μM G2-115 (DMSO: 285 cells, 0.5 μM G2-115: 325 cells) at PID30. Each dot represents one individual's reprogrammed HD-MSN. Scale bar is 20 μm For all figures shown, statistical significance was determined using unpaired t-test; ****p<0.0001, ***p<0.001, **p<0.01, *p<0.05, ns, not significant. Mean±s.e.m.

FIG. 8A-8J show comparative analysis of chromatin accessibility between pre-HD- and HD-MSNs. FIG. 8A shows heatmaps showing signal intensities of ATAC-seq peaks for DARs detected between pre-HD-MSNs and HD-MSNs at PID21 (adj. p<0.05, |log2FC|>1). FIG. 8B shows pathway enrichment for genes associated with DARs at promoter regions in pre-HD-MSNs and HD-MSNs at PID21 (adj. p<0.05) by BioPlanet analysis. FIG. 8C shows integrative Genomics Viewer (IGV) snapshots showing DAR peaks (blue highlight) more accessible in pre-HD-MSNs (purple) compared to HD-MSNs (green). ATG16L1 and ATG10 are shown as these genes are involved in senescence and autophagy. FIG. 8D shows heatmap representation of ATAC-seq signal intensities for promoter regions of miRNA precursors opened in HD-MSNs. Target enrichment score (log(p-value)) of miRNA precursors for the brown module. FIG. 8E shows Integrative Genomics Viewer (IGV) snapshots showing peaks enriched in HD-MSNs (green) over pre-HD-MSNs (purple) within miR29B1 (DAR highlighted in blue). FIG. 8F shows RT-qPCR analysis of mature miR-29b-3p expression in three independent pre-HD- and HD-MSNs at PID 26. Each dot represents one individual's reprogrammed HD-MSN. FIG. 8G shows RT-qPCR analysis of mature miR-29b-3p expression in four independent healthy control young and old Ctrl-MSNs at PID 26. Each dot represents one individual's reprogrammed HD-MSN. FIG. 8H shows fold changes of increase of miR-29-3p levels in old versus young Ctrl-MSNs, and HD-MSNs versus pre-HD-MSNs, replotted from FIG. 8F and FIG. 8G. Each dot represents one individual's reprogrammed MSN. FIG. 8I shows RT-qPCR analysis of mature miR-29b-3p expression in human young striatum aged 8, 9, 11, and 19 years and human old striatum aged 83, 84, 85, 87, and 91 years. Each dot represents one individual's striatum. FIG. 8J shows RT-qPCR analysis of mature miR-29b-3p expression in human healthy control striatum (53, 71, 56, 89, and 66 years of age) and human Huntington's disease patient's striatum (59, 67, 48, 71 and 65 years of age). Each dot represents one individual's striatum. For all figures shown, statistical significance was determined using unpaired t-test; ***p<0.001, **p<0.01, *p<0.05. Mean±s.e.m.

FIG. 9A-9G show pre-HD-MSNs and HD-MSNs display differential chromatin accessibilities. FIG. 9A shows a heatmaps showing gene expression levels for DEGs that positively correlated with signal intensities of ATAC-seq in their promoter regions. Signal intensity is based on normalized CPM values. Data are shown as Z-score normalized log2CPM (adjusted p<0.05, |log2FC|>1). FIG. 9B shows GO terms associated with opened and upregulated genes. FIG. 9C shows GO terms associated with closed and downregulated genes. FIG. 9D shows transcription regulators predicted as upstream regulators of the brown module (Huntington's disease) and the lavenderblush3 (healthy control) by Ingenuity Pathway Analysis (IPA). FIG. 9E shows mature microRNAs predicted as an upstream regulator of the brown module (Huntington's disease) and the lavenderblush3 (healthy control) by Ingenuity Pathway Analysis (IPA). FIG. 9F shows microRNAs predicted as an upstream regulator of the brown module (Huntington's disease) by miRTarBase and TargetScan. FIG. 9G shows pathways enriched in target genes of miR-29b-3p in the brown module (Huntington's disease) by BioPlanet analysis.

FIG. 10A-10C show miR-29b-3p expression changes by miR-29-3p inhibitor or miR-29b overexpression lentivirus transduction. FIG. 10A shows RT-qPCR analysis of mature miR-29-3p expression levels in four HD-MSNs with control or miR-29b-3p inhibitor at PID26. Each dot represents one individual's reprogrammed HD-MSN. (***p<0.001 by unpaired t-test; Mean±s.e.m.). FIG. 10B shows Pre-HD-MSNs expressing RFP only or RFP-miR-29b immunostained with RFP and DAPI. (Scale bars, FIG. 10C shows RT-qPCR analysis of mature miR-29-3p expression levels in three independent pre-HD-MSNs expressing control or miR-29b. Each dot represents one individual's reprogrammed MSNs. (**p<0.01 by unpaired t-test; Mean±s.e.m).

FIG. 11A-11F show inhibition of miR-29b-3p enhances autophagy and rescues HD-MSNs from degeneration. FIG. 11A shows representative images (top) and quantification (bottom) of CYTO-ID green signals from pre-HD-MSNs expressing control or miR-29b (control: 353 cells, miR-29b: 392 cells) and HD-MSNs with control or miR-29b-3p inhibitor (control: 380 cells, miR-29b-3p inhibitor: 421 cells) at PID26. Scale bar is 20 μm. FIG. 11B shows representative images (top) of HD-MSNs expressing the tandem monomeric mCherry-GFP-LC3 reporter with control or miR-29b-3p inhibitor. Quantification (bottom) of autophagosome (i.e., mCherry+, GFP+ puncta) and autolysosome (i.e., mCherry+, GFP− puncta) compartments from four independent HD-MSNs. Measurements were performed in cells having at least 3 puncta per cell (from more than 50 cells per MSN line). Scale bar is 10 μm. FIG. 11C shows representative images (top) and quantification (bottom) of caspase3/7 activation at PID 26 and Annexin V signal at PID 30 from three independent pre-HD-MSN lines expressing control or miR-29b. Scale bar is 100 μm. FIG. 11D shows representative images (left) and quantification (right) of caspase3/7 activation at PID 26 and Annexin V signal at PID 30 from three independent HD-MSN lines with control or miR-29b-3p inhibitor. Scale bar is 100 μm. FIG. 11E shows representative images (left) and quantification (right) of HTT inclusion bodies from three independent pre-HD-MSNs expressing control or miR-29b (control: 437 cells, miR-29b: 389 cells). Scale bar is 20 μm. FIG. 11F shows representative images (left) and quantification (right) of HTT inclusion bodies from three independent HD-MSNs with control or miR-29b-3p inhibitor (control: 406 cells, miR-29b-3p inhibitor: 630 cells) at PID30. Scale bar is 20 μm. For all figures shown, each dot represents one individual's reprogrammed pre-HD- or HD-MSN, and statistical significance was determined using unpaired t-test; ***p<0.001, **p<0.01, *p<0.05. Scale bars in b 10 μm; in a, e, f 20 μm; in c, d 100 μm. Mean±s.e.m.

FIG. 12A-12F show the autophagy-related genes regulated by STAT3 in HD-MSNs. FIG. 12A shows the target genes of miR-29b-3p functionally related to autophagy in the brown module. Visualized by NetworkAnalyst. FIG. 12B shows a heatmap of representation of ATAC-seq signal intensities for autophagy-related genes that contained STAT3 binding site in the closed DARs in HD-MSNs (n=8-9 per group). FIG. 12C shows percentage of decrease of STAT3 levels in old versus young Ctrl-MSNs, and HD-MSNs versus pre-HD-MSNs, replotted from FIG. 13F. Each dot represents one individual's reprogrammed MSNs. (****p<0.0001 by t-test; Mean±s.e.m.). FIG. 12D shows RT-qPCR analysis of STAT3 mRNA levels in three independent pre-HD-MSN lines with shControl or shSTAT3 at PID26. Each dot represents one individual's reprogrammed Pre-HD-MSNs. (****p<0.0001 by t-test; Mean±s.e.m.). FIG. 12E shows western blot for STAT3 expression in human adult fibroblasts with shControl or shSTAT3. FIG. 12F shows RT-qPCR analysis of STAT3 mRNA levels in three independent HD-MSN lines with Control or STAT3 overexpression at PID26. Each dot represents one individual's reprogrammed HD-MSNs. (*p<0.05 by t-test; Mean±s.e.m.).

FIG. 13A-13K show inhibition of miR-29b-3p-STAT3 axis enhances autophagy and rescues HD-MSNs from degeneration. FIG. 13A shows heatmaps of STAT3 binding site intensity within chromatin loci closed in HD-MSNs. Legend depicts representative motifs for STAT3 binding sites. FIG. 13B shows Integrative Genomics Viewer (IGV) snapshots showing DAR peaks (blue highlight) more accessible in pre-HD-MSNs (purple) compared to HD-MSNs (green). ATG5 and ATG7 contained the STAT3 binding motif. FIG. 13C shows RT-qPCR analysis of ATG5 and ATG7 mRNA levels in three independent pre-HD-MSNs expressing shControl or shSTAT3 at PID26. Each dot represents one individual's reprogrammed MSN. FIG. 13D shows the sequence of miR-29-3p seeds in human and mouse STAT3 3′UTR and human STAT3 3′UTR mutant (top) and luciferase assays with HEK293Le cells co-transfected miR-NS or miR-29b-3p and wild-type or mutant of STAT3 3′UTRs containing point mutations to the seed-match regions of miR-29b-3p (bottom). The sample size (n) corresponds to the number of biological replicates (n=4). FIG. 13E shows RT-qPCR analysis of STAT3 expression in three independent pre-HD-MSNs expressing control or miR-29b (left) and four independent HD-MSNs expressing negative control or miR-29-3p inhibitor (right) at PID26. FIG. 13F shows RT-qPCR analysis of STAT3 expression in four independent healthy control young- and old-MSNs, six independent pre-HD- and HD-MSNs at PID21. FIG. 13G shows representative images (top) and quantification (bottom) of Cyto-ID green signals from three independent pre-HD-MSNs expressing shControl or shSTAT3 (for Cyto-ID, shControl: 498 cells shSTAT3: 404 cells. Scale bar is 20 μm. FIG. 13H shows representative images (top) and quantification (bottom) of HTT inclusion bodies (IBs) from three independent pre-HD-MSNs expressing shControl or shSTAT3 (control: 260 cells, miR-29b: 346 cells). Scale bar is 20 μm. FIG. 13I shows representative images (top) and quantification (bottom) of caspase3/7 activation (green) at PID 26 and Annexin V signal (green or red) at PID 30 from three independent pre-HD-MSN lines expressing shControl or shSTAT3. Scale bar is 100 μm. FIG. 13J shows representative images (top) and quantification (bottom) of caspase3/7 activation (green) at PID 26 and Annexin V signal (green or red) at PID 30 from three independent HD-MSN lines expressing control or STAT3 cDNA. Scale bar is 100 μm. FIG. 13K shows Representative images (top) and quantification (bottom) of caspase3/7 activation (green) at PID 26 and Annexin V signal (red) at PID 30 from four independent HD-MSN lines expressing control, miR-29b-3p inhibitor or shSTAT3. Scale bar is 100 μm. Each dot represents one individual's reprogrammed MSN in FIG. 13E-13K. For all figures shown, statistical significance was determined using one-way ANOVA (FIG. 13K) and unpaired t-test (FIG. 13C-13J). ****p<0.0001,***p<0.001, **p<0.01, *p<0.05, ns, not significant. Mean±s.e.m.

FIG. 14A shows predicted transcription binding sites for the DAR proximal to miR29B1 (chr7:130,878,800-130,879,437). Image from UCSC Genome Browser on Human (GRCh38/hg38). JASPAR CORE 2022, Minimum Score: 500. FIG. 14B shows quantification of Sytox-positive cells, CYTO-ID signal, STAT3 expression, and miR-29b-3p expression in fibroblasts from healthy control Young/Old, Pre-HD, and HD. Each dot represents one individual fibroblast. Statistical significance was determined by one-way ANOVA. ns, not significant.

FIG. 15A-15C show unprocessed original images of immunoblots. FIG. 15A shows unprocessed image of blots in FIG. 3I. FIG. 15B-15C show unprocessed image of blots in FIG. 6A.

### DETAILED DESCRIPTION

The present disclosure is based, in part, on the discovery that miR-29b-3p is drastically upregulated in medium spiny neurons (reprogrammed from fibroblasts of Huntington's disease (HD) patients) and basal ganglia from symptomatic patients, compared to pre-symptomatic patients and healthy individuals. An age-related correlation was seen with greater upregulation of miR-29b-3p in older HD patients compared to younger pre-symptomatic HD patients. Mechanistically, increase in miR-29b-3p was found to be correlated to enhanced apoptosis and reduced autophagy, mediated by a reduction in STAT3.

The present disclosure provides data that inhibition of miR-29b-3p is a viable treatment approach to promoting neuronal survival in Huntington's disease. Inhibition can be achieved by siRNA directly targeting miR-29b-3p or by using various pathway inhibitors like glibenclamide and novel analogs described herein.

Various aspects of the invention are described in further detail in the following sections.

### I. COMPOSITIONS

A composition of the present disclosure may comprise an inhibitor of miR-29b-3p, glibenclamide, a glibenclamide analog, or any combination thereof.

### (A) Inhibitors of miR-29b-3p

The present disclosure may comprise an inhibitor of miR-29b-3p. In some embodiments, such an inhibitor is an antisense oligonucleotide (oligo) complementary to miR-29b-3p.

In certain embodiments, an antisense oligonucleotide (oligo) comprises 8-25 nucleotides, and has at least 90% complementary to miR-29b-3p. For instance, an antisense oligo may comprise 8-25 nucleotides and be at least 90% complementary to the sequence UAGCACCAUUUGAAAUCAGUGUU (SEQ ID NO:7). In some embodiments, an antisense oligo may comprise 8, 9, 10, 11, 12, 13, 14, 15, 16, 17, 18, 19, 20, 21, 22, 23, 24, 25, or more than 25 nucleotides. In each of these embodiments, the antisense oligo may be 90, 91, 92, 93, 94, 95, 96, 97, 98, or 99% complementary to miR-29b-3p. In some embodiments, the antisense oligo may be 90, 91, 92, 93, 94, 95, 96, 97, 98, or 99% complementary to SEQ ID NO:7.

An antisense oligonucleotide of the invention may be synthesized using chemical synthesis and enzymatic ligation reactions using procedures known in the art. For example, an oligonucleotide (e.g., an antisense oligonucleotide) may be chemically synthesized using naturally occurring ribonucleotides, deoxyribonucleotides, variously modified nucleotides designed to increase the biological stability of the molecules or to increase the physical stability of the duplex formed between the antisense and sense nucleic acids, or combinations thereof. For example, phosphorothioate derivatives and acridine substituted nucleotides may be used. Other examples of modified nucleotides which may be used to generate an antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylam inomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-aino-3-N-2-carboxypropyl) uracil, (acp3)w, and 2,6-diaminopurine. Alternatively, the oligonucleotide may be produced biologically using an expression vector into which a nucleic acid has been subcloned in an antisense orientation.

In certain embodiments, antisense oligonucleotides provided herein may include one or more modifications to a nucleobase, sugar, and/or internucleoside linkage, and as such is a modified oligonucleotide. A modified nucleobase, sugar, or internucleoside linkage may be selected over an unmodified form because of desirable properties such as, for example, enhanced cellular uptake, enhanced affinity for other oligonucleotides or nucleic acid targets, and increased stability in the presence of nucleases. In certain embodiments, a modified nucleoside is a sugar-modified nucleoside. In certain such embodiments, sugar-modified nucleosides may further comprise a natural or modified heterocyclic base moiety or natural or modified internucleoside linkage and may include further modifications independent from the sugar modification. In certain embodiments, a sugar modified nucleoside is a 2′-modified nucleoside, wherein the sugar ring is modified at the 2′ carbon from natural ribose or 2′-deoxy-ribose. In certain embodiments, a 2′-modified nucleoside comprises a 2′-substituent group selected from F, O—CH₃, and OCH₂CH₂OCH₃. In certain embodiments, a 2′-modified nucleoside has a bicyclic sugar moiety. In certain embodiments, a bicyclic sugar moiety comprises a bridge group between the 2′ and the 4′ carbon atoms.

In certain embodiments, a modified oligonucleotide comprises one or more internucleoside modifications. In certain such embodiments, each internucleoside linkage of an oligonucleotide is a modified internucleoside linkage. In certain embodiments, a modified internucleoside linkage comprises a phosphorus atom.

In certain embodiments, a modified oligonucleotide comprises at least one phosphorothioate internucleoside linkage. In preferred embodiments, each internucleoside linkage of a modified oligonucleotide is a phosphorothioate internucleoside linkage.

In certain embodiments, a modified oligonucleotide comprises one or more modified nucleobases. In certain embodiments, a modified oligonucleotide comprises one or more 5-methylcytosines. In certain embodiments, each cytosine of a modified oligonucleotide comprises a 5-methylcytosine.

In certain embodiments, a modified nucleobase is selected from 5-hydroxymethyl cytosine, 7-deazaguanine and 7-deazaadenine. In certain embodiments, a modified nucleobase is selected from 7-deaza-adenine, 7-deazaguanosine, 2-aminopyridine and 2-pyridone.

In some embodiments, the antisense molecules of the invention may be modified at the base moiety, sugar moiety or phosphate backbone to improve, e.g., the stability, hybridization, or solubility of the molecule. By way of another example, the deoxyribose phosphate backbone of the nucleic acids may be modified to generate peptide nucleic acids (see Hyrup et al. (1996) Bioorganic & Medicinal Chemistry 4(I):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of a PNA has been shown to allow for specific hybridization to DNA and RNA under conditions of low ionic strength. The synthesis of PNA oligomers may be performed using standard solid phase peptide synthesis protocols as described in Hyrup et al. (1996) supra; Perry-O'Keefe et al. (1996) Proc. Natl. Acad. Sci. USA 93:14670-675.

PNAs of miR-29b-3p may be used for therapeutic and diagnostic applications. PNAs of miR-29b-3p may also be used in the analysis of single base pair mutations in a gene by PNA-directed PCR clamping; as artificial restriction enzymes when used in combination with other enzymes, such as S1 nucleases (Hyrup (1996) supra); or as probes or primers for DNA sequence and hybridization (Hyrup (1996) supra; Perry-O'Keefe et al. (1996) Proc. Natl. Acad. Sci. USA 93: 14670-675).

In other embodiments, the oligonucleotides of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides may be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

In certain embodiments, an antisense oligonucleotide of the invention is synthesized with a full phosphorothioate backbone with alternating blocks of 2′-MOE and 2′fluoro sugar-modified nucleosides.

### (B) Glibenclamide and Glibenclamide Analogs

In some embodiments, a composition of the present disclosure may comprise glibenclamide or a glibenclamide analog. Generally speaking, a glibenclamide analog has a amidoethylbenzenesulfonylurea backbone. In some embodiments, the glibenclamide analog is G2-115.

Methods of making glibenclamide, and glibenclamide analogs, are known in the art.

### (C) Pharmaceutical Formulations

Compositions detailed herein may be incorporated into pharmaceutical formulations suitable for administration. Such formulations typically comprise a component detailed in sections (A) and (B) above, or a combination thereof, and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds may also be incorporated into the compositions.

The invention includes methods for preparing pharmaceutical compositions for modulating the expression or activity of miR-29b-3p. Such methods comprise formulating a pharmaceutically acceptable carrier with an agent which modulates expression or activity of miR-29b-3p. Such compositions can further include additional active agents. Thus, the invention further includes methods for preparing a pharmaceutical composition by formulating a pharmaceutically acceptable carrier with an agent which modulates expression or activity of miR-29b-3p and one or more additional active compounds.

An agent which modulates expression or activity may, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the knowledge of the ordinarily skilled artisan. The dose(s) of the small molecule will vary, for example, depending upon the identity, size, and condition of the subject or sample being treated, further depending upon the route by which the composition is to be administered, if applicable, and the effect which the practitioner desires the small molecule to have. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of miR-155, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention may be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.), or phosphate buffered saline (PBS). In all cases, a composition may be sterile and may be fluid to the extent that easy syringeability exists. A composition may be stable under the conditions of manufacture and storage and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally may include an inert diluent or an edible carrier. Oral compositions may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents and/or adjuvant materials may be included as part of the composition. The tablets, pills, capsules, troches, and the like, may contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressured container or dispenser which contains a suitable propellant, e.g., a gas such as carbon dioxide, or a nebulizer.

Systemic administration may also be by transmucosal or transdermal means. For transmucosal or transdermal administration, penetrants appropriate to the barrier to be permeated are used in the formulation. Such penetrants are generally known in the art, and may include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds may also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. These may be prepared according to methods known to those skilled in the art, for example, as described in U.S. Pat. No. 4,522,811.

The nucleic acid molecules of the invention may be inserted into vectors and used as gene therapy vectors. Gene therapy vectors may be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector may include the gene therapy vector in an acceptable diluent, or can comprise a slow release matrix in which the gene delivery vehicle is imbedded.

The gene therapy vectors of the invention may be either viral or non-viral. Examples of plasmid-based, non-viral vectors are discussed in Huang et al. (1999) Nonviral Vectors for Gene Therapy. A modified plasm id is one example of a non-viral gene delivery system. Peptides, proteins (including antibodies), and oligonucleotides may be stably conjugated to plasm id DNA by methods that do not interfere with the transcriptional activity of the plasm id (Zelphati et al. (2000) BioTechniques 28:304-315). The attachment of proteins and/or oligonucleotides may influence the delivery and trafficking of the plasm id and thus render it a more effective pharmaceutical composition.

As used herein, the term “biological sample” refers to a sample obtained from a subject. Any biological sample comprising a miRNA of the invention is suitable. Non-limiting examples include blood, plasma, serum, urine, cerebrospinal fluid (CSF) and interstitial fluid (ISF). In a specific embodiment, the biological sample is selected from the group consisting of CSF, serum and urine. In another specific embodiment, the biological sample is CSF. In a specific embodiment, the biological sample comprises motor neurons. The sample may be used “as is”, the cellular components may be isolated from the sample, or a protein fraction may be isolated from the sample using standard techniques.

As will be appreciated by a skilled artisan, the method of collecting a biological sample can and will vary depending upon the nature of the biological sample and the type of analysis to be performed. Any of a variety of methods generally known in the art may be utilized to collect a biological sample. Generally speaking, the method preferably maintains the integrity of the sample such that the miRNA can be accurately detected and the amount measured according to the invention.

Methods for assessing an amount of nucleic acid expression in cells are well known in the art, and all suitable methods for assessing an amount of nucleic acid expression known to one of skill in the art are contemplated within the scope of the invention. The term “amount of nucleic acid expression” or “level of nucleic acid expression” as used herein refers to a measurable level of expression of the nucleic acids, such as, without limitation, the level of miRNA transcript expressed or a specific variant or other portion of the miRNA. The term “nucleic acid” includes DNA and RNA and can be either double stranded or single stranded. Non-limiting examples of suitable methods to assess an amount of nucleic acid expression may include arrays, such as microarrays, PCR, such as RT-PCR (including quantitative RT-PCR), nuclease protection assays and Northern blot analyses. In a specific embodiment, determining the amount of a miRNA comprises, in part, measuring the level of miRNA expression.

In one embodiment, the amount of nucleic acid expression may be determined by using an array, such as a microarray. Methods of using a nucleic acid microarray are well and widely known in the art. For example, a nucleic acid probe that is complementary or hybridizable to an expression product of a target gene may be used in the array. The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. The term “probe” as used herein refers to a nucleic acid sequence that will hybridize to a nucleic acid target sequence. In one example, the probe hybridizes to an RNA product of the nucleic acid or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In another embodiment, the amount of nucleic acid expression may be determined using PCR. A nucleic acid may be amplified using cDNA, mRNA or genomic DNA as a template and appropriate oligonucleotide primers according to standard PCR amplification techniques. Methods of PCR are well and widely known in the art, and may include quantitative PCR, semi-quantitative PCR, multiplex PCR, or any combination thereof. Specifically, the amount of nucleic expression may be determined using quantitative RT-PCR. Methods of performing quantitative RT-PCR are common in the art. In such an embodiment, the primers used for quantitative RT-PCR may comprise a forward and reverse primer for a target gene. The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less or more. The factors involved in determining the appropriate length of primer are readily known to one of ordinary skill in the art.

The amount of nucleic acid expression may be measured by measuring an entire miRNA transcript for a nucleic acid sequence, or measuring a portion of the miRNA transcript for a nucleic acid sequence. For instance, if a nucleic acid array is utilized to measure the amount of miRNA expression, the array may comprise a probe for a portion of the miRNA of the nucleic acid sequence of interest, or the array may comprise a probe for the full miRNA of the nucleic acid sequence of interest. Similarly, in a PCR reaction, the primers may be designed to amplify the entire cDNA sequence of the nucleic acid sequence of interest, or a portion of the cDNA sequence. One of skill in the art will recognize that there is more than one set of primers that may be used to amplify either the entire cDNA or a portion of the cDNA for a nucleic acid sequence of interest. Methods of designing primers are known in the art. Methods of extracting RNA from a biological sample are known in the art.

The level of expression may or may not be normalized to the level of a control nucleic acid. Such a control nucleic acid should not specifically hybridize with an miRNA nucleotide sequence of the invention. This allows comparisons between assays that are performed on different occasions. In certain embodiments, the level of expression is normalized to a control nucleic acid. In a specific embodiment, a control nucleic acid is selected from the group consisting of miR-191, miR-24 and miR-30c.

In certain embodiments, to classify the amount of miRNA as increased in a biological sample, the amount of miRNA in the biological sample compared to the reference value is increased at least 2-fold. For example, the amount of miRNA in the sample compared to the reference value is increased at least 2-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 200-fold, at least 300-fold, at least 400-fold, at least 500-fold, at least 1000-fold, at least 5000-fold, or at least 10000-fold. In a specific embodiment, the amount of miR-218 in the sample compared to the reference value is increased at least 10-fold. In another specific embodiment, the amount of miR-29b-3p in the sample compared to the reference value is increased at least 3-fold.

In certain embodiments, to classify the amount of miRNA as decreased in a biological sample, the amount of miRNA in the biological sample compared to the reference value is decreased at least 2-fold. For example, the amount of miRNA in the sample compared to the reference value is decreased at least 2-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 200-fold, at least 300-fold, at least 400-fold, at least 500-fold, at least 1000-fold, at least 5000-fold, or at least 10000-fold.

In another embodiment, the increase or decrease in the amount of miRNA is measured using p-value. For instance, when using p-value, a miRNA is identified as being differentially expressed between a a biological sample and a reference value when the p-value is less than 0.1, preferably less than 0.05, more preferably less than 0.01, even more preferably less than 0.005, the most preferably less than 0.001.

According to the disclosure, the subject may be treated if neurogenerative disease is detected, e.g., Huntington's disease. Additionally, the treatment modality may be altered if ineffectiveness of treatment or progression of motor neuron disease is detected. The term “treatment” or “therapy” as used herein means any treatment suitable for the treatment. Treatment may consist of standard treatments for MND. Non-limiting examples of standard treatment for MND include Riluzole (Rilutek), Tizanidine (Zanaflex), Baclofen, quinine, hyoscine hydrobromide skin patch, NSAIDs, gabapentin, physical therapy, acupuncture, immunotherapy, gene transfer therapy, stem cell and progenitor cell based cellular replacement therapy, antisense oligonucleotide therapy, antioxidant therapy, antidepressant therapy, antibody therapy, autophagy control therapy, drug therapy (small-molecule inhibitor of kynurenine 3- monooxygenase JM6), and any therapeutic agent known in the art or yet to be discovered. Still further, treatment may be as described below or with an agent as described in Section I.

Additional therapeutic agents may include those used in immunotherapy, gene transfer therapy, stem cell and progenitor cell based cellular replacement therapy, antisense oligonucleotide therapy, antioxidant therapy, antidepressant therapy, antibody therapy, autophagy control therapy, drug therapy (small-molecule inhibitor of kynurenine 3- monooxygenase JM6), and any therapeutic agent known in the art or yet to be discovered.

A miR-29b-3p modulating agent of the invention may be administered to a subject by several different means. For instance, compositions may generally be administered in dosage unit formulations containing conventional nontoxic pharmaceutically acceptable carriers, adjuvants, and vehicles as desired.

Methods of administration include any method known in the art or yet to be discovered. Exemplary administration methods include intravenous, intraocular, intratracheal, intratumoral, oral, rectal, topical, intramuscular, intraarterial, intrahepatic, intrathoracic, intrathecal, intracranial, intraperitoneal, intrapancreatic, intrapulmonary, or subcutaneously. A composition of the invention may also be administered directly by infusion into central nervous system fluid. One skilled in the art will appreciate that the route of administration and method of administration depend upon the intended use of the compositions, the location of the target area, and the condition being treated, in addition to other factors known in the art such as subject health, age, and physiological status.

In a preferred embodiment, the oligonucleotide may be administered parenterally. The term “parenteral” as used herein describes administration into the body via a route other than the mouth, especially via infusion, injection, or implantation, and includes intradermal, subcutaneous, transdermal implant, intracavernous, intravitreal, intra-articular or intrasynovial injection, transscleral, intracerebral, intrathecal, epidural, intravenous, intracardiac, intramuscular, intraosseous, intraperitoneal, intravenous, intrasternal injection, or nanocell injection. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In some embodiments, a miR-29b-3p modulating agent of the invention is administered parenterally. When miR-29b-3p modulating agent is administered parenterally, delivery methods are preferably those that are effective to circumvent the blood-brain barrier, and are effective to deliver agents to the central nervous system. For example, delivery methods may include the use of nanoparticles. The particles may be of any suitable structure, such as unilamellar or plurilamellar, so long as the antisense oligonucleotide is contained therein. Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known in the art. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc.

In another preferred embodiment, miR-29b-3p modulating agent is administered by continuous infusion into the central nervous system. Non-limiting examples of methods that may be used to deliver a miR-29b-3p modulating agent into the central nervous system by continuous infusion may include pumps, wafers, gels, foams and fibrin clots. In a preferred embodiment, miR-29b-3p modulating agent is delivered into the central nervous system by continuous infusion using an osmotic pump. An osmotic minipump contains a high-osmolality chamber that surrounds a flexible, yet impermeable, reservoir filled with the targeted delivery composition-containing vehicle. Subsequent to the subcutaneous implantation of this minipump, extracellular fluid enters through an outer semi-permeable membrane into the high-osmolality chamber, thereby compressing the reservoir to release the targeted delivery composition at a controlled, pre-determined rate. The targeted delivery composition, released from the pump, may be directed via a catheter to a stereotaxically placed cannula for infusion into the cerebroventricular space.

Compositions of the invention are typically administered to a subject in an amount sufficient to provide a benefit to the subject. This amount is defined as a “therapeutically effective amount.” A therapeutically effective amount may be determined by the efficacy or potency of the particular composition, the MND being treated, the duration or frequency of administration, the method of administration, and the size and condition of the subject, including that subject's particular treatment response. A therapeutically effective amount may be determined using methods known in the art, and may be determined experimentally, derived from therapeutically effective amounts determined in model animals such as the mouse, or a combination thereof. Additionally, the route of administration may be considered when determining the therapeutically effective amount. In determining the therapeutically effective amounts, one skilled in the art may also consider the existence, nature, and extent of any adverse effects that accompany the administration of a particular compound in a particular subject.

In some embodiments, when a miR-29b-3p modulating agent is administered by continuous infusion into the central nervous system, the miR-29b-3p modulating agent may be administered to the subject in an amount of about 0.1, 0.2, 0.3, 0.4, 0.5, 1, 1.5, 2, 2.5, 3, 3.5, 4, 4.5, 5, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or about 100 μg/day or more.

One of skill in the art will also recognize that the duration of the administration by continuous infusion can and will vary, and will depend in part on the subject, the neurodegenerative disease, and the severity, progression and improvement of the condition of the subject, and may be determined experimentally.

When a miR-29b-3p modulating agent is an antisense oligonucleotide, molecules of the invention are typically administered to a subject or generated in situ such that they hybridize with or bind to miR-29b-3p inhibiting the respective biological activity of miR-29b-3p. The hybridization may be by conventional nucleotide complementarity to form a stable duplex, or, for example, in the case of an antisense nucleic acid molecule which binds to DNA duplexes, through specific interactions in the major groove of the double helix. An antisense nucleic acid molecule of the invention may be administered by direct injection at a tissue site. Alternatively, antisense nucleic acid molecules may be modified to target selected cells and then administered systemically. For example, for systemic administration, antisense molecules may be modified such that they specifically bind to receptors or antigens expressed on a selected cell surface, e.g., by linking the antisense nucleic acid molecules to peptides or antibodies which bind to cell surface receptors or antigens. The antisense nucleic acid molecules may also be delivered by direct infusion into a subject. The antisense nucleic acid molecules may also be delivered to cells using gene therapy vectors known in the art. To achieve sufficient intracellular concentrations of the antisense molecules, vectors in which the antisense nucleic acid molecule is placed under the control of a strong pol II or pol III promoter are preferred.

As used herein, “subject” may refer to a living organism having a central nervous system. In particular, subjects may include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific value (e.g., captive or free specimens of endangered species), or mammals which otherwise have value. Suitable subjects may also include: mice, rats, dogs, cats, ungulates such as cattle, swine, sheep, horses, and goats, lagomorphs such as rabbits and hares, other rodents, and primates such as monkeys, chimps, and apes. In some preferred embodiments, a subject is a human. In other preferred embodiments, a subject is a rat. In yet other preferred embodiments, a subject is a mouse. Subjects may be of any age including newborn, adolescent, adult, middle age, or elderly.

A subject may be at risk for developing a neurodegerative disease resulting from dysregulation of miR-29b-3p. As such, in some embodiments, treating a neurodegerative disease resulting from dysregulation of miR-29b-3p prevents a disorder from developing in a subject at risk of developing or such that a disease or disorder is prevented, or delayed in its progression.

A subject may also be diagnosed as having a the neurodegerative disease resulting from dysregulation of miR-29b-3p

Treating a subject using a method of the invention may extend the survival of the subject. Alternatively, treating a subject using a method of the invention may extend the disease duration of the subject.

In some embodiments, treating a subject extends the survival of the subject. A method of the invention may extend the survival of a subject by days, weeks, months, or years, when compared to the survival of a subject that was not treated using a method of the invention. As will be recognized by individuals skilled in the art, the number of days, months, or years that a method of the invention may extend the survival of a subject can and will vary depending on the subject, the neurodegerative disease, and the condition of the subject when treatment was initiated among other factors.

In other embodiments, treating a subject extends the disease duration of a subject. As used herein, the term “disease duration” is used to describe the length of time between onset of symptoms and death caused by the disease. A method of the invention may extend the disease duration of a subject by days, weeks, months, or years, when compared to the survival of the subject that was not treated using a method of the invention. The number of days, months, or years that a method of the invention may extend the disease duration of a subject can and will vary depending on the subject, the neurodegerative disease, and the condition of the subject when treatment was initiated among other factors.

### II. METHODS

The present disclosure encompasses methods of treatment, and methods of predicting disease progression.

In some embodiments, the present disclosure encompasses methods of treatment of Huntington's disease in a subject in need thereof. Generally speaking, the methods comprise administering a composition detailed in section I above. In certain embodiments, a method of the present disclosure comprises administering a miR-29b-3p inhibitor to the subject, wherein administration results in enhanced neuronal autophagy. In particular embodiments, the miR-29b-3p inhibitor is an antisense RNA targeting miR-29b-3p. In some embodiments, a subject may also be administered glibenclamide, or a glibenclamide analog. For instance, the glibenclamide analog may be G2-115.

Methods of the present disclosure may also encompass administering glibenclamide, or a glibenclamide analog to a subject in need of treatment for Huntington's Disease, wherein administration results in enhanced neuronal autophagy. In particular embodiments, the glibenclamide analog is G2-115. As detailed in section I above, methods of making and administering glibenclamide or glibenclamide analogs are known in the art. In some embodiments, a subject may also be administered an miR-29b-3p inhibitor. For instance, the miR-29b-3p inhibitor may be an antisense RNA targeting miR-29b-3p.

In each of the above methods, compounds should be administered in pharmaceutically effective doses and routes, as detailed in section I above.

The present disclosure also encompasses methods of predicting Huntington's disease progression. Such methdos comprise obtaining a patient sample, determining the level of miR-29b-3p in the patient sample, comparing the level to a sample from (a) a healthy individual or (b) a previous sample from the patient to predict disease progression. That is to say, levels of miR-29b-3p have been found to correlate with disease progression, and identifying the level of miR-29b-3p in a sample may provide a prediction of where the subject is in disease progression.

### III. KITS

In still other aspects, the present invention provides articles of manufacture and kits containing materials useful for treating the conditions described herein. The article of manufacture may include a container of a composition as described herein with a label. Suitable containers include, for example, bottles, vials, and test tubes. The containers may be formed from a variety of materials such as glass or plastic. The container holds a composition having an active agent which is effective for treating, for example, conditions that benefit from activity and/or expression. The active agent is at least one miR-29b-3p modulating agent as disclsosed herein and may further include additional bioactive agents known in the art for treating the specific condition. The label on the container may indicate that the composition is useful for treating specific conditions and may also indicate directions for administration.

### Definitions

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein, “administering” is used in its broadest sense to mean contacting a subject with a composition of the invention.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A non-limiting example of stringent hybridization conditions are hybridization in 6× sodium chloride/sodium citrate (SSC) at about 45° C., followed by one or more washes in 0.2.x× SSC, 0.1% SDS at 50-65° C. (e.g., 50° C. or 60° C. or 65° C.). Preferably, the isolated nucleic acid molecule of the invention that hybridizes under stringent conditions corresponds to a naturally-occurring nucleic acid molecule. As used herein, a “naturally-occurring” nucleic acid molecule refers to a RNA or DNA molecule having a nucleotide sequence that occurs in a human cell in nature (e.g., encodes a natural protein).

As used herein, the term “nucleic acid molecule” is intended to include DNA molecules (e.g., cDNA or genomic DNA) and RNA molecules (e.g., mRNA or miRNA) and analogs of the DNA or RNA generated using nucleotide analogs. The nucleic acid molecule may be single-stranded or double-stranded.

An “isolated nucleic acid molecule” means that the material is removed from its original environment (e.g., the natural environment if it is naturally occurring). For example, a naturally occurring polynucleotide present in a living animal is not isolated, but the same polynucleotide or polypeptide, separated from some or all of the coexisting materials in the natural system, is isolated, even if subsequently reintroduced into the natural system. Such polynucleotides may be part of a vector or other composition and still be isolated in that such vector or composition is not part of its natural environment.

A “nucleic acid vector” is a nucleic acid sequence designed to be propagated and or transcribed upon exposure to a cellular environment, such as a cell lysate or a whole cell. A “gene therapy vector” refers to a nucleic acid vector that also carries functional aspects for transfection into whole cells, with the intent of increasing expression of one or more genes or proteins. In each case, such vectors usually contain a “vector propagation sequence” which is commonly an origin of replication recognized by the cell to permit the propagation of the vector inside the cell. A wide range of nucleic acid vectors and gene therapy vectors are familiar to those skilled in the art.

A miRNA is a small non-coding RNA molecule which functions in transcriptional and post-transcriptional regulation of gene expression. A miRNA functions via base-pairing with complementary sequences within mRNA molecules, usually resulting in gene silencing via translational repression or target degradation. A mature miRNA is processed through a series of steps from a larger primary RNA transcript (pri-miRNA), or from an intron comprising a miRNA (mirtron), to generate a stem loop pre-miRNA structure comprising the miRNA sequence. A pre-miRNA is then cleaved to generate the mature miRNA.

Primary miRNA transcripts are transcribed by RNA polymerase II and may range in size from hundreds to thousands of nucleotides in length (pri-mRNA). Pri-miRNAs may encode for a single miRNA but may also contain clusters of several miRNAs. The pri-miRNA is subsequently processed into an about 70 nucleotide hairpin (pre-miRNA) by the nuclear ribonuclease III (RNase III) endonuclease, Drosha. Thus, isolated nucleic acid molecules of the invention have various preferred lengths, depending on their intended targets. When targeted to pri-miRNA, preferred lengths vary between 100 and 200 nucleotides, e.g., 100, 120, 150, 180 or 200 nucleotides. In the cytoplasm, a second RNAse III, Dicer, together with its dsRBD protein partner, cuts the pre-miRNA in the stem region of the hairpin thereby liberating an about 21 nucleotide RNA-duplex. Thus, isolated polynucleotides of about 80, 70, 60, 50, 40, 30, 25, 21, 20, 19, 18, 17, 16, 15, 14, 13, 12, 11, 10, 9, 8, 7, or 6 nucleotides in length are also considered in one embodiment of the invention.

As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity are defined herein as sufficiently identical.

The term “sample” refers to a cell, a population of cells, biological samples, and subjects, such as mammalian subjects. The term “biological sample” refers to tissues, cells and biological fluids isolated from a subject, as well as tissues, cells and fluids present within a subject.

As used herein, “subject” refers to a living organism having a central nervous system. In particular, subjects may include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific value (e.g., captive or free specimens of endangered species), or mammals which otherwise have value. Suitable subjects may also include: mice, rats, dogs, cats, ungulates such as cattle, swine, sheep, horses, and goats, lagomorphs such as rabbits and hares, other rodents, and primates such as monkeys, chimps, and apes. In some embodiments, subjects may be diagnosed with a fibroblastic condition, may be at risk for a fibroblastic condition, or may be experiencing a fibroblastic condition. Subjects may be of any age including newborn, adolescent, adult, middle age, or elderly.

As used herein, the phrases “therapeutically effective amount” and “prophylactically effective amount” refer to an amount that provides a therapeutic benefit in the treatment, prevention, or management of pathological processes mediated by dysregulation of miR-29b-3p. The specific amount that is therapeutically effective may be readily determined by ordinary medical practitioners, and may vary depending on factors known in the art, such as the type of disorder being treated, the subject's history and age, the stage of the disorder, and administration of other agents in combination.

As used herein, a “pharmaceutical composition” includes a pharmacologically effective amount of a therapeutic agent of the invention and a pharmaceutically acceptable carrier. As used herein, “pharmacologically effective amount,” “therapeutically effective amount” or simply “effective amount” refers to that amount of an agent effective to produce the intended pharmacological, therapeutic or preventive result. For example, if a given clinical treatment is considered effective when there is at least a 15% reduction in a measurable parameter associated with a disease or disorder, a therapeutically effective amount of an agent for the treatment of that disorder or disease is the amount necessary to effect at least a 15% reduction in that parameter.

The term “pharmaceutically acceptable carrier” refers to a carrier for administration of a therapeutic agent. Such carriers may include, but are not limited to, saline, buffered saline, dextrose, water, glycerol, ethanol, and combinations thereof. The term specifically excludes cell culture medium. For drugs administered orally, pharmaceutically acceptable carriers may include, but are not limited to, pharmaceutically acceptable excipients such as inert diluents, disintegrating agents, binding agents, lubricating agents, sweetening agents, flavoring agents, coloring agents and preservatives. Suitable inert diluents may include sodium and calcium carbonate, sodium and calcium phosphate, and lactose, while corn starch and alginic acid are suitable disintegrating agents. Binding agents may include starch and gelatin, while the lubricating agent, if present, may generally be magnesium stearate, stearic acid or talc. If desired, the tablets may be coated with a material such as glyceryl monostearate or glyceryl distearate to delay absorption in the gastrointestinal tract.

As used herein, “percent complementarity” means the percentage of nucleotides of a modified oligonucleotide that are complementary to a microRNA. Percent complementarity may be calculated by dividing the number of nucleotides of the modified oligonucleotide that are complementary to nucleotides at corresponding positions in the microRNA by the total length of the modified oligonucleotide.

As used herein, “oligonucleotide” means a polymer of linked nucleosides, each of which may be modified or unmodified, independent from one another.

As used herein, “anti-miR” means an oligonucleotide having a nucleotides sequence complementary to a microRNA. In certain embodiments, an anti-m iR is a modified oligonucleotide.

As used herein, “internucleoside linkage” means a covalent linkage between adjacent nucleosides.

As used herein, “linked nucleosides” means nucleosides joined by a covalent linkage.

As used herein, “nucleobase” means a heterocyclic moiety capable of non-covalently pairing with another nucleobase.

As used herein, “nucleoside” means a nucleobase linked to a sugar.

As used herein, “nucleotide” means a nucleoside having a phosphate group or other internucleoside linkage forming group covalently linked to the sugar portion of a nucleoside.

As used herein, “modified oligonucleotide” means an oligonucleotide having one or more modifications relative to a naturally occurring terminus, sugar, nucleobase, and/or internucleoside linkage.

As used herein, “modified internucleoside linkage” means any change from a naturally occurring internucleoside linkage.

As used herein, “phosphorothioate internucleoside linkage” means a linkage between nucleosides where one of the non-bridging atoms is a sulfur atom.

As used herein, “modified sugar” means substitution and/or any change from a natural sugar.

As used herein, “modified nucleobase” means any substitution and/or change from a natural nucleobase.

As used herein, “5-methylcytosine” means a cytosine modified with a methyl group attached to the 5′ position.

As used herein, “2′fluoro sugar” means a sugar having a fluorine modification at the 2′ position.

As used herein, “2′-O-methyl sugar” or “2′-OMe sugar” means a sugar having an O-methyl modification at the 2′ position.

As used herein, “2′-O-methoxyethyl sugar” or “2′-MOE sugar” means a sugar having an O-methoxyethyl modification at the 2′ position.

As used herein, “2′-O-fluoro” or “2′-F” means a sugar having a fluoro modification at the 2′ position.

As used herein, “bicyclic sugar moiety” means a sugar modified by the bridging of two non-gem inal ring atoms.

As used herein, “locked nucleic acid (LNA) sugar moiety” means a substituted sugar moiety having a (CH₂)—O bridge between the 4′ and 2′ furanose ring atoms.

In practicing the present invention, many conventional techniques in molecular biology, microbiology, and recombinant DNA may be used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins eds.); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian cells, 1987 (J. H. Miller and M. P. Calos eds., Cold Spring Harbor Laboratory); and Methods in Enzymology, Vol. 154 and Vol. 155 (Wu and Grossman, and Wu, eds., respectively).

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Methods

### Plasmids, shRNA, and Cell Lines

The construction of all plasm ids used in this study are publicly available at Addgene as pTight-9-124-BclxL (#60857), rtTA-N144 (#66810), pmCTIP2-N106 (#66808), phMYT1L-N174 (#66809), phDLX1-N174 (#60859), phDLX2-N174 (#60860). Lentiviral human STAT3 shRNAs (TRCN0000329887) were obtained from Sigma. Lentiviral Vector human STAT3 cDNA (pLenti-GIII-EF1a, #456970610695) was obtained from Applied Biological Materials Inc. For visualize free autophagosomes (GFP and mCherry fluorescence) and autophagosomes, FUW mCherry-GFP-LC3 (#110060) was obtained from Addgene. For the overexpression of miR-29b-3p, the miRNA-29b-1 genomic sequence was cloned and ligated into the pLemir-turboRFP vector. For luciferase assay, full-length 3′UTR of STAT3 transcripts and 3′UTR mutagenizing miR-29b-3p target sites were cloned and ligated into pmirGLO vector. Adult dermal fibroblasts from symptomatic HD patients (Coriell NINDS and NIGMS Repositories: ND33947, ND30013, GM02173, GM04230, GM04198, GM02147), presymptomatic HD patients (GM04717, GM04861, GM04855, GM04831, GM04857, GM04829), and healthy controls (GM02171, AG11732, GM03440, GM00495, GM07492, GM08399, AG04453, AG10047, AG12956, GM02187, AG08379, AG11798) were acquired from the Coriell Institute for Medical Research.

### Lentivirus Preparation

Lentiviral production was carried out separately for each plasm id and transduced together as a single pooled cocktail. Briefly, the supernatant was collected 72 hours after the transfection (polyethyleneimine, Polysciences) of Lenti-X 293LE cells with each viral vector with the packaging plasm ids, psPAX2 and pMD2.G. Collected lentiviruses were filtered through 0.45 μm PES membranes and the Lenti-X concentrator (Clontech #631232) was added to concentrate the virus 4-fold. Lentivirus samples were spun at 1,500 g for 45 mins after overnight incubation and resuspended in 1/10 of the original volume with 1×PBS. Lentivirus and 7 ml of 20% sucrose cushion solution was added to centrifuge tubes and concentrated at 70,000 g for 2 hr at 4° C. Viral pellets were then resuspended in 10% sucrose solution and stored at −80° C. until transduction. The range of our typical lentivirus titer is 1×10⁷-2.5×10⁸ infection-forming units per milliliter (IFU/ml).

### MicroRNA-Mediated Neuronal Reprogramming

The lentiviral cocktail of rtTA, pTight-9-124-BclxL, CTIP2, MYT1L, DLX1, and DLX2 was added to human fibroblasts for 16 h, then cells were washed and fed with fibroblasts media containing 1 μg/mL doxycycline (DOX). Briefly, transduced fibroblasts were maintained in fibroblasts media containing DOX for two days before selection with Puromycin (3 mg/ml) on day 3, then plated onto poly-ornithine, fibronectin, and laminin-coated coverslips on day 5. Cells were subsequently maintained in Neurobasal A (Gibco) media containing B-27 plus supplement and GlutaMAX supplemented with valproic acid (1 mM), dibutyl cAMP (200 mM), BDNF (10 ng/ml), NT-3 (10 ng/ml), RA (1 mM), ascorbic acid (200 μM), and RVC (RevitaCell Supplement, 1×) until day 13. On day 14, BrainPhys (Stemcell) containing NeuroCult SM1 neuronal supplement and N2 supplement-A neurobasal media were added in half-to-half volume until analysis. DOX treatment was cycled every two days and half volume-feeding every 4 days. On day 6, Blasticidin (3 μg/μL) and G418 (300 μg/μL) were added to the media for selecting transcription factor-expressing cells. From day 10, media with supplements except Blasticidin and G418 were added in half-to-half volume. Ascorbic acid (200 μM), and RVC (RevitaCell Supplement, 1×) were stopped adding to media after day 21. Puromycin was added at a final concentration of 3 μg/μL and continued till further analyses.

### SYTOX Assay

0.1 μM SYTOX gene nucleic acid stain (Invitrogen, S7020) and 1 μl/mL of Hoechst 33342 (Thermo Scientific, 66249) were added into cell medium. Samples were incubated for at least 30 mins at 37° C. prior to live-cell imaging. Images were taken using Leica DMI 4000B inverted microscope with Leica Application Suite (LAS) Advanced Fluorescence.

### Apoptosis Assay in Live Cells

Reprogrammed cells grown in 96-well plates were treated with 1× Essen Bioscience IncuCyte® Caspase-3/7 Green Reagent (final concentration 5 μM) and 1× Essen Bioscience IncuCyte® Annexin V Green or Red Reagent on day 22 or 26. Image scheduling, collection, and analysis were conducted with the IncuCyte® S3 LiveCell Analysis System platform and IncuCyte S3 v2017A software. Treated plates were imaged every two hours for 7 days. At each timepoint, over 2 images were taken per well in brightfield, FITC, and TRITC channels. Images were analyzed for the number of green or red objects per well. For the apoptotic index, the number of green or red objects (i.n., fluorescence cells) divided by phase area (μm2) per well was quantified by the IncuCyte® S3 Live-Cell Analysis System.

### mCherry-GFP-LC3 Construct, Transduction, and Quantification

FUW mCherry-GFP-LC3 used was Addgene plasm id # 110060 ; http://n2t.net/addgene:110060; RRID:Addgene_110060. The concentrated lentivirus of mCherry-GFP-LC3 was added to reprogrammed MSNs at PID20. For imaging of cells expressing mCherry-GFP-LC3, cells were washed once with PBS, fixed and stained by TUBB3 antibody, after verification of expression of GFP and mCherry by microscopy at PID26. Images were captured using a Leica SP5X white light laser confocal system with Leica Application Suite (LAS) Advanced Fluorescence 2.7.3.9723. For quantification of autophagosome (i.e., mCherry+, GFP+ puncta) and autolysosome (i.e., mCherry+, GFP− puncta) compartments in MSNs from multiple HD patients and control individuals, measurements were performed in cells having at least 3 puncta per cell.

### Immunostaining Analysis

Reprogrammed cells were fixed with 4% paraformaldehyde (Electron Microscopy Sciences, #15710) for 20 min at room temperature (RT), and permeabilized with 0.2% Triton X-100 for 10 min at RT. Cells were blocked with 5% BSA and 1% goat serum in PBS and incubated with primary antibodies at 4° C. overnight, then incubated with secondary antibodies for 1 hr at RT. Cells were incubated with DAPI (Sigma, D-9542) for 5 minutes followed by washing with PBS. Images were captured using a Leica SP5X white light laser confocal system with Leica Application Suite (LAS) Advanced Fluorescence 2.7.3.9723.

### Immunoblot Analysis

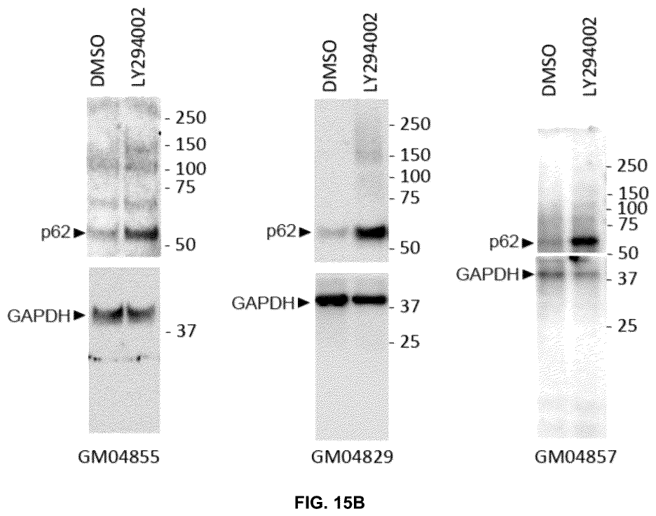

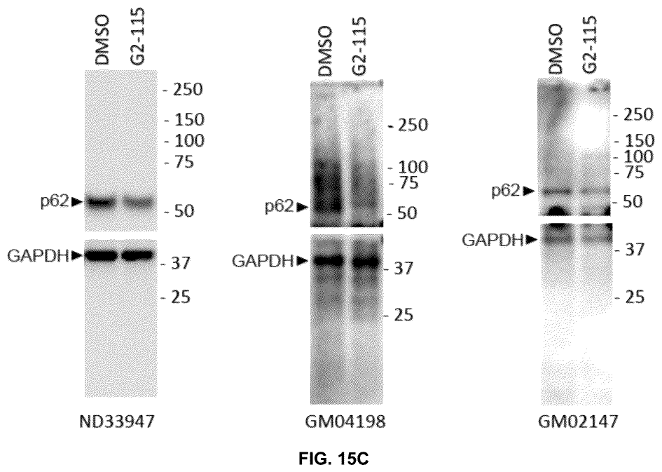

Cells were lysed in 2% SDS buffer. The concentrations of whole-cell lysates were measured using the Pierce BCA protein assay kit (Thermo Scientific, #23227). Equal amounts of whole-cell lysates were resolved by SDS-PAGE and transferred to a nitrocellulose membrane (GE Healthcare Life Sciences, #10600006) using a transfer apparatus according to the manufacturer's protocols (Bio-rad). After incubation with 5% BSA in TBS containing 0.1% Tween-20 (TBST) for 1 hour, membranes were incubated with primary antibodies at 4° C. overnight. Following the incubation with primary antibodies, membranes were incubated with a horseradish peroxidase-conjugated anti-mouse or anti-rabbit secondary antibodies for 30 min. Blots were developed with the ECL system (Thermo Scientific, #34080) according to the manufacturer's protocols. We provided unprocessed original images of immunoblots in FIG. 15.

### Antibodies

Primary antibodies used for immunostaining and immunoblot included rabbit anti-MAP2 (CST, #4542), mouse anti-tubulin β III (Covance, MMS-435P), rabbit anti-tubulin β III (Covance, PRB-435P-100), chicken anti-beta-tubulin 3 (Ayes Labs), rabbit anti-p62/SQSTM1 (Abcam, ab109012), mouse anti-p62/SQSTM1 (CST, #88588), rabbit anti-STAT3 (CST, #4904), rabbit anti-GAPDH (Santa Cruz, sc-32233), rabbit anti-DARPP-32(19A3) (CST, #2306), rat anti-CTIP2 (Abcam, ab18465), rabbit anti-DLX1 (Millipore, AB5724), rabbit anti-DLX2 (Abcam, ab135620), and rabbit anti-MYT1L (Proteintech, 25234-1-AP) antibodies. The secondary antibodies for immunostaining included goat anti-rabbit, mouse, rat or chicken IgG conjugated with Alexa-488, -568, -594, or -647 (Thermo Fisher Scientific).

### RNA Preparations and RT-qPCR

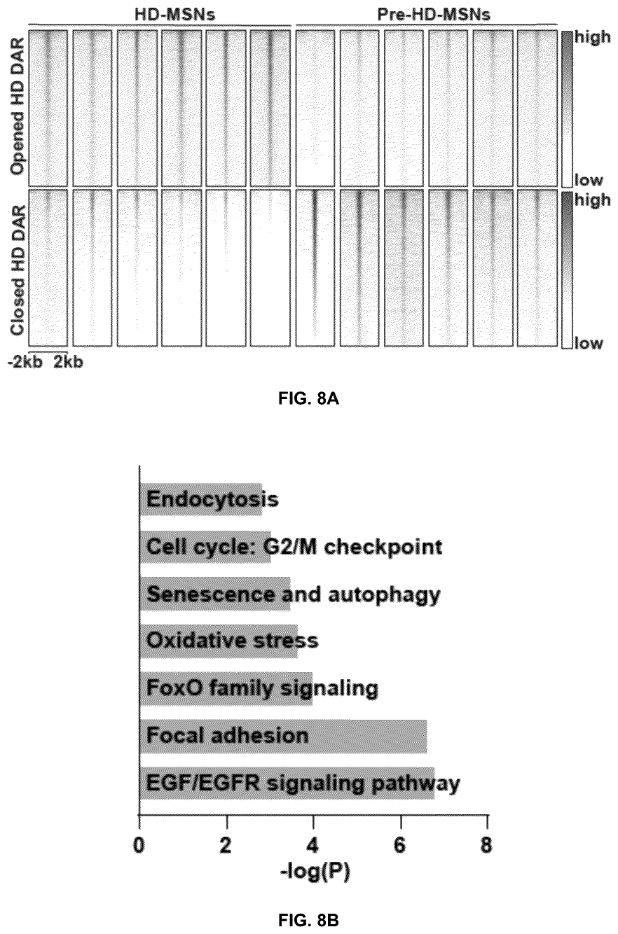

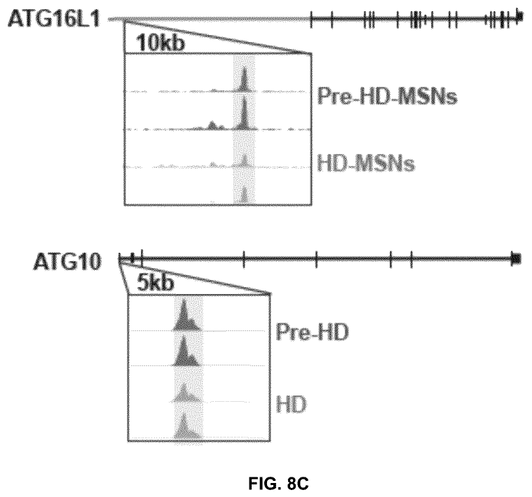

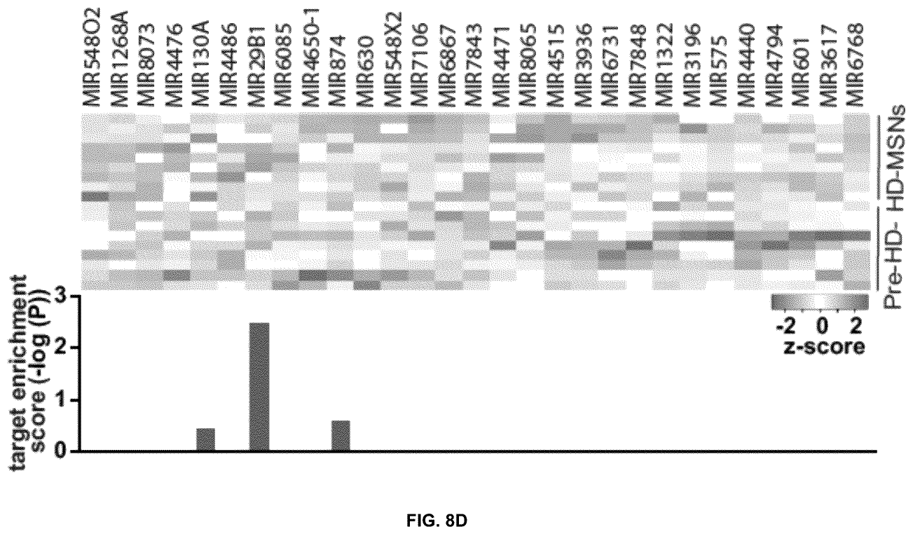

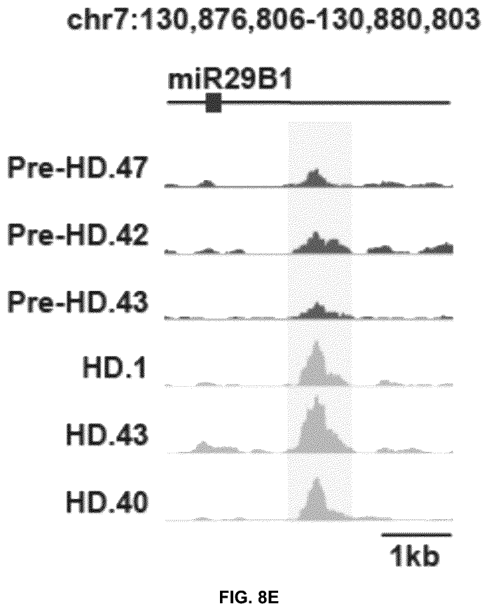

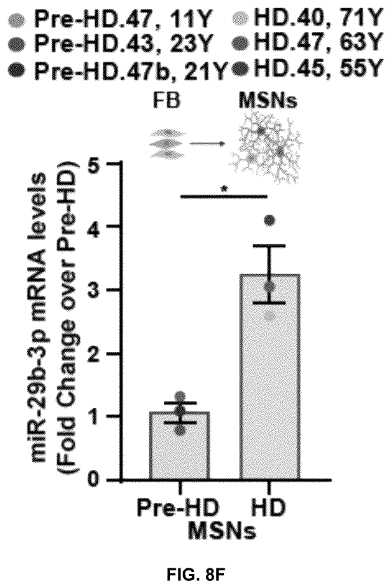

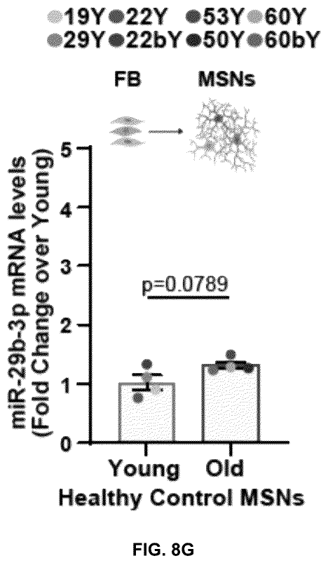

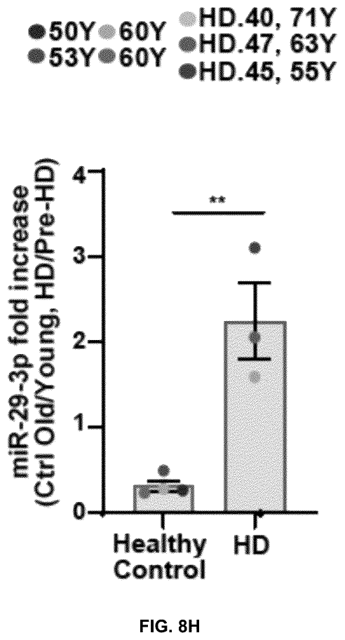

Total RNA from reprogramming cells was extracted using the RNeasy Micro Kit (Qiagen) or TRIzol Reagent (Invitrogen, 15596026). To verify miR-29b-3p levels, small RNA from the striatum of human brain samples (FIG. 8J) was extracted using TRIzol Reagent (Invitrogen, 15596026) and the RNA from the brain sections of healthy human control young and old samples was extracted using RNeasy FFPE Kit (Qiagen) (FIG. 8I). Reverse transcription was performed using the Superscript IV first strand synthesis system for RT-PCR (Invitrogen, 18090050) according to the manufacturer's protocol. Quantitative PCR was performed using SYBR Green PCR master mix (Applied Biosystems, 4309155) and StepOnePlus Real-Time PCR system (Applied Biosystems, 4376600) according to the manufacturer's protocol against target genes. Quantitative PCR analysis was done with the following primers: STAT3; 5′-CTTTGAGACCGAGGTGTATCACC-3′ (SEQ ID NO: 1) and 5′-GGTCAGCATGTTGTACCACAGG-3′ (SEQ ID NO: 2), DARPP-32; 5′-CCAAGGACCGCAAGAAGAT-3′ (SEQ ID NO: 3) and 5′-CTCCTCTGGTGAGGAGTGCT-3′ (SEQ ID NO: 4), GAPDH; 5′-ATGTTCGTCATGGGTGTGAA-3′ (SEQ ID NO: 5) and 5′-TGTGGTCATGAGTCCTTCCA-3′ (SEQ ID NO: 6). For the verification of mature miRNA-29b-3p, individual TaqMan probes for miR-29b-3p and miR-361-5p (as a housekeeping miRNA) were purchased (Life Technologies) and measurements by Taqman qPCR were carried out according to the manufacturer's protocol.

### Luciferase Assay

HEK 293 cells plated in a 96-well plate were transfected with 100 ng of pSilencer-miRNA, 100 ng of pmirGLO containing 3′UTR of interest, and PEI (Polysciences, 24765) with Opti-MEM (Life Technologies, 31985). Forty-eight hours after transfection, luciferase activity was assayed using Dual-Glo luciferase assay system (Promega, E2920) according to the manufacturer's protocol using Synergy H1 Hybrid plate reader (BioTek). Luciferase activity was obtained by normalizing firefly luminescence to renilla luminescence (luciferase activity=firefly/renilla) followed by normalizing to respective pSilencer-miR-NS control.

### RNA Sequencing

Samples were submitted to the Genome Access Technology Center (GTAC) at Washington University for library preparation and sequencing. Samples were prepared according to the library kit manufacturer's protocol, indexed, pooled, and sequenced on an Illumina HiSeq. Basecalls and demultiplexing were performed with Illumina's bcl2fastq software and a custom python demultiplexing program with a maximum of one mismatch in the indexing read. RNA-seq reads were then aligned to the Ensembl release 76 primary assemblies with STAR version 2.5.1a1. Gene counts were derived from the number of uniquely aligned unambiguous reads by Subread:feature Count version 1.4.6-p52. Isoform expression of known Ensembl transcripts was estimated with Salmon version 0.8.23. Sequencing performance was assessed for the total number of aligned reads, the total number of uniquely aligned reads, and features detected. The ribosomal fraction, known junction saturation, and read distribution over known gene models were quantified with RSeQC version 2.6.24. Pathway enrichment analysis (BioPlanet_2019 and Ontologies) was performed by Enrichr. The detailed information on pathway enrichment analysis is provided in Table 1.

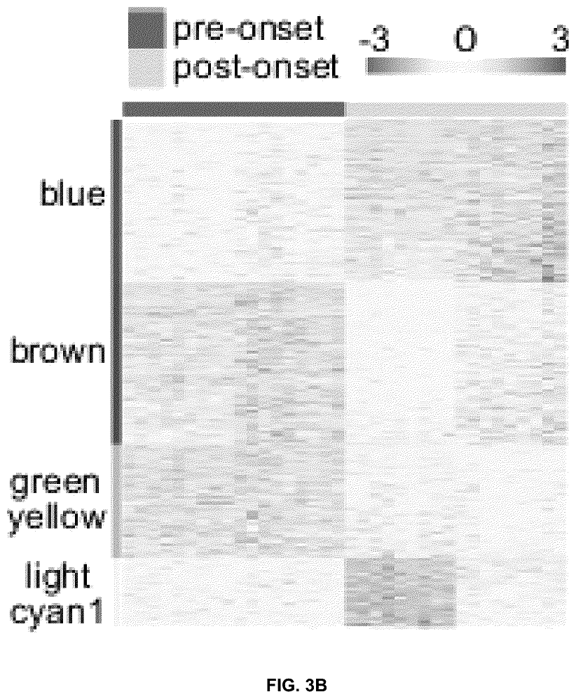

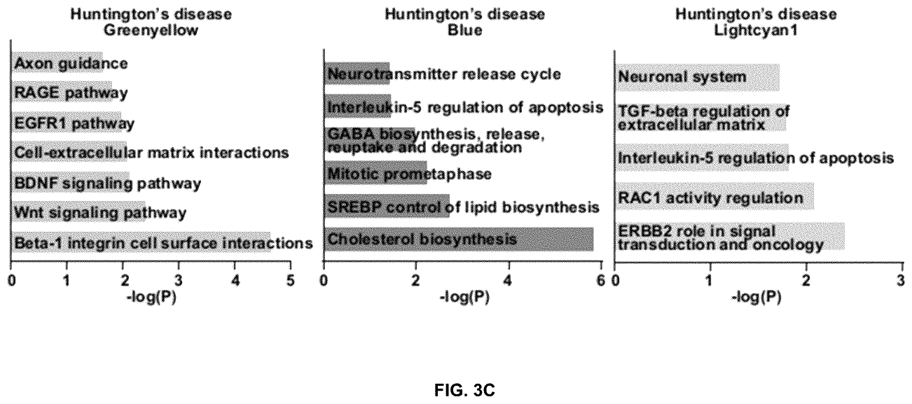

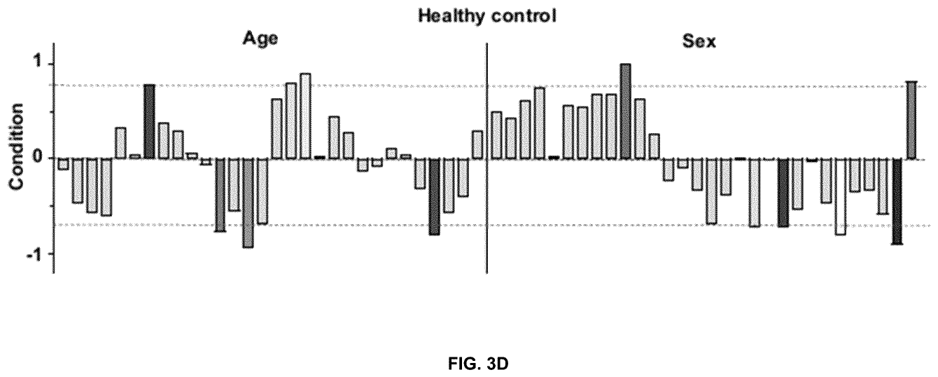

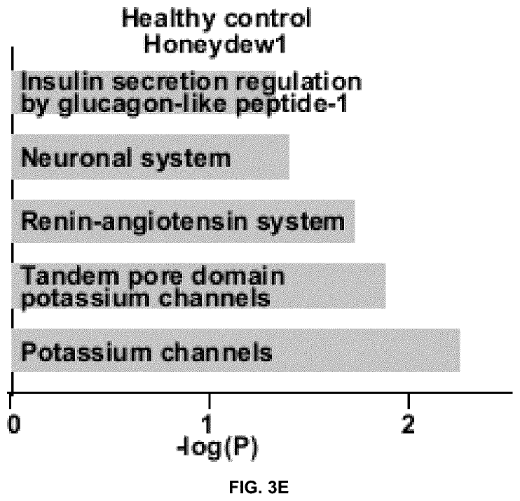

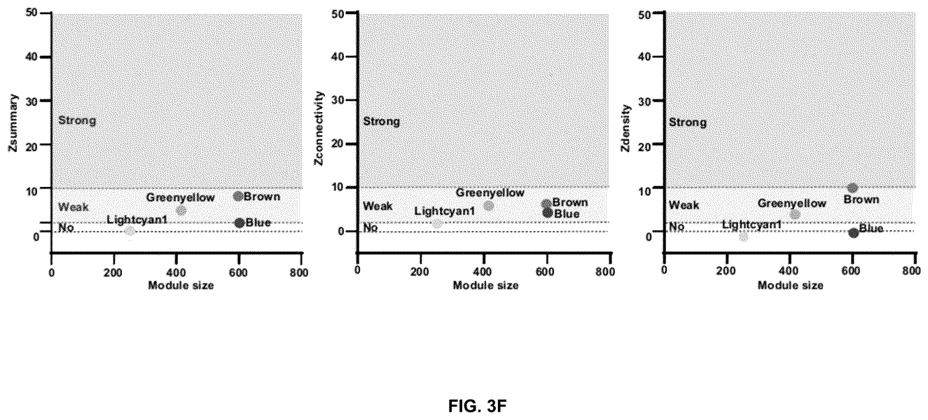

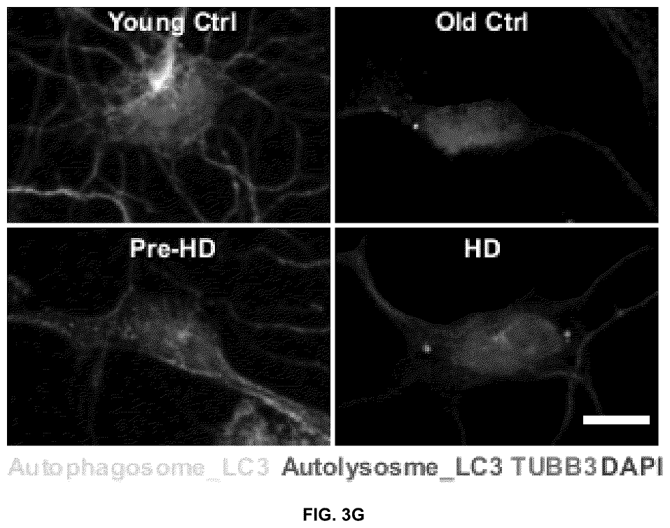

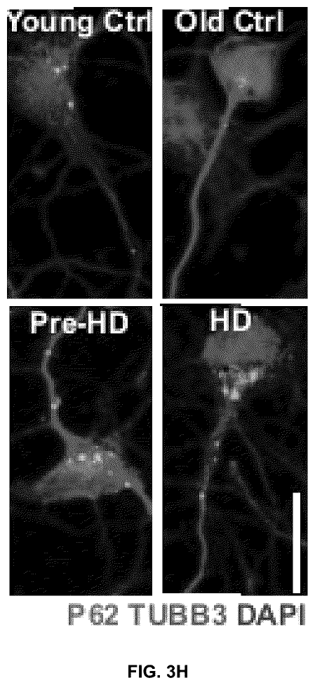

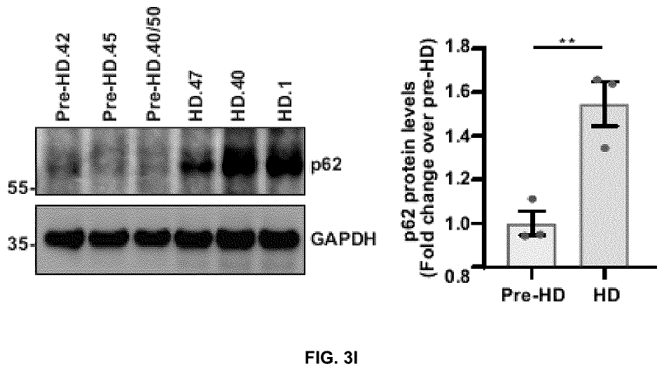

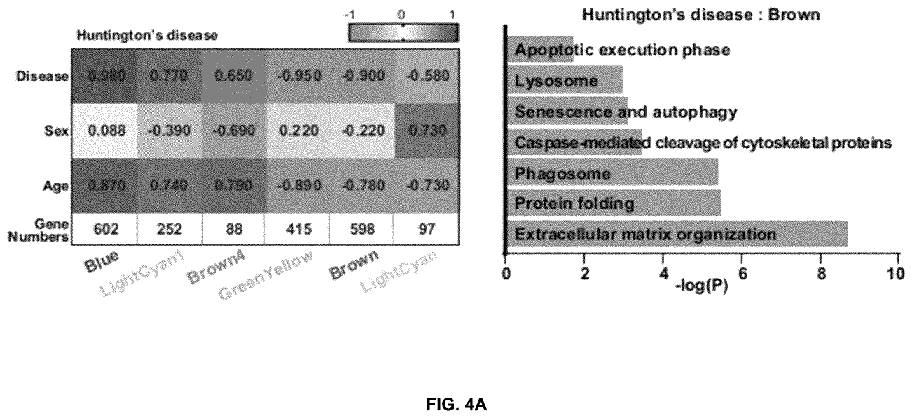

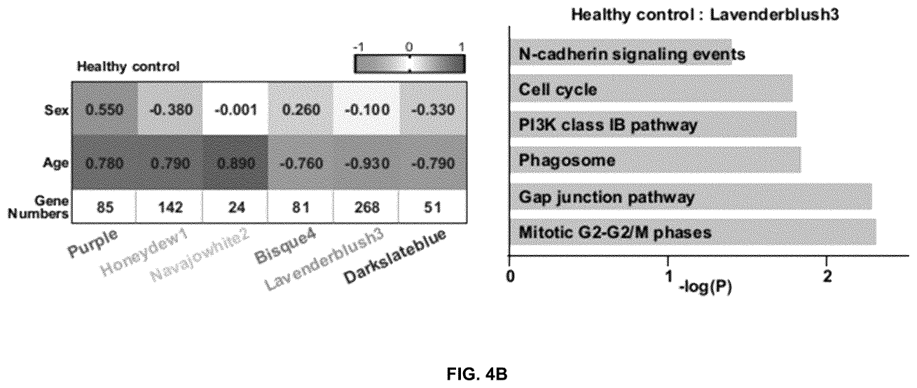

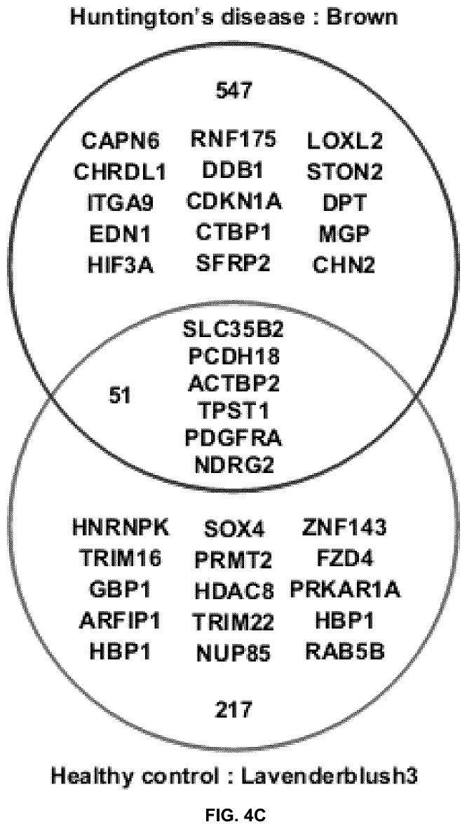

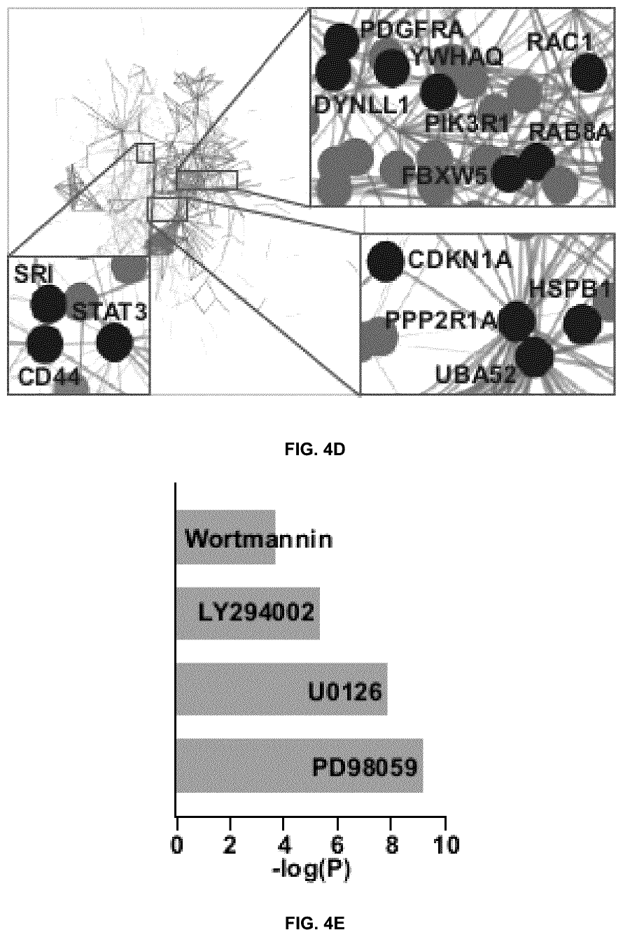

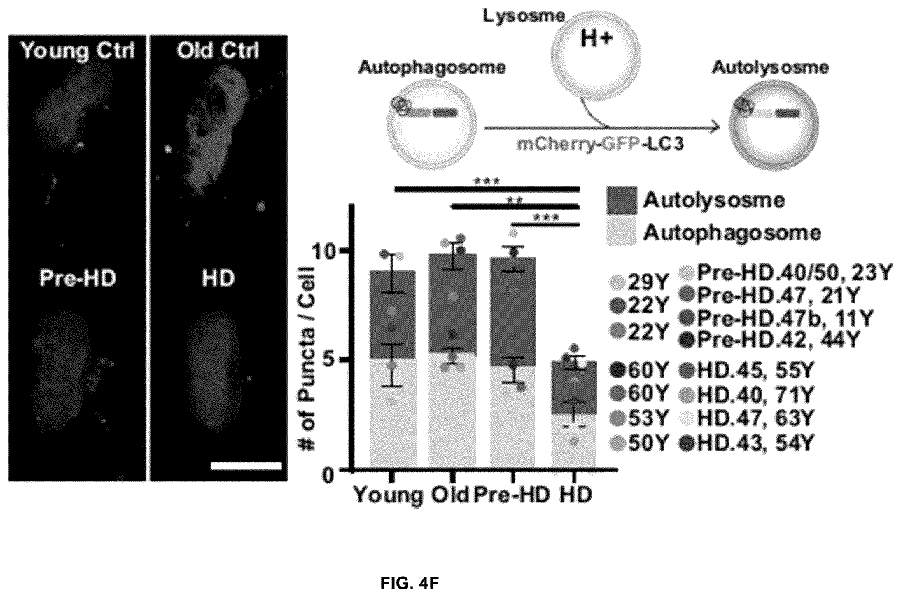

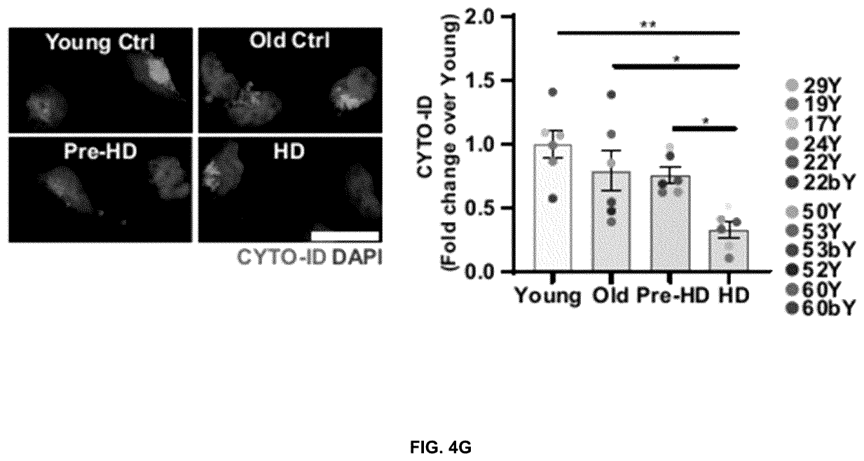

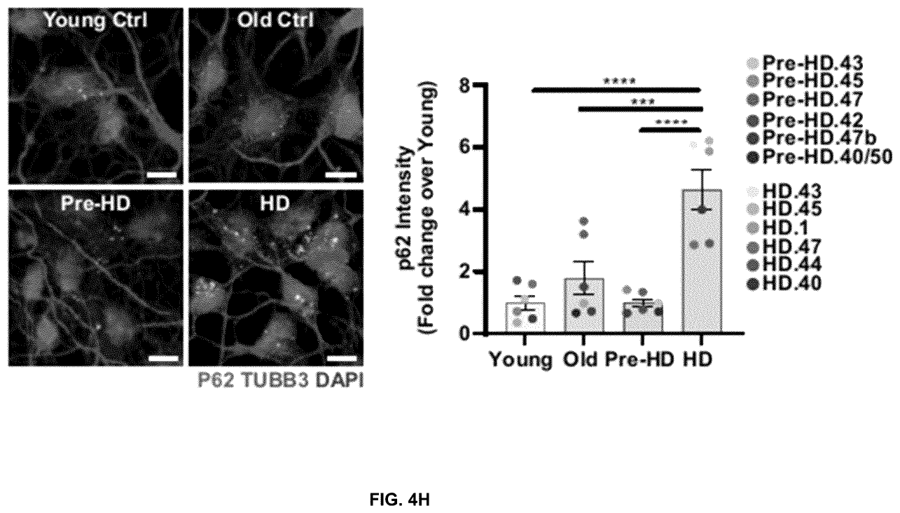

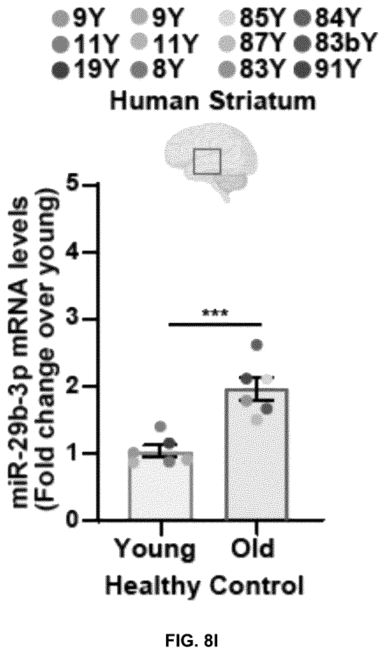

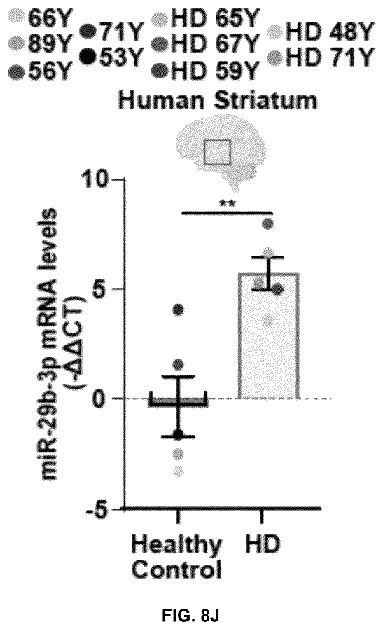

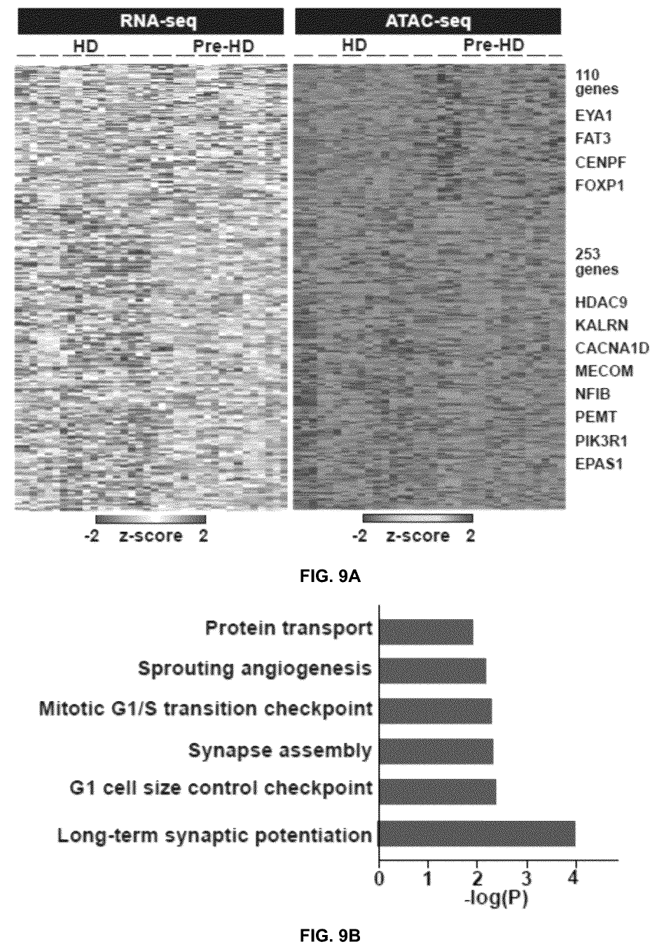

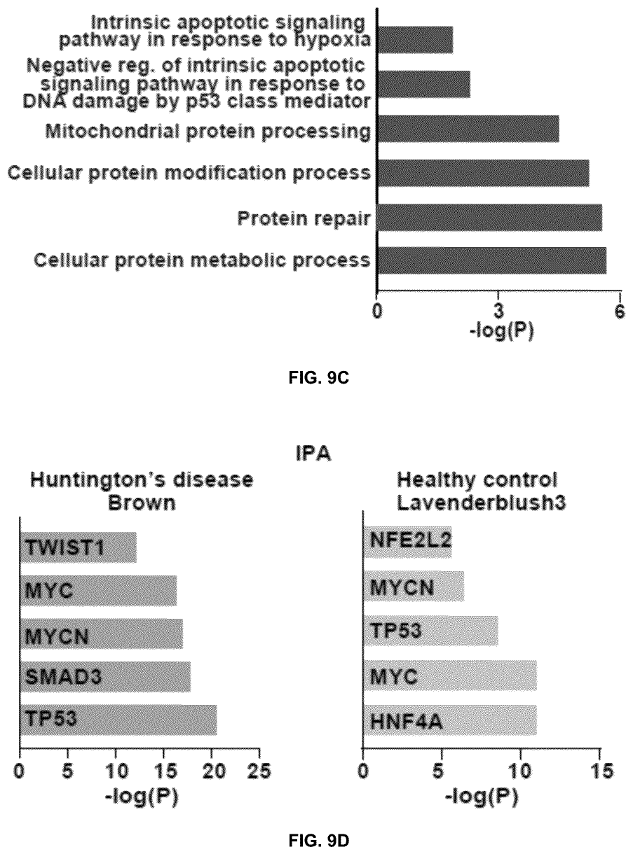

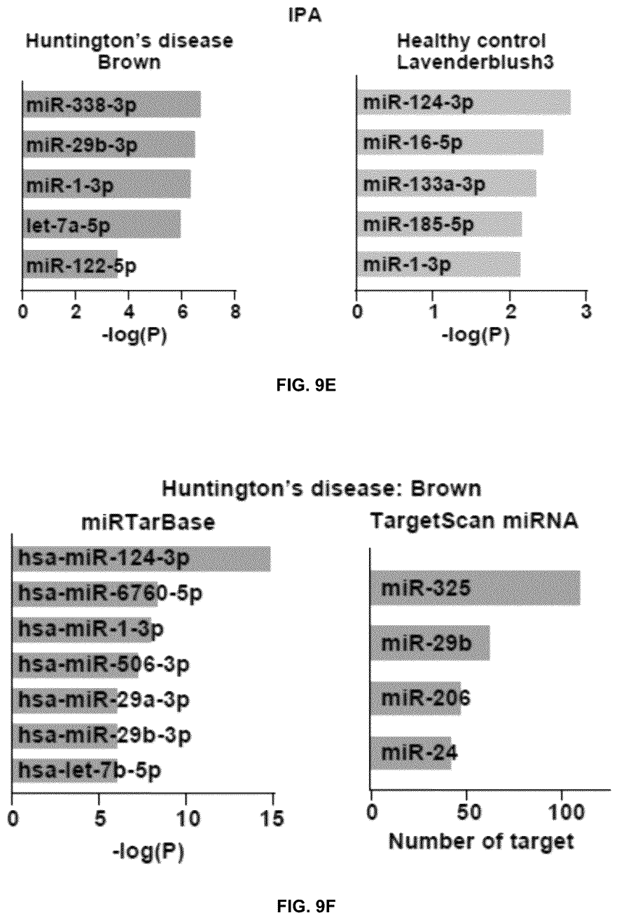

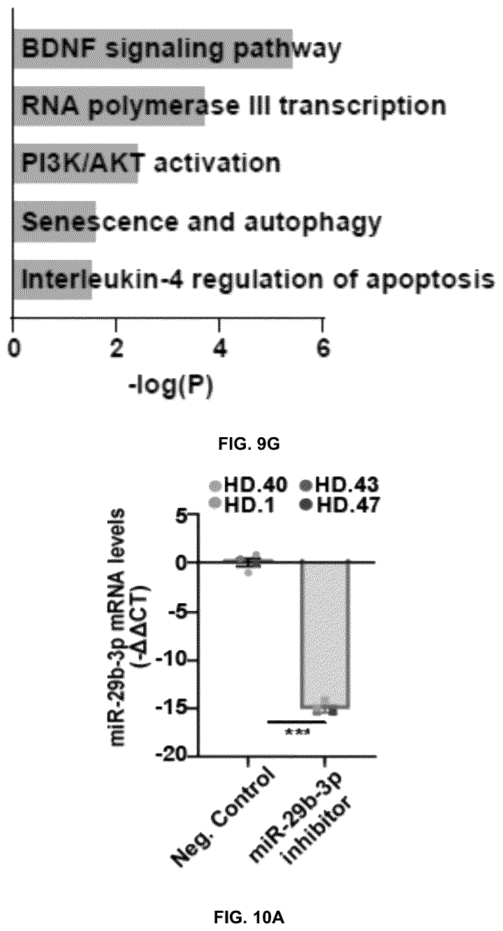

TABLE 1
Detailed information on pathway enrichment analysis

| | | | Adjusted | | Combined | |
|---|---|---|---|---|---|---|
| Figure | Term | P-value | P-value | Odds Ratio | Score | Genes |
| FIG. 4A | Extracellular | 2.04E−09 | 4.90E−07 | 7.44048374 | 148.8978962 | CRTAP; |
| | matrix | | | | | COL14A1; |
| | organization | | | | | MMP2; |
| | | | | | | PCOLCE; |
| | | | | | | PLOD1; |
| | | | | | | COL1A1; |
| | | | | | | MMP11; |
| | | | | | | MMP14; |
| | | | | | | COL3A1; |
| | | | | | | COL1A2; |
| | | | | | | COL5A1; |
| | | | | | | COL6A2; |
| | | | | | | COL5A2; |
| | | | | | | SERPINH1; |
| | | | | | | COL6A3; |
| | | | | | | TIMP1; |
| | | | | | | PPIB |
| | Protein folding | 3.24E−06 | 1.42E−04 | 7.65662079 | 96.77543501 | TUBA1C; |
| | | | | | | CCT2; |
| | | | | | | TUBA1B; |
| | | | | | | TUBB6; |
| | | | | | | FBXW5; |
| | | | | | | TUBB2A; |
| | | | | | | TBCD; |
| | | | | | | TUBB4B; |
| | | | | | | TUBB4A; |
| | | | | | | ACTB |
| | Phagosome | 3.78E−06 | 1.58E−04 | 4.11453949 | 51.3701733 | ITGB5; |
| | | | | | | TUBB; |
| | | | | | | HLAA; |
| | | | | | | TUBB4B; |
| | | | | | | TUBB4A; |
| | | | | | | ACTB; |
| | | | | | | MRC2; |
| | | | | | | SEC61A1; |
| | | | | | | TUBA1C; |
| | | | | | | TUBB6; |
| | | | | | | TUBA1B; |
| | | | | | | TUBB2A; |
| | | | | | | CANX; |
| | | | | | | CALR; |
| | | | | | | RAC1; |
| | | | | | | ITGA5; |
| | | | | | | ATP6V0D1 |
| | Caspase- | 3.23E−04 | 0.005909969 | 16.3249158 | 131.1961335 | GSN; |
| | mediated | | | | | CASP6; |
| | cleavage of | | | | | VIM; |
| | cytoskeletal | | | | | ADD1 |
| | proteins | | | | | |
| | Senescence | 7.58E−04 | 0.011091451 | 3.69047619 | 26.51550552 | COL1A1; |
| | and | | | | | BMP2; |
| | autophagy | | | | | CDKN1A; |
| | | | | | | MMP14; |
| | | | | | | SPARC; |
| | | | | | | GSN; |
| | | | | | | FN1; |
| | | | | | | MDM2; |
| | | | | | | SLC39A1; |
| | | | | | | CD44 |
| | Lysosome | 0.0010202 | 0.013822576 | 3.28654174 | 22.63699844 | CD63; |
| | | | | | | LAPTM4A; |
| | | | | | | NPC2; |
| | | | | | | GLB1; |
| | | | | | | FUCA1; |
| | | | | | | PSAP; |
| | | | | | | NAGA; |
| | | | | | | ACP2; |
| | | | | | | ATP6V0D1; |
| | | | | | | GUSB; |
| | | | | | | LGMN |
| | Apoptotic | 0.0192351 | 0.106345838 | 3.47224714 | 13.71891102 | GSN; |
| | execution | | | | | CASP6; |
| | phase | | | | | HIST1H1E; |
| | | | | | | VIM; |
| | | | | | | ADD1 |
| FIG. 4B | Mitotic G2- | 0.0048513 | 0.133013046 | 21.2737968 | 113.3574604 | TUBA1A; |
| | G2/M phases | | | | | TUBB |
| | Gap junction | 0.0051823 | 0.133013046 | 20.5454545 | 108.1204792 | TUBA1A; |
| | pathway | | | | | TUBB |
| | Phagosome | 0.0145496 | 0.141035924 | 11.8564593 | 50.15512059 | TUBA1A; |
| | | | | | | TUBB |
| | PI3K class IB | 0.0154927 | 0.141035924 | 72.3333333 | 301.4411523 | FZD4 |
| | pathway | | | | | |
| | Cell cycle | 0.0164108 | 0.141035924 | 6.19873016 | 25.47564696 | TUBA1A; |
| | | | | | | TUBB; |
| | | | | | | REC8 |
| | N-cadherin | 0.0400346 | 0.141035924 | 26.2753623 | 84.55442492 | GAP43 |
| | signaling | | | | | |
| | events | | | | | |
| FIG. 8B | EGF/EGFR | 1.76E−07 | 8.62E−05 | 2.4328545 | 37.83667246 | ERRFI1; |
| | signaling | | | | | INPPL1; |
| | pathway | | | | | ELK1; |
| | | | | | | PIK3C2B; |
| | | | | | | EPS8; |
| | | | | | | RPS6KA3; |
| | | | | | | RPS6KA2; |
| | | | | | | RPS6KA1; |
| | | | | | | STMN1; |
| | | | | | | AKT1; |
| | | | | | | JAK2; |
| | | | | | | AP2M1; |
| | | | | | | MAP3K4; |
| | | | | | | JAK1; |
| | | | | | | VAV3; |
| | | | | | | PIAS3; |
| | | | | | | USP8; |
| | | | | | | PRKCI; |
| | | | | | | RALBP1; |
| | | | | | | GAB1; |
| | | | | | | EPS15L1; |
| | | | | | | FOS; |
| | | | | | | GAB2; |
| | | | | | | DNM1; |
| | | | | | | VAV1; |
| | | | | | | VAV2; |
| | | | | | | DOK2; |
| | | | | | | PLSCR1; |
| | | | | | | CREB1; |
| | | | | | | RIN1; |
| | | | | | | SOS1; |
| | | | | | | CRK; |
| | | | | | | SOS2; |
| | | | | | | ARF6; |
| | | | | | | RALA; |
| | | | | | | PCNA; |
| | | | | | | RALB; |
| | | | | | | SHC1; |
| | | | | | | CAMK2A; |
| | | | | | | TWIST1; |
| | | | | | | PIK3R2; |
| | | | | | | PIK3R1; |
| | | | | | | PRKCZ; |
| | | | | | | FOXO1; |
| | | | | | | EGFR; |
| | | | | | | AURKA; |
| | | | | | | ATXN2; |
| | | | | | | ABL1; |
| | | | | | | AP2S1; |
| | | | | | | PTK2B; |
| | | | | | | CSK; |
| | | | | | | RICTOR; |
| | | | | | | PLCG1; |
| | | | | | | RALGDS; |
| | | | | | | MAPK4; |
| | | | | | | STAT5A; |
| | | | | | | JUN; |
| | | | | | | IQSEC1; |
| | | | | | | EGF; |
| | | | | | | STAT1; |
| | | | | | | TNK2; |
| | | | | | | PTK6; |
| | | | | | | PTPN12; |
| | | | | | | MAPK14; |
| | | | | | | PTK2; |
| | | | | | | SYNJ1; |
| | | | | | | SP1; |
| | | | | | | SPRY2; |
| | | | | | | KRAS; |
| | | | | | | BCAR1 |
| | Focal | 2.46E−07 | 9.02E−05 | 1.99305825 | 30.33107817 | ARAF; |
| | adhesion | | | | | TNC; |
| | | | | | | ELK1; |
| | | | | | | ACTB; |
| | | | | | | MYLK3; |
| | | | | | | MYLK; |
| | | | | | | IGF1R; |
| | | | | | | CCND3; |
| | | | | | | CCND1; |
| | | | | | | AKT2; |
| | | | | | | CAPN2; |
| | | | | | | AKT1; |
| | | | | | | MAP2K3; |
| | | | | | | PDGFRB; |
| | | | | | | PDGFRA; |
| | | | | | | ACTN1; |
| | | | | | | HGF; |
| | | | | | | ACTN4; |
| | | | | | | PGF; |
| | | | | | | COL4A1; |
| | | | | | | COL4A6; |
| | | | | | | MYL9; |
| | | | | | | VCL; |
| | | | | | | SHC4; |
| | | | | | | SHC1; |
| | | | | | | PXN; |
| | | | | | | PIK3R3; |
| | | | | | | PIK3R2; |
| | | | | | | PIK3R1; |
| | | | | | | MYL12A; |
| | | | | | | MYL12B; |
| | | | | | | PDGFC; |
| | | | | | | CHAD; |
| | | | | | | JUN; |
| | | | | | | TNK2; |
| | | | | | | TNK1; |
| | | | | | | FN1; |
| | | | | | | PARVA; |
| | | | | | | PTK6; |
| | | | | | | IGF1; |
| | | | | | | PARVB; |
| | | | | | | PTK2; |
| | | | | | | COL1A1; |
| | | | | | | DIAPH1; |
| | | | | | | COL1A2; |
| | | | | | | COL5A1; |
| | | | | | | COL5A3; |
| | | | | | | ITGA11; |
| | | | | | | BCL2; |
| | | | | | | ITGB1; |
| | | | | | | GSK3B; |
| | | | | | | ITGB5; |
| | | | | | | ITGB3; |
| | | | | | | ITGB2; |
| | | | | | | RASGRF1; |
| | | | | | | ILK; |
| | | | | | | PIK3CD; |
| | | | | | | ITGAE; |
| | | | | | | LAMC1; |
| | | | | | | ITGAX; |
| | | | | | | ITGAV; |
| | | | | | | VAV3; |
| | | | | | | PPP1R12A; |
| | | | | | | ITGA4; |
| | | | | | | ITGA3; |
| | | | | | | ITGA1; |
| | | | | | | VAV1; |
| | | | | | | VAV2; |
| | | | | | | COL6A1; |
| | | | | | | ITGA8; |
| | | | | | | COL6A3; |
| | | | | | | ITGA7; |
| | | | | | | ITGA6; |
| | | | | | | SOS1; |
| | | | | | | CRK; |
| | | | | | | DOCK1; |
| | | | | | | MET; |
| | | | | | | SOS2; |
| | | | | | | ITGA9; |
| | | | | | | LAMA5; |
| | | | | | | LAMA2; |
| | | | | | | ROCK1; |
| | | | | | | ROCK2; |
| | | | | | | SRC; |
| | | | | | | LAMA4; |
| | | | | | | LAMA3; |
| | | | | | | XIAP; |
| | | | | | | THBS1; |
| | | | | | | EGFR; |
| | | | | | | PAK1; |
| | | | | | | PAK6; |
| | | | | | | FLNB; |
| | | | | | | FYN; |
| | | | | | | FLNC; |
| | | | | | | MAPK4; |
| | | | | | | FARP2; |
| | | | | | | EGF; |
| | | | | | | LAMB2; |
| | | | | | | LAMB4; |
| | | | | | | VEGFC; |
| | | | | | | PPP1CA; |
| | | | | | | VEGFA; |
| | | | | | | MAPK10; |
| | | | | | | BCAR1 |
| | FoxO family | 1.14E−04 | 0.007752167 | 3.01628328 | 27.38162871 | RALA; |
| | signaling | | | | | RALB; |
| | | | | | | YWHAB; |
| | | | | | | ZFAND5; |
| | | | | | | FOXO3; |
| | | | | | | FOXO1; |
| | | | | | | CCNB1; |
| | | | | | | BCL2L11; |
| | | | | | | YWHAQ; |
| | | | | | | AKT1; |
| | | | | | | EP300; |
| | | | | | | YWHAG; |
| | | | | | | YWHAH; |
| | | | | | | CSNK1G3; |
| | | | | | | CREBBP; |
| | | | | | | GADD45A; |
| | | | | | | MST1; |
| | | | | | | CSNK1D; |
| | | | | | | CSNK1E; |
| | | | | | | SOD2; |
| | | | | | | SIRT1; |
| | | | | | | YWHAZ; |
| | | | | | | RBL2; |
| | | | | | | MAPK10; |
| | | | | | | BCL6; |
| | | | | | | SGK1; |
| | | | | | | CSNK1G2 |
| | Oxidative | 2.51E−04 | 0.012229551 | 4.17441176 | 34.6108906 | NQO1; |
| | stress | | | | | NFIX; |
| | | | | | | MAOA; |
| | | | | | | GSR; |
| | | | | | | CYBA; |
| | | | | | | FOS; |
| | | | | | | MAPK14; |
| | | | | | | SOD2; |
| | | | | | | NFKB1; |
| | | | | | | MAPK10; |
| | | | | | | GCLC; |
| | | | | | | SP1; |
| | | | | | | HMOX1; |
| | | | | | | NOX4; |
| | | | | | | JUNB; |
| | | | | | | XDH; |
| | | | | | | NFE2L2 |
| | Senescence | 3.60E−04 | 0.014680228 | 2.04986671 | 16.25419461 | GSK3B; |
| | and | | | | | CDKN1A; |
| | autophagy | | | | | CEBPB; |
| | | | | | | SPARC; |
| | | | | | | SRC; |
| | | | | | | PLAT; |
| | | | | | | CXCL1; |
| | | | | | | CXCL14; |
| | | | | | | ING1; |
| | | | | | | THBS1; |
| | | | | | | IGF1R; |
| | | | | | | IFI16; |
| | | | | | | IGFBP7; |
| | | | | | | ATG7; |
| | | | | | | SLC39A4; |
| | | | | | | ATG5; |
| | | | | | | MAP2K3; |
| | | | | | | SH3GLB1; |
| | | | | | | GABARAPL1; |
| | | | | | | JUN; |
| | | | | | | TGFB1; |
| | | | | | | GSN; |
| | | | | | | IGFBP5; |
| | | | | | | CDKN2A; |
| | | | | | | ATG10; |
| | | | | | | FN1; |
| | | | | | | HMGA1; |
| | | | | | | IGF1; |
| | | | | | | INHBA; |
| | | | | | | MAPK14; |
| | | | | | | MTOR; |
| | | | | | | CDC25B; |
| | | | | | | COL1A1; |
| | | | | | | IL6; |
| | | | | | | MMP14; |
| | | | | | | ATG16L1; |
| | | | | | | IRF1; |
| | | | | | | FKBP8; |
| | | | | | | BCL2; |
| | | | | | | IRF7; |
| | | | | | | PIK3C3; |
| | | | | | | IL6ST; |
| | | | | | | SQSTM1; |
| | | | | | | TP53; |
| | | | | | | CD44 |
| | Cell cycle: | 9.95E−04 | 0.02705043 | 3.57081977 | 24.68409343 | CDKN1A; |
| | G2/M | | | | | GADD45A; |
| | checkpoint | | | | | RASGRF1; |
| | | | | | | CDC25C; |
| | | | | | | CDC25A; |
| | | | | | | CDC25B; |
| | | | | | | CCNB1; |
| | | | | | | WEE1; |
| | | | | | | YWHAQ; |
| | | | | | | CHEK2; |
| | | | | | | RPS6KA1; |
| | | | | | | PI4KA; |
| | | | | | | EP300; |
| | | | | | | ATM; |
| | | | | | | TP53; |
| | | | | | | YWHAH |
| | Endocytosis | 0.0016669 | 0.038234499 | 1.56124856 | 9.986979424 | ZFYVE9; |
| | | | | | | CBLB; |
| | | | | | | IGF1R; |
| | | | | | | EEA1; |
| | | | | | | PIP5KL1; |
| | | | | | | PSD3; |
| | | | | | | VPS36; |
| | | | | | | GIT1; |
| | | | | | | AP2M1; |
| | | | | | | SH3GL1; |
| | | | | | | SH3GLB1; |
| | | | | | | PDGFRA; |
| | | | | | | SH3GLB2; |
| | | | | | | USP8; |
| | | | | | | PRKCI; |
| | | | | | | PDCD6IP; |
| | | | | | | HLA-B; |
| | | | | | | VPS37C; |
| | | | | | | HLA-C; |
| | | | | | | ARAP3; |
| | | | | | | HLA-A; |
| | | | | | | ARAP1; |
| | | | | | | ARFGAP3; |
| | | | | | | ARFGAP1; |
| | | | | | | DNM1; |
| | | | | | | TGFBR1; |
| | | | | | | HLA-E; |
| | | | | | | RNF41; |
| | | | | | | DNM2; |
| | | | | | | EPN2; |
| | | | | | | TGFBR2; |
| | | | | | | RUFY1; |
| | | | | | | DNM3; |
| | | | | | | EPN3; |
| | | | | | | ACAP2; |
| | | | | | | ZFYVE16; |
| | | | | | | RAB31; |
| | | | | | | DNAJC6; |
| | | | | | | CHMP4A; |
| | | | | | | MET; |
| | | | | | | ARF6; |
| | | | | | | CSF1R; |
| | | | | | | RAB5B; |
| | | | | | | RAB5C; |
| | | | | | | TSG101; |
| | | | | | | SRC; |
| | | | | | | VPS4A; |
| | | | | | | ASAP3; |
| | | | | | | AGAP1; |
| | | | | | | NEDD4L; |
| | | | | | | ASAP1; |
| | | | | | | CBL; |
| | | | | | | PRKCZ; |
| | | | | | | EGFR; |
| | | | | | | PLD2; |
| | | | | | | GRK5; |
| | | | | | | ERBB3; |
| | | | | | | GRK6; |
| | | | | | | AP2S1; |
| | | | | | | RAB11FIP5; |
| | | | | | | SMAD2; |
| | | | | | | SMAD3; |
| | | | | | | TGFB1; |
| | | | | | | IQSEC1; |
| | | | | | | EGF; |
| | | | | | | IQSEC3; |
| | | | | | | HSPA6; |
| | | | | | | SMAD6; |
| | | | | | | SMAD7; |
| | | | | | | EHD1; |
| | | | | | | EHD2; |
| | | | | | | EHD4; |
| | | | | | | NEDD4; |
| | | | | | | VPS45; |
| | | | | | | FGFR4; |
| | | | | | | FGFR3; |
| | | | | | | HSPA1B; |
| | | | | | | FGFR2 |
| FIG. 3C | Beta-1 integrin | 2.20E−05 | 0.007228959 | 11.9084337 | 127.7265022 | MDK; |
| Greenyellow | cell surface | | | | | COL6A1; |
| | interactions | | | | | TNC; |
| | | | | | | ITGA8; |
| | | | | | | CSPG4; |
| | | | | | | THBS1 |
| | Wnt signaling | 0.0039161 | 0.279387016 | 3.71287879 | 20.57925045 | GJA1; |
| | pathway | | | | | DAAM2; |
| | | | | | | SUFU; |
| | | | | | | F2R; |
| | | | | | | LRP5; |
| | | | | | | HDAC9; |
| | | | | | | DKK2 |
| | BDNF | 0.0075225 | 0.279387016 | 3.26933906 | 15.98661934 | MAST4; |
| | signaling | | | | | IGFBP3; |
| | pathway | | | | | SCHIP1; |
| | | | | | | CAMK4; |
| | | | | | | HMGB3; |
| | | | | | | PLAT; |
| | | | | | | CSPG4 |
| | Cell- | 0.0081519 | 0.279387016 | 16.6504202 | 80.08023366 | FBLIM1; |
| | extracellular | | | | | PARVA |
| | matrix | | | | | |
| | interactions | | | | | |
| | EGFR1 | 0.0101904 | 0.279387016 | 4.00851359 | 18.38427955 | MME; |
| | pathway | | | | | IGFBP3; |
| | | | | | | MT1F; |
| | | | | | | HMGB3; |
| | | | | | | PBXIP1 |
| | RAGE | 0.0149478 | 0.297550502 | 6.15727188 | 25.88019489 | GJA1; |
| | pathway | | | | | FABP4; |
| | | | | | | DDAH2 |
| | Axon | 0.0226102 | 0.297550502 | 2.60282066 | 9.863006898 | EFNB2; |
| | guidance | | | | | SEMA6A; |
| | | | | | | UNC5B; |
| | | | | | | SEMA3D; |
| | | | | | | COL6A1; |
| | | | | | | CRMP1; |
| | | | | | | EPHA2 |
| FIG. 3C Blue | Cholesterol | 1.56E−06 | 3.68E−04 | 31.5964912 | 422.4751625 | IDI1; |
| | biosynthesis | | | | | SQLE; |
| | | | | | | HMGCS1; |
| | | | | | | MSMO1; |
| | | | | | | HMGCR |
| | SREBP | 0.0019446 | 0.091786488 | 39.3333333 | 245.5455735 | HMGCS1; |
| | control of lipid | | | | | LDLR |
| | biosynthesis | | | | | |
| | Mitotic | 0.0058029 | 0.228247285 | 8.88772455 | 45.76643093 | CENPE; |
| | prometaphase | | | | | CENPF; |
| | | | | | | CLIP1 |
| | GABA | 0.0111698 | 0.376583102 | 13.8746499 | 62.36014365 | ALDH5A1; |
| | biosynthesis, | | | | | SYT1 |
| | release, | | | | | |
| | reuptake and | | | | | |
| | degradation | | | | | |
| | Interleukin-5 | 0.0346355 | 0.539034629 | 3.38898451 | 11.39673985 | IDI1; |
| | regulation of | | | | | GADD45A; |
| | apoptosis | | | | | IL21R; |
| | | | | | | HMGCR |
| | Neurotransmitter | 0.0374656 | 0.539034629 | 6.93137255 | 22.76493203 | ALDH5A1; |
| | release | | | | | SYT1 |
| | cycle | | | | | |
| | ERBB2 role in | 0.0039278 | 0.324388212 | 23.6952381 | 131.2639189 | ERBB4; |
| | signal | | | | | IL6R |
| | transduction | | | | | |
| | and oncology | | | | | |
| FIG. 3C | RAC1 activity | 0.008263 | 0.324388212 | 15.7873016 | 75.71529959 | TIAM1; |
| Lightcyan1 | regulation | | | | | ELMO1 |
| | Interleukin-5 | 0.0151833 | 0.324388212 | 6.10144928 | 25.55017631 | CLEC2B; |
| | regulation of | | | | | TFPI2; |
| | apoptosis | | | | | BIRC3 |
| | TGF-beta | 0.0160457 | 0.324388212 | 3.14994308 | 13.01654684 | PTGIS; |
| | regulation of | | | | | CLEC2B; |
| | extracellular | | | | | CREG1; |
| | matrix | | | | | TFPI2; |
| | | | | | | AOX1; |
| | | | | | | CRYAB |
| | Neuronal | 0.0190473 | 0.324388212 | 4.14273666 | 16.40867205 | CACNB2; |
| | system | | | | | GABRA3; |
| | | | | | | NEFL; |
| | | | | | | KCNA4 |
| FIG. 3E | Potassium | 0.0057347 | 0.16630518 | 19.5189003 | 100.7414672 | KCNB1; |
| | channels | | | | | KCNK1 |
| | Tandem pore | 0.0137167 | 0.187327456 | 82.5041322 | 353.8720301 | KCNK1 |
| | domain | | | | | |
| | potassium | | | | | |
| | channels | | | | | |
| | Renin- | 0.0193787 | 0.187327456 | 56.7073864 | 223.6301507 | ENPEP |
| | angiotensin | | | | | |
| | system | | | | | |
| | Neuronal | 0.041522 | 0.202037492 | 6.67547873 | 21.23824778 | KCNB1; |
| | system | | | | | KCNK1 |
| | Insulin | 0.0483236 | 0.202037492 | 21.5746753 | 65.36769141 | KCNB1 |
| | secretion | | | | | |
| | regulation by | | | | | |
| | glucagon-like | | | | | |
| | peptide-1 | | | | | |
| FIG. 9B | long-term | 1.24E−04 | 1.20E−01 | 37.1495327 | 334.0559818 | SYT1; |
| | synaptic | | | | | PTK2B; |
| | potentiation | | | | | PRKCZ |
| | (GO: 0060291) | | | | | |
| | G1 cell size | 4.82E−03 | 2.32E−01 | 21.6481481 | 115.4833407 | FHL1; |
| | control | | | | | E2F7 |
| | checkpoint | | | | | |
| | (GO: 0031568) | | | | | |
| | synapse | 4.94E−03 | 2.32E−01 | 4.05521092 | 21.53212389 | CSGALNACT1; |
| | assembly | | | | | CHRNA3; |
| | (GO: 0007416) | | | | | MYT1L; |
| | | | | | | PCDH10; |
| | | | | | | PCDHGB2; |
| | | | | | | APBA1 |
| | mitotic G1/S | 5.34E−03 | 2.45E−01 | 20.4444444 | 106.9812801 | FHL1; |
| | transition | | | | | E2F7 |
| | checkpoint | | | | | |
| | (GO: 0044819) | | | | | |
| | sprouting | 5.88E−03 | 2.63E−01 | 19.3674464 | 99.47654592 | PTK2B; |
| | angiogenesis | | | | | E2F7 |
| | (GO: 0002040) | | | | | |
| | protein | 0.0133521 | 0.285073508 | 12.2592593 | 52.91194008 | CENPF; |
| | transport | | | | | CEP290 |
| | (GO: 0015031) | | | | | |
| FIG. 9C | cellular protein | 5.15E−06 | 0.001242673 | 5.53687288 | 67.41876101 | IGFBP3; |
| | metabolic | | | | | PAPPA; |
| | process | | | | | MMP2; |
| | (GO: 0044267) | | | | | IGFBP2; |
| | | | | | | TNC; |
| | | | | | | MXRA8; |
| | | | | | | QSOX1; |
| | | | | | | FURIN; |
| | | | | | | APOE; |
| | | | | | | TGFBI; |
| | | | | | | SNCAIP; |
| | | | | | | SORL1 |
| | protein repair | 7.10E−06 | 0.001613126 | 5.3526971 | 63.45510097 | PAPPA; |
| | (GO: 0030091) | | | | | IGFBP3; |
| | | | | | | MMP2; |
| | | | | | | IGFBP2; |
| | | | | | | MXRA8; |
| | | | | | | TNC; |
| | | | | | | QSOX1; |
| | | | | | | FURIN; |
| | | | | | | APOE; |
| | | | | | | TGFBI; |
| | | | | | | SNCAIP; |
| | | | | | | SORL1 |
| | cellular protein | 9.86E−06 | 0.002003352 | 4.44230988 | 51.20613805 | IGFBP3; |
| | modification | | | | | MMP2; |
| | process | | | | | IGFBP2; |
| | (GO: 0006464) | | | | | TNC; |
| | | | | | | FURIN; |
| | | | | | | SNCAIP; |
| | | | | | | SORL1; |
| | | | | | | LOXL2; |
| | | | | | | PAPPA; |
| | | | | | | MXRA8; |
| | | | | | | QSOX1; |
| | | | | | | APOE; |
| | | | | | | TGFBI; |
| | | | | | | ST3GAL1 |
| | mitochondrial | 4.40E−05 | 0.007078062 | 4.39931657 | 44.13008924 | PAPPA; |
| | protein | | | | | IGFBP3; |
| | processing | | | | | MMP2; |
| | (GO: 0034982) | | | | | IGFBP2; |
| | | | | | | MXRA8; |
| | | | | | | TNC; |
| | | | | | | QSOX1; |
| | | | | | | FURIN; |
| | | | | | | APOE; |
| | | | | | | TGFBI; |
| | | | | | | SNCAIP; |
| | | | | | | SORL1 |
| | negative | 0.0041943 | 0.112209419 | 10.2907826 | 56.33200577 | MDM2; |
| | regulation of | | | | | SNAI2; |
| | intrinsic | | | | | CD44 |
| | apoptotic | | | | | |
| | signaling | | | | | |
| | pathway in | | | | | |
| | response to | | | | | |
| | DNA damage | | | | | |
| | by p53 class | | | | | |
| | mediator | | | | | |
| | (GO: 1902166) | | | | | |
| | intrinsic | 0.0150839 | 0.161284957 | 4.45183551 | 18.6715638 | CDKN1A; |
| | apoptotic | | | | | EPAS1; |
| | signaling | | | | | MDM2; |
| | pathway in | | | | | ZFP36L1 |
| | response to | | | | | |
| | hypoxia | | | | | |
| | (GO: 1990144) | | | | | |
| FIG. 9G | BDNF | 3.68E−06 | 0.001225343 | 12.6233291 | 157.9516351 | VCAN; |
| | signaling | | | | | IFITM2; |
| | pathway | | | | | NFIB; |
| | | | | | | DCX; |
| | | | | | | SERPINH1; |
| | | | | | | RND3; |
| | | | | | | TRIB2 |
| | RNA | 1.94E−04 | 0.016128856 | 30.2553191 | 258.6525249 | NFIX; |
| | polymerase III | | | | | NFIA; |
| | transcription | | | | | NFIB |
| | TPO signaling | 0.0017712 | 0.042129762 | 36.0996377 | 228.7304096 | STAT3; |
| | pathway | | | | | PIK3R1 |
| | PI3K/AKT | 0.0038576 | 0.048342987 | 23.7083333 | 131.7638483 | MDM2; |
| | activation | | | | | PIK3R1 |
| | Senescence | 0.0254701 | 0.111599145 | 8.52792096 | 31.29961077 | SPARC; |
| | and | | | | | MDM2 |
| | autophagy | | | | | |
| | Interleukin-4 | 0.0290943 | 0.118151182 | 4.75967118 | 16.83597324 | VCAN; |
| | regulation of | | | | | ID3; |
| | apoptosis | | | | | PTX3 |

### Omni-ATAC-Sequencing Preparation

Omni-ATAC was performed as outlined in Corces et al. (Corces et al., 2017). In brief, each sample was treated with DNase for 30 minutes prior to collection. Approximately 50,000 cells were collected for library preparation. Transposition reaction was completed with Nextera Tn5 Transposase (Illumina Tagment DNA Enzyme and Buffer Kit, Illumina) for 30 minutes at 37° C. and library fragments were amplified under optimal amplification conditions. Final libraries were purified by the DNA Clean & Concentrator 5 Kit (Zymo, USA). Libraries were sequenced on Illumina NovaSeq S4 XP (Genome Technology Access Center at Washington University in St. Louis).

### ATAC-Seq Analyses

ATAC-seq analysis in directly reprogrammed neurons was performed. Briefly, ATAC-seq reads were aligned to hg38 human genome assembly using bowtie2, and uniquely mapped reads were used for downstream analysis. Peaks for each sample were called using Homer findPeaks and combined altogether to make the own reference map for further differential analysis. Differential peaks were identified using DEseq2 with a cut-off of fold-change (FC) and adjusted p-value <0.05 and regarded as peaks gained or lost. Gained peaks in HD-MSNs were combined and defined as open (more accessible) chromatin regions. Conversely, all reduced peaks in HD-MSNs were defined as closed chromatin regions. Genes were annotated nearest to open or closed regions by using Homer and compared them with DEGs of RNA-seq data (adjusted p<0.05, log2FC≤−0.5 or log2FC≥0.5).

### Synthetic Route for the Preparation of G2-115

Preparation of intermediate 2: To a solution of 3,5-dichloroaniline (2 g, 12.34 mmol, 1 eq.) in DCM (30 mL) was added pyridine (3.42 g, 43.21 mmol, 3.5 eq.) and 3-ethoxyprop-2-enoyl chloride (1.99 g, 14.81 mmol, 1.2 eq.) at 0° C. The mixture was stirred at 15° C. for 2 hr. The reaction mixture was quenched by aqueous HCl (1M, 10 mL) and extracted with DCM (30 mL×3). The combined organic layers were washed with aq. sat. NaHCO3 (15 mL), dried over Na2SO4, filtered and concentrated to give (E)-N-(3,5-dichlorophenyl)-3-ethoxy-prop-2-enamide 2 (3.1 g, crude) as a yellow solid. ESI [M+H]=260.0.

Preparation of G2-115: A mixture of 2 (1.5 g, 5.77 mmol, 1 eq.) in sulfuric acid (15 mL, 98% purity) was stirred at 30° C. for 6 hr. The reaction mixture was poured into ice water (30 mL) and filtered. The filter cake was triturated with MeOH (8 mL×3) and the filter cake was dried to give 5,7-dichloro-1H-quinolin-2-one G2-115 (1.1 g, 4.97 mmol, 86.1% yield, 96.6% purity) as a brown solid. 1H NMR (400 MHz, DMSO-d6) δ=12.72-11.45 (m, 1H), 8.05 (br d, J=9.8 Hz, 1H), 7.48 (s, 1H), 7.33 (s, 1H), 6.71-6.59 (m, 1H). ESI [M+H]=214.0/216.0.

### Gene Co-Expression Network Construction by WGCNA

WGCNA was performed to construct gene co-expressed networks and identify co-expression gene modules. Expressed genes were normalized for sample depth (count per million read, CPM) and detected for outliers. The optimal soft threshold for adjacency computation was graphically determined, the transformed expression matrix was inputted into the WGCNA package functions, modules, and corresponding eigengenes were obtained. The dynamic clustering function was performed for gene hierarchical clustering dendrograms resulting in co-expression modules; correlation modules (r>0.7) were then merged. The dissimilarity of modules eigengenes (ME) was calculated, and the association between eigengenes values of clinical info was assessed by Pearson's correlation. For preservation statistics analysis, a total of 4 modules, blue, brown, greenyellow, and lightcyan1, were selected for module preservation calculation to determine which of the properties of the network module change across different experiments, pre-HD vs post-HD and healthy control old vs young groups. These modules contain most differentially expressed genes correlated between pre and post HD stages, also enough genes matched across datasets validate the preservation calculation. The datasets of HD and healthy control were used as reference and test set, respectively. The evidence of preservation was accessed by multiple statistical metrics.

### Sequencing Data Deposition

All the deep-sequencing data (RNA-seq and ATAC-seq) has been uploaded to the Gene Expression Omnibus (GEO) repository: GSE194243.

### Example 1: MSN Reprogramming of Patient Fibroblasts and Disease Stage-Dependent Manifestation of Neuronal Death

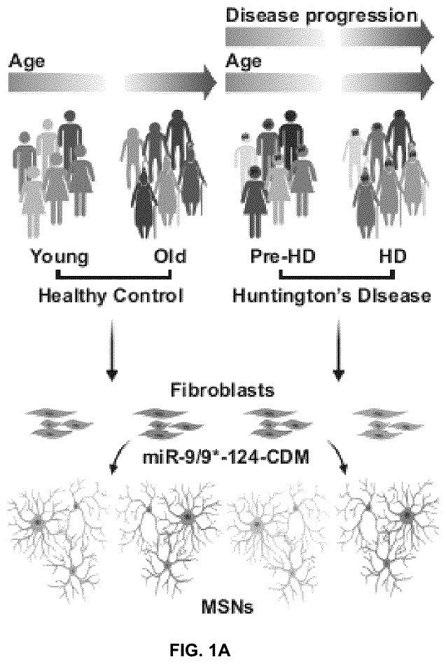

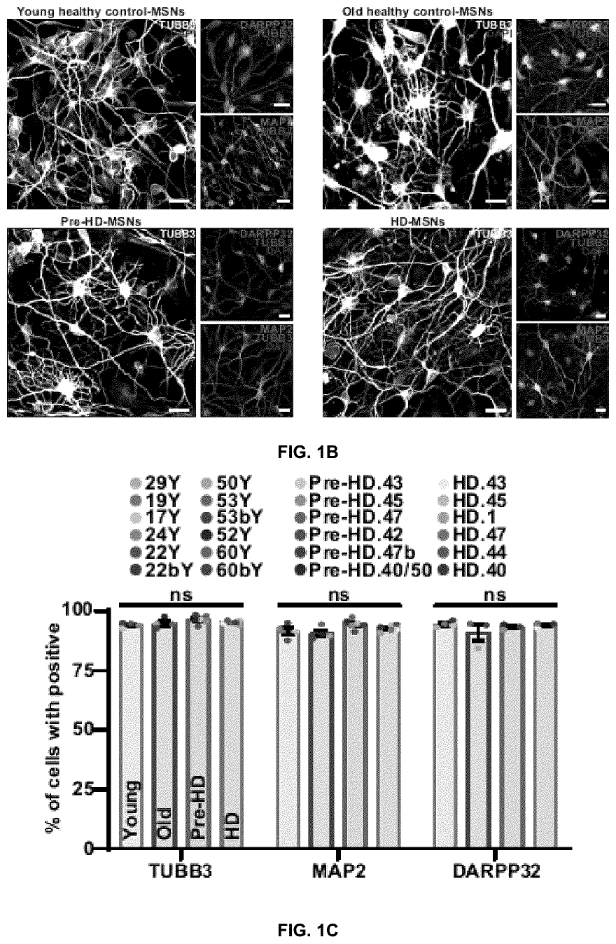

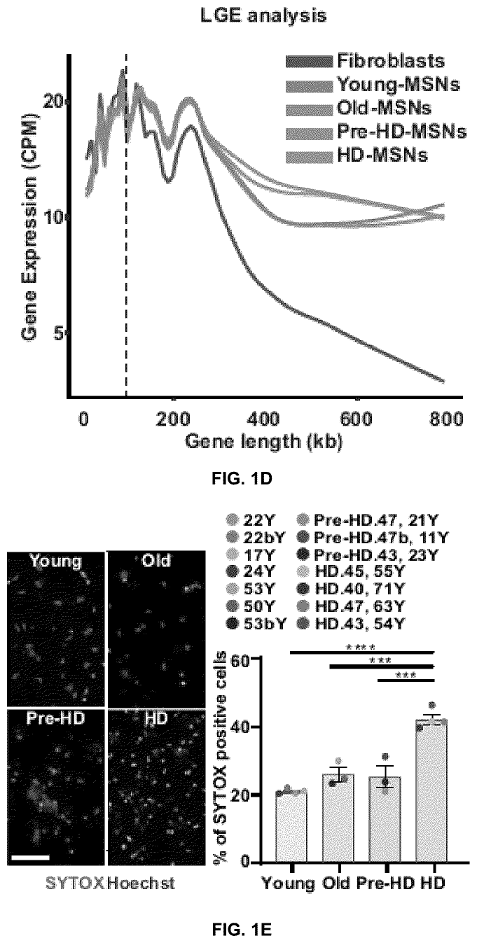

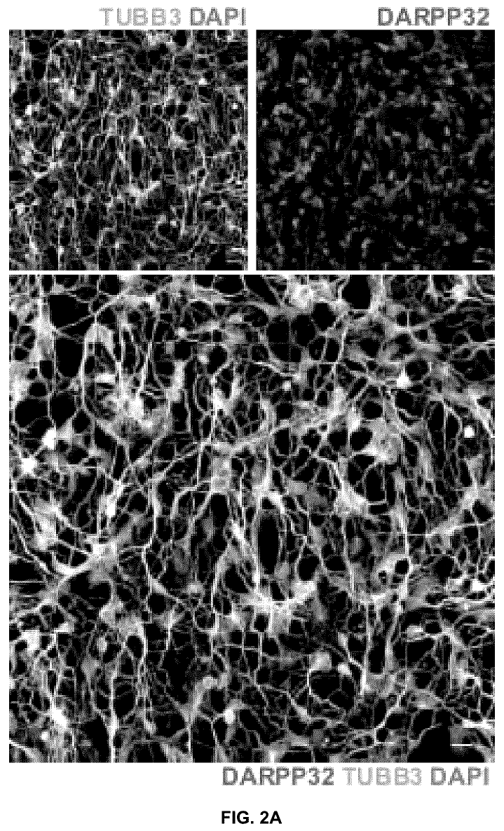

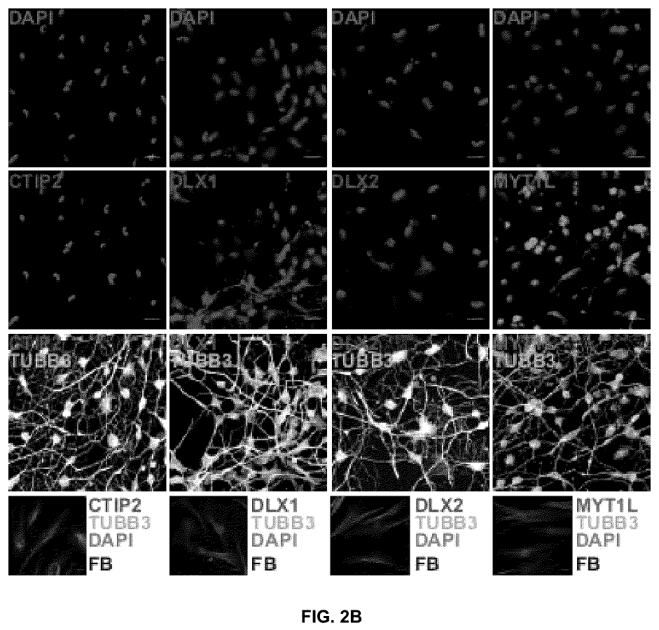

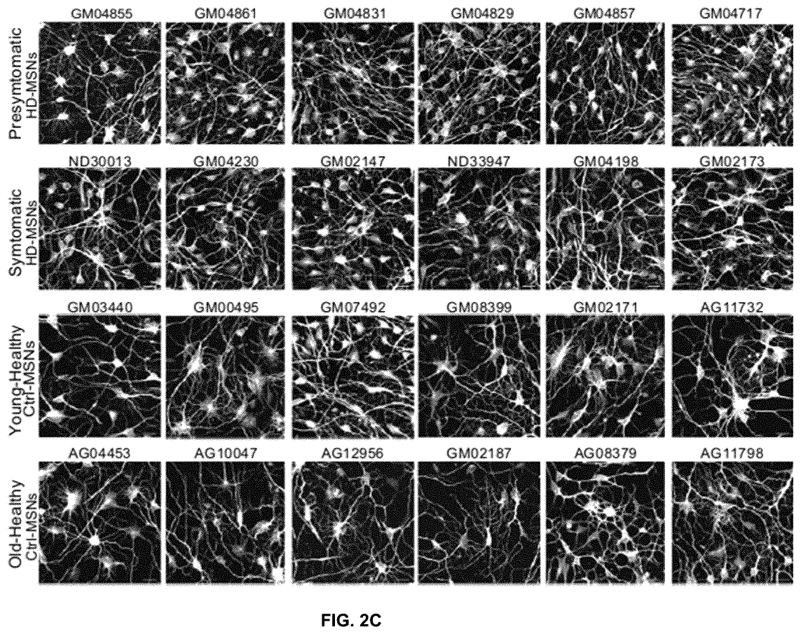

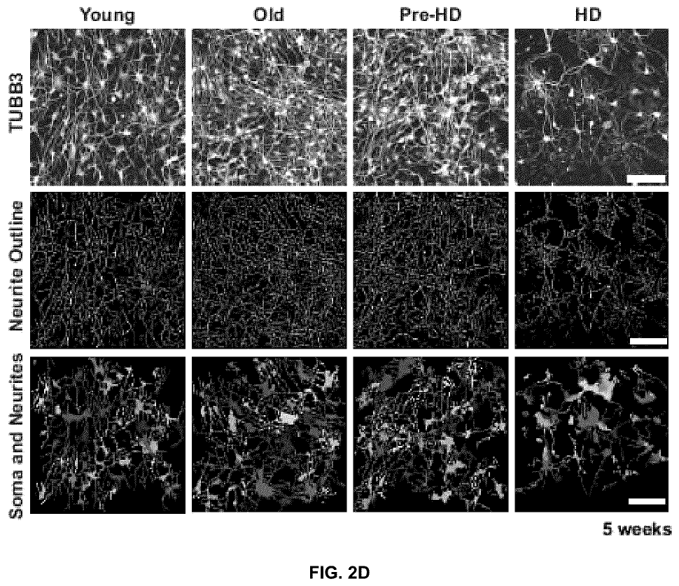

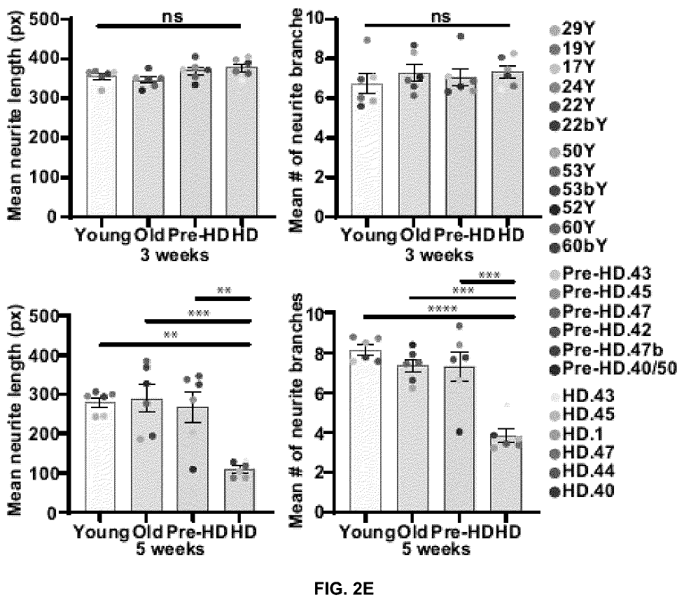

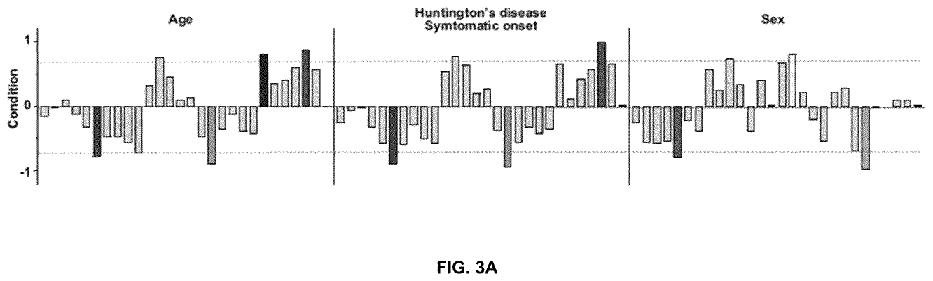

miR-9/9*-124-CDM-based MSN reprogramming was carried out in a total of 24 fibroblast samples, comprising six fibroblast lines from independent HD patients before clinical onset (11 to 44 years of age), six fibroblast lines from symptomatic patients (52 to 71 years of age), six control fibroblasts from healthy young adults (17-29 years of age), and six older control individuals (50 to 60 years of age) (FIG. 1A and Table 2). Pre-onset fibroblasts were collected 13-17 years before the reported age of the onset of HD symptoms, and all patient samples (pre- and post-onset) contained 40-50 CAG repeats (Table 2). 90% of the reprogrammed cells expressed neuronal markers MAP2, TUBB3, and an MSN marker, PPP1R1B (also known as DARPP-32), by post-induction day (PID) 21 or 30 across all samples (FIG. 1B-1C and FIG. 2A-2C), demonstrating consistent reprogramming efficiencies across fibroblast samples used in the study. To further confirm the neuronal identity of reprogrammed cells, long gene expression (LGE), a transcriptomic feature unique to functionally mature neurons, which provides an unbiased and stringent measure of neuronal identity in reprogrammed cells, was analyzed. LGE analysis by LONGO24 showed a drastic increase in LGE in converted cells over fibroblasts confirming successful neuronal reprogramming of fibroblast samples (FIG. 1D).

TABLE 2
The list of six pre-symptomatic HD patients, six HD patient
fibroblasts, and twelve healthy control fibroblasts

| | | | | | Age at | |
|---|---|---|---|---|---|---|
| | | | | Age at | Disease | |
| Line ID # | Genotype | Disease | Sex | Sampling | Onset | CAG |
| GM04855 | HD | Pre-Symptomatic | Male | 11 | 26 | 47 |
| GM04861 | HD | Pre-Symptomatic | Male | 16 | 33 | 45 |
| GM04831 | HD | Pre-Symptomatic | Male | 23 | 40 | 43 |
| GM04829 | HD | Pre-Symptomatic | Female | 21 | 34 | 47 |
| GM04857 | HD | Pre-Symptomatic | Female | 23 | 28 | 40/50 |
| GM04717 | HD | Pre-Symptomatic | Female | 44 | 60 | 42 |
| ND30013 | HD | Symptomatic | Male | 54 | 50 | 43 |
| GM04230 | HD | Symptomatic | Male | 55 | Not known | 45 |
| GM02147 | HD | Symptomatic | Male | 55 | Not known | 45 |
| ND33947 | HD | Symptomatic | Female | 71 | 63 | 40 |
| GM04198 | HD | Symptomatic | Female | 63 | Not known | 47 |
| GM02173 | HD | Symptomatic | Female | 52 | Not known | 44 |
| GM03440 | CTL | Healthy | Male | 22 | | |
| GM00495 | CTL | Healthy | Male | 29 | | |
| GM07492 | CTL | Healthy | Male | 17 | | |
| GM08399 | CTL | Healthy | Female | 19 | | |
| GM02171 | CTL | Healthy | Female | 22 | | |
| AG11732 | CTL | Healthy | Female | 24 | | |
| AG04453 | CTL | Healthy | Male | 52 | | |
| AG10047 | CTL | Healthy | Male | 53 | | |
| AG12956 | CTL | Healthy | Male | 50 | | |
| GM02187 | CTL | Healthy | Female | 60 | | |
| AG08379 | CTL | Healthy | Female | 60 | | |
| AG11798 | CTL | Healthy | Female | 53 | | |

Age is the primary factor that differs between pre- and post-onset samples. As directly reprogrammed neurons maintain the cellular age of starting fibroblast, it was explored whether neurodegeneration would be differentially manifested between young, old control-MSNs (Ctrl-MSNs), pre-HD-MSNs, and HD-MSNs. When the SYTOX-Green signal, a general cell death indicator, was measured, neuronal death was specifically increased only in HD-MSNs over pre-HD-MSNs and young and old Ctrl-MSNs (FIG. 1E). The morphological features such as average neurite lengths and the average number of neurite branches were also in these MSNs at both PID21 (3 weeks, early reprogramming phase) and PID35 (5 weeks, fully converted phase). The mean number of neurite length and the average number of neurite branches at PID21 did not differ between MSNs demonstrating consistent reprogramming efficiencies across all samples. However, at PID35, neurite length and branches were significantly decreased in HD-MSNs over healthy control- and pre-HD MSNs reflecting degenerating HD-MSNs (FIG. 2D). In summary, these results demonstrated that despite the mutant HTT present in both pre-HD-MSNs and HD-MSNs with similar repeat numbers, HD-MSNs' cell death phenotype correlated with the symptomatic stage of patients.

### Example 2: Comparative Gene Network Analysis Between Healthy Control, Pre- and Post-Onset Stages Identifies Age-Associated Alteration of Genetic Networks

To delineate cellular events underlying HD-MSN degeneration, the transcriptomes of pre-HD-MSNs (six independent patients), HD-MSNs (six independent patients), young-Ctrl-MSNs (six independent samples), and old-Ctrl-MSNs (six independent samples) were compared by RNA-seq (all with triple biological replicates, 72 samples total). RNA samples were collected after 21 days of reprogramming, a time point that aligns with the adoption of neuronal identity during miRNA-mediated reprogramming and prior to HD-MSN degeneration in culture. The weighted gene co-expression network analysis (WGCNA) was performed between pre-HD-MSNs and HD-MSNs samples to identify genes with expression changes correlated with donors' ages and disease stages. Gene modules with positive correlation values indicate increased expression of gene members in HD-MSNs whereas negative correlation values indicate decreased expression in HD-MSNs. Out of gene modules significantly related with stage, age, sex (FIG. 3A), six modules were most highly correlated (correlation value>0.7 or <−0.7) with age (p<10-7). Four of the six modules (brown, greenyellow, blue, and lightcyan1) also had a high correlation (correlation value >0.7 or <−0.7) with the disease stage (p<10-6) (FIG. 3A and FIG. 4A). Two (brown and greenyellow) of the four modules showed a correlation with downregulated genes in the symptomatic stage, HD-MSNs, whereas the other two modules (blue and lightcyan1) were correlated with upregulated genes (FIG. 4A and FIG. 3B).

Pathway enrichment analyses of the brown module (598 genes, age and post-symptomatic onset) revealed pathways enriched for cell death-related terms, such as apoptosis and caspase, protein folding, and senescence and autophagy (FIG. 4A), whereas the greenyellow module (415 genes) contained genes for neuronal function, such as the BDNF signaling pathway and axon guidance (FIG. 3C). The upregulated blue and lightcyan1 modules were enriched for other cellular functions, such as cholesterol biosynthesis, neurotransmitter release cycle, and neuronal system (FIG. 3C). Altogether, the brown module was found to represent the downregulated gene network that most closely resembled the degeneration phenotype in HD-MSNs compared to pre-HD-MSNs.

### Example 3: Comparative Gene Network Analysis Between Healthy Young and Older Ctrl-MSNs

Because the module-trait analysis of WGCNA in HD samples identified modules that were similarly affected by the age and disease progression (FIG. 3A and FIG. 4A), it was examined whether similar gene groups would appear between healthy Ctrl-MSNs from young and older age groups similarly matched to pre-HD-MSN and HD-MSN groups, respectively. Of the modules associated with age and sex conditions, six modules were found that were correlated with age (p<10-7) (FIG. 4B and FIG. 3D) in Ctrl-MSNs. Three modules showed a correlation with downregulated genes in old Ctrl-MSNs, whereas three modules showed the opposite correlation (FIG. 4B). Pathway enrichment analysis for the downregulated lavenderblush3 module (which had the highest number of gene members, 268) revealed an enrichment for terms not directly related to degeneration, including, for example, cell cycle-related pathways and cadherin signaling (FIG. 4B). The upregulated module, honeydewl was enriched for terms such as potassium channel and neuronal system (FIG. 3E). Next, the module preservation analysis was performed to test whether the gene modules detected in HD-MSNs would be manifested in healthy control gene expression data. Four modules (brown, blue, greenyellow, and lightcyan1) that were most highly correlated with age and disease stage in HD show respectively no or weak preservation across all healthy groups with Z summary statistic less than 8.0 (FIG. 3F). These results demonstrate that the HD modules reflect gene members more severely affected in pre- vs post-HD compared to young and old healthy control groups. Comparing the gene members from the age-/HD-associated brown module to the lavenderblush3 module from healthy controls showed neuronal death- and autophagy-related terms pronounced only in HD-MSNs. This is consistent with only 7% of the genes common between brown and lavenderblush3 modules (FIG. 4C), and age-associated gene network changes in HD-MSNs are distinct from the age module in Ctrl-MSNs (FIG. 4A-4B).

Because of the large number of genes and high correlation with both age and pathology onset in HD-MSNs, the brown module was examined to further dissect the relationship between gene members of the module. The coexpression dataset was integrated with a protein-protein interactions (PPI) network based on the experimental database of human protein-protein interactions (STRING interaction network) (FIG. 4D). One of the hubs in the brown module was signal transducers and activators of transcription 3 (STAT3) that regulate the balance between autophagy and cell death. Other hub proteins included RAB8A, RAC1, and PIK3R1 that play roles in regulating autophagy and cell death, as well as cell cycle regulators CDKN1A and UBA52 (FIG. 4D). Also, Upstream Regulator Analysis of the brown module by IPA predicted small molecule inhibitors of autophagy as upstream effectors, indicating that HD-MSNs behaved as if they had been treated with compounds that compromised autophagy (FIG. 4E).

### Example 4: Comparative Assessment of Autophagy Between Pre-HD-MSNs and HD-MSNs

Reflecting gene expression differences, it was examined whether pre-HD-MSNs and HD-MSNs would exhibit differential autophagy functions at a cellular level. First, tandem monomeric mCherry-GFP-tagged LC3, previously shown to distinguish prefusion autophagic compartments from mature acidic autolysosomes based on the differential pH sensitivity of GFP versus mCherry, was used. Notably, HD-MSNs from multiple symptomatic patients showed a reduction in the average number of pre-fusion autophagosomes (mCherry-positive; GFP-positive) and post-fusion autolysosomes (mCherry-positive; GFP-negative) per cell compared to pre-HD-MSNs and Ctrl-MSNs (both young and old) (FIG. 4F and FIG. 3G), suggesting an overall and specific decrease in autophagy in HD-MSNs. CYTO-ID assay, a fluorescence-based live-cell assay for accumulated autophagic vacuoles, and immunostaining of p62/SQSTM1, a marker widely used to monitor autophagic activity due to its binding to LC3 and degradation during autophagy, were further performed. HD-MSNs consistently showed lower CYTO-ID signals (FIG. 4G) and increased levels of p62/SQSTM1 compared to pre-HD-MSNs and young and old Ctrl-MSNs (FIG. 3H and FIG. 4H). The difference in p62 between pre-HD-MSNs and HD-MSNs was also validated by immunoblot assay (FIG. 3I). Because caspase activation has been detected in HD, and cell death-related pathways (apoptosis and caspase) were enriched in the brown module, the levels of caspase activation between pre-HD-MSN and HD-MSNs were compared. Live-cell monitoring of Caspase-3/7 Green Dye staining and Annexin V signal (an apoptotic marker via its ability to bind to phosphatidylserine on the extracellular surface) showed significantly higher levels of caspase activation by PID26 and apoptotic signal by PID30 (FIG. 4I). In summary, results indicated a significantly lower level of autophagy activity and increased cell death marks in HD-MSNs compared to pre-HD-MSNs and young and old Ctrl-MSNs.

### Example 5: Autophagy Inhibition Induces Degeneration of Pre-HD-MSNs

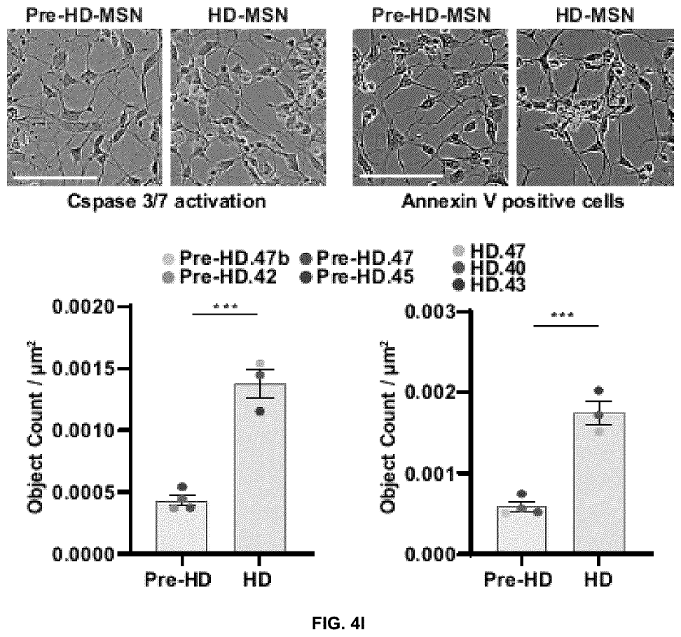

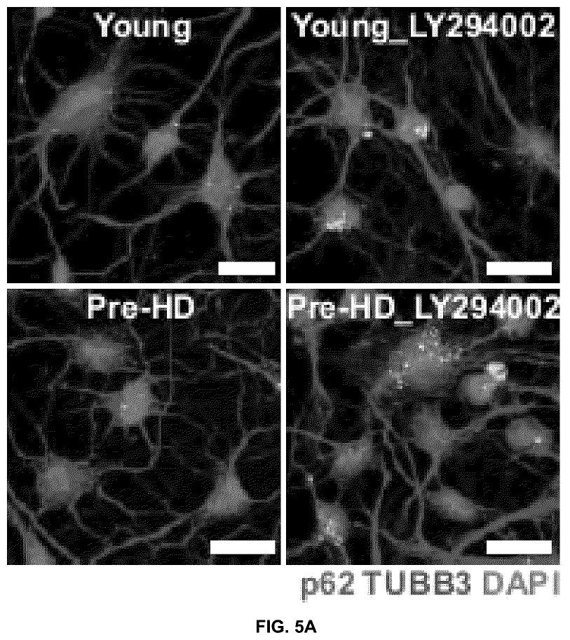

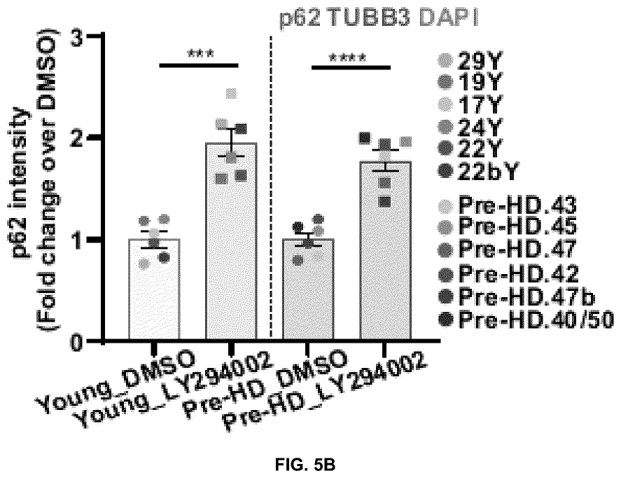

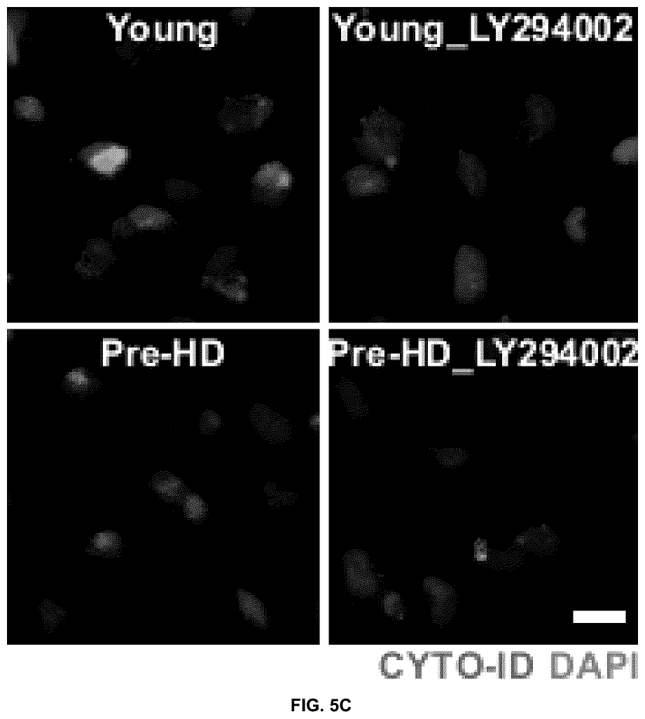

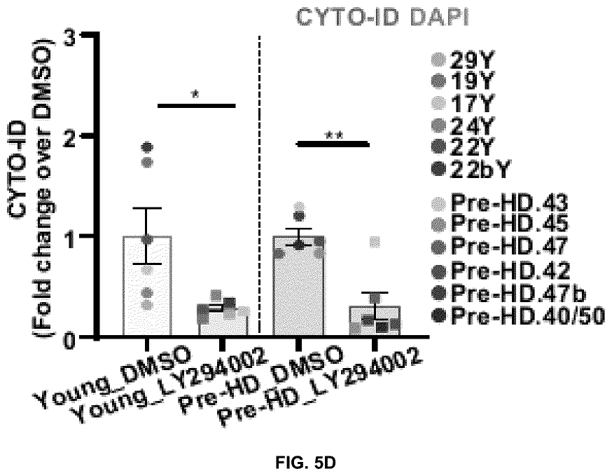

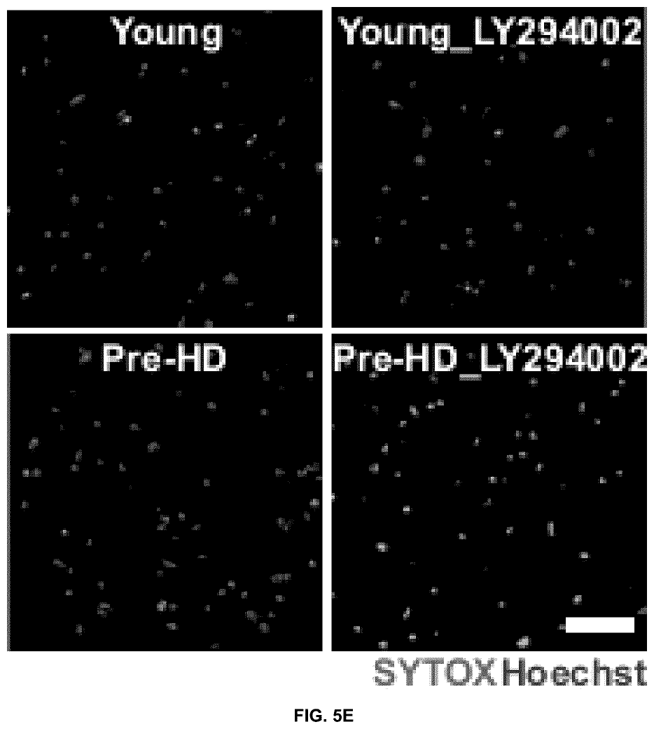

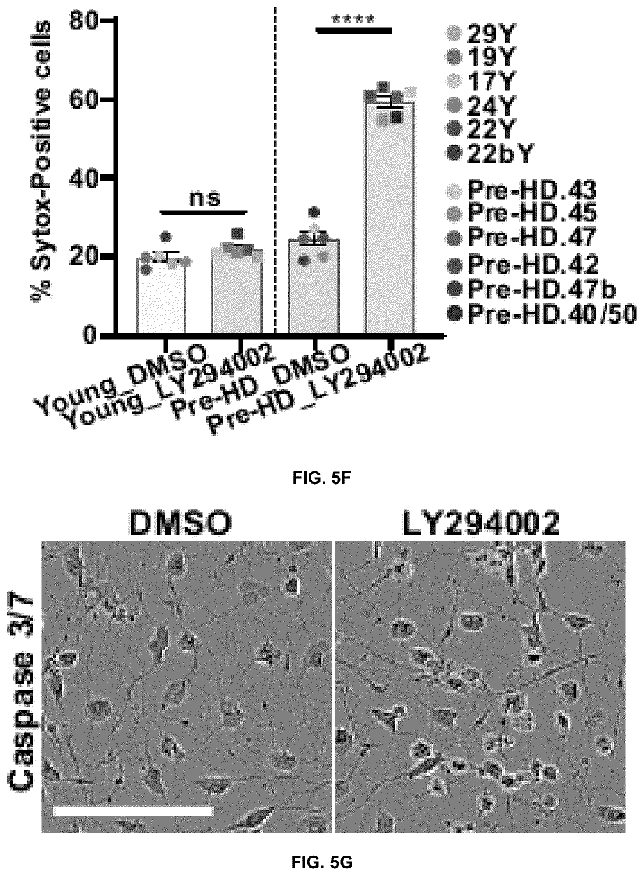

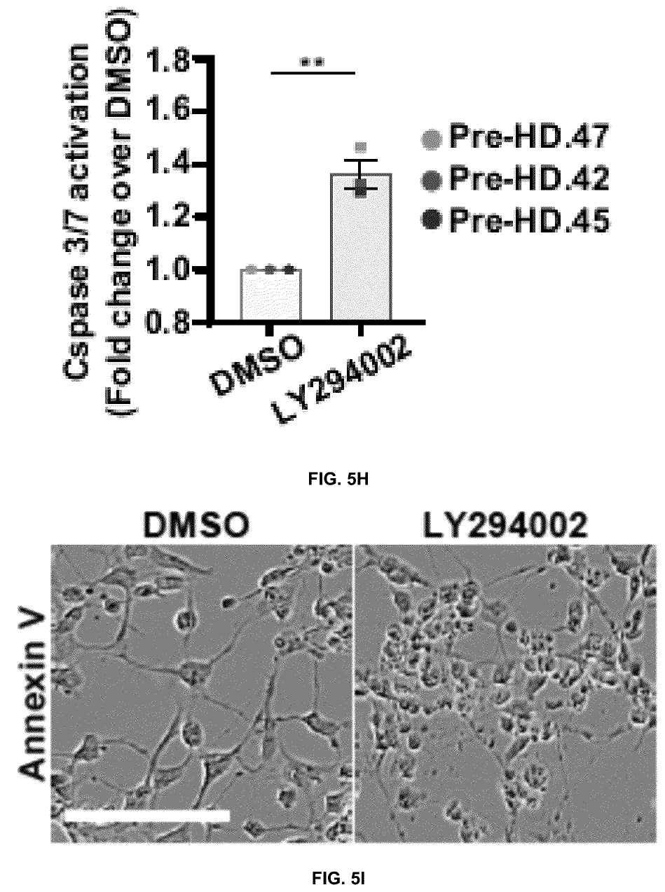

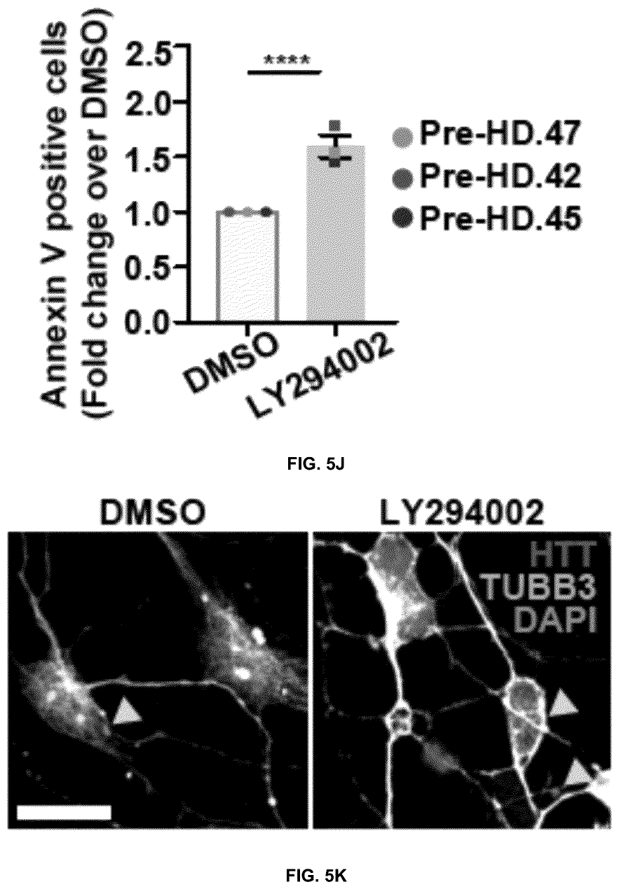

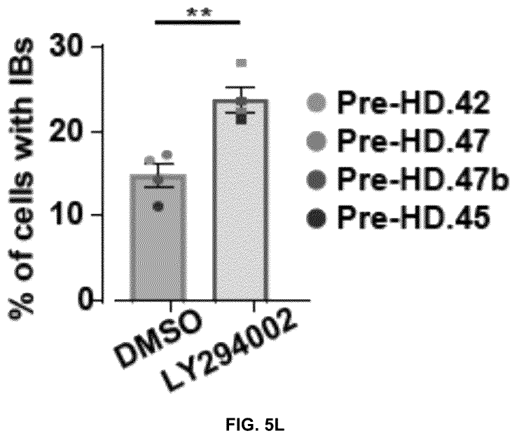

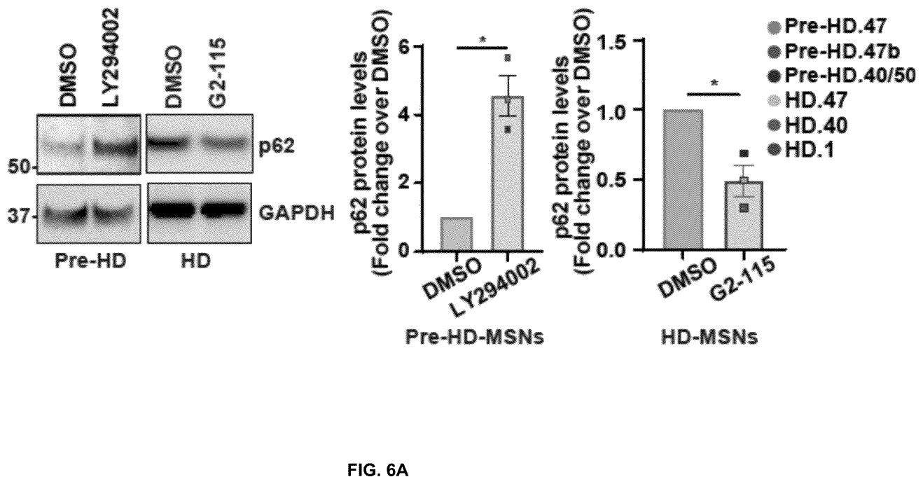

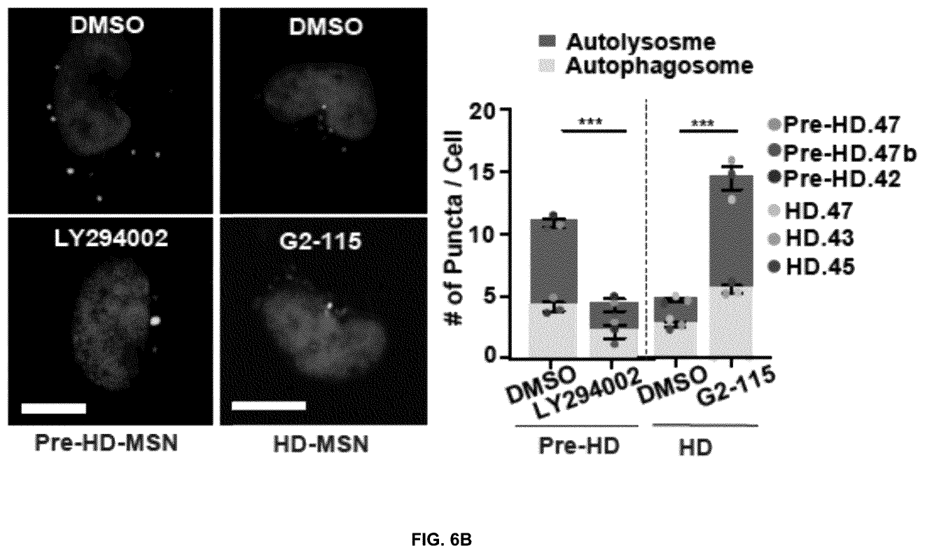

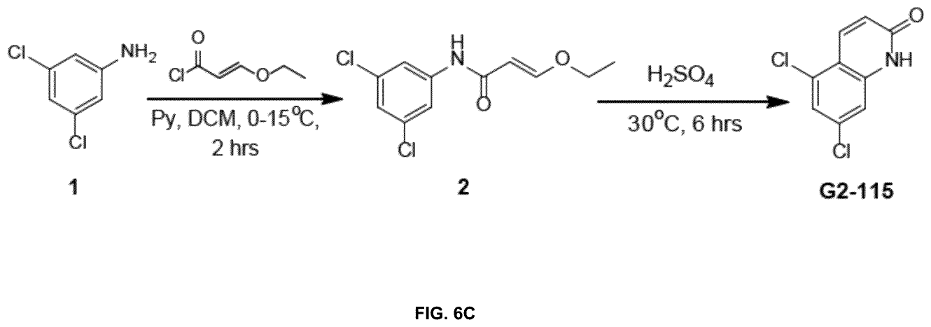

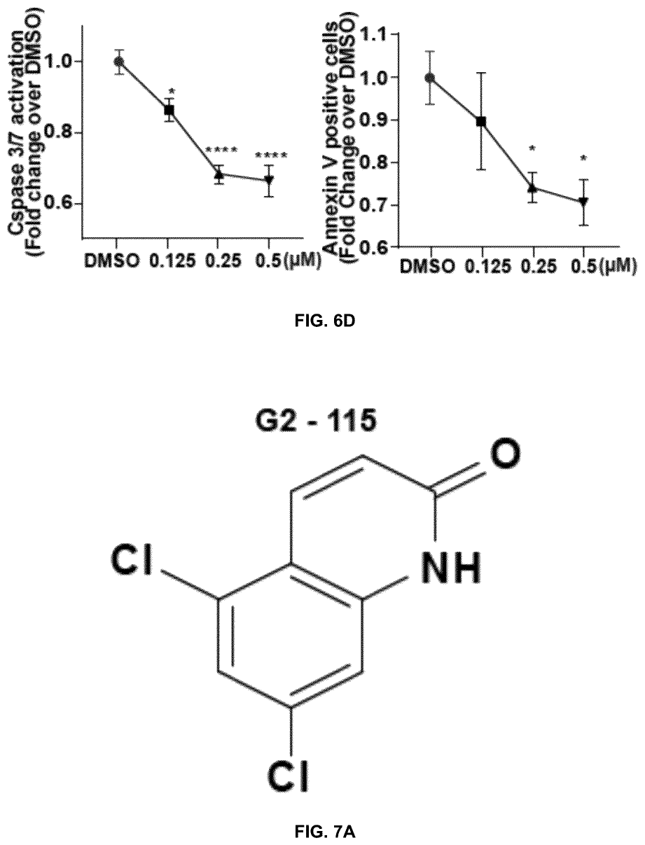

To test the potential link between autophagy reduction and the onset of MSN degeneration, pre-onset HD-MSNs (pre-HD-MSNs, which normally lack the degeneration phenotype compared to HD-MSNs) were treated with LY294002, a compound that inhibits PI3K and autophagy. LY294002 decreased the CYTO-ID signals and increased p62/SQSTM1 expression in young Ctrl-MSNs and pre-HD-MSNs (FIG. and FIG. 6A). Additionally, the average number of autophagosomes and autolysosomes was decreased by LY294002 in pre-HD-MSNs expressing the mCherry-GFP-LC3 reporter (FIG. 6B), confirming the activity of LY294002 in inhibiting autophagy in reprogrammed MSNs. Importantly, LY294002 specifically elevated neuronal cell death of pre-HD-MSNs, as assessed by SYTOX assay at PID30 (FIG. 5E-5F). The detrimental effect of autophagy inhibition was specific for HD patient-derived neurons because treating Ctrl-MSNs from six independent healthy young individuals with LY294002 did not induce neuronal death (FIG. 5E-5F). LY294002 also increased the caspase 3/7 activation signal at PID26 and neuronal cell death at PID30 as assessed by Annexin V staining (FIG. 5G-5J). Furthermore, LY294002 treatment in pre-HD-MSNs significantly increased the number of cells with HTT inclusion bodies compared to the DMSO treatment (FIG. 5K-5L).

### Example 6: Enhancing Autophagy Rescues HD-MSNs from Degeneration

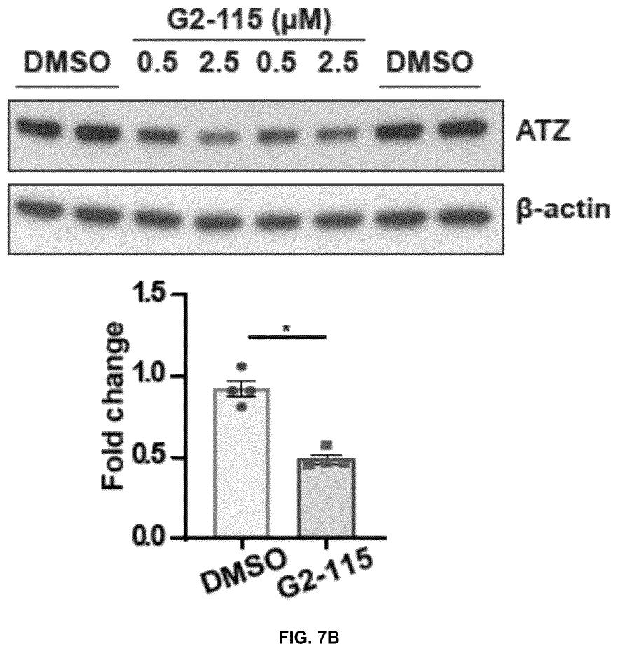

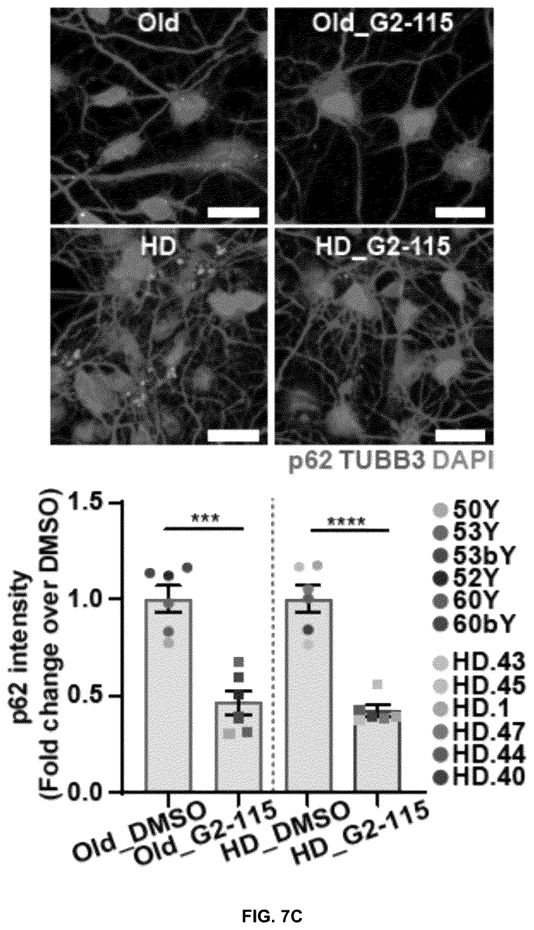

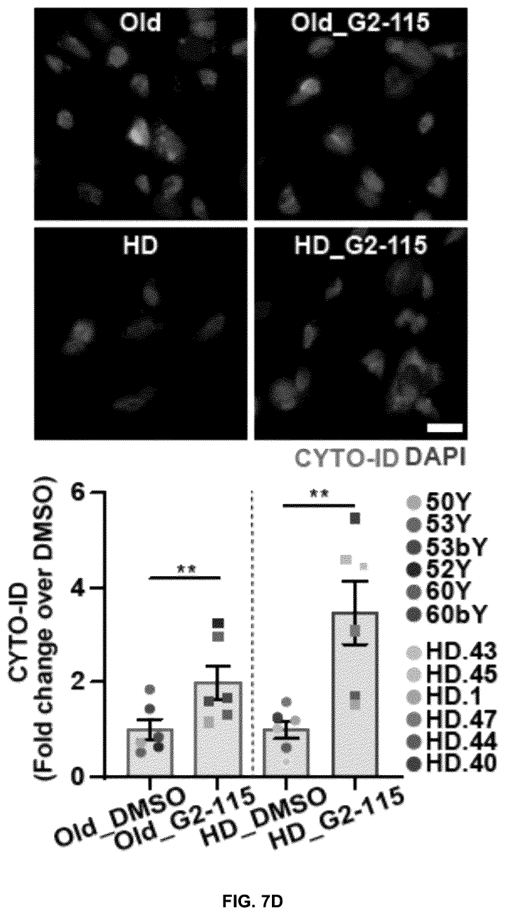

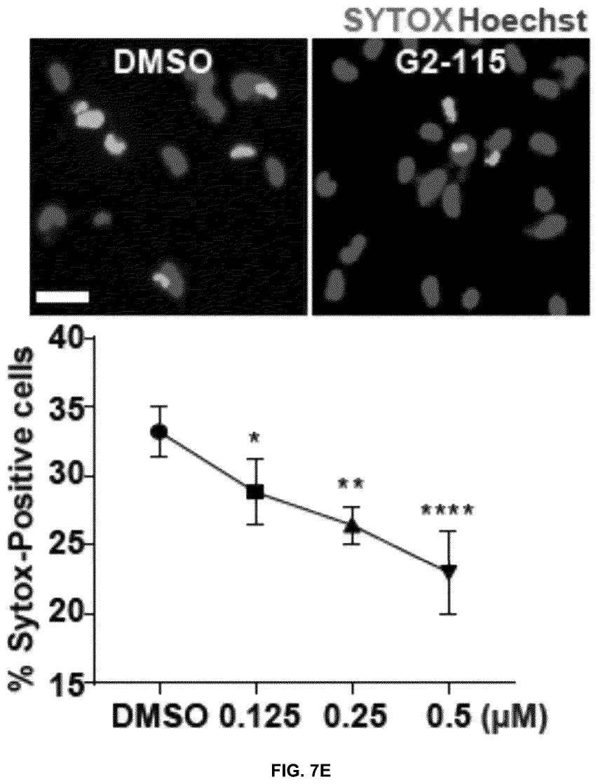

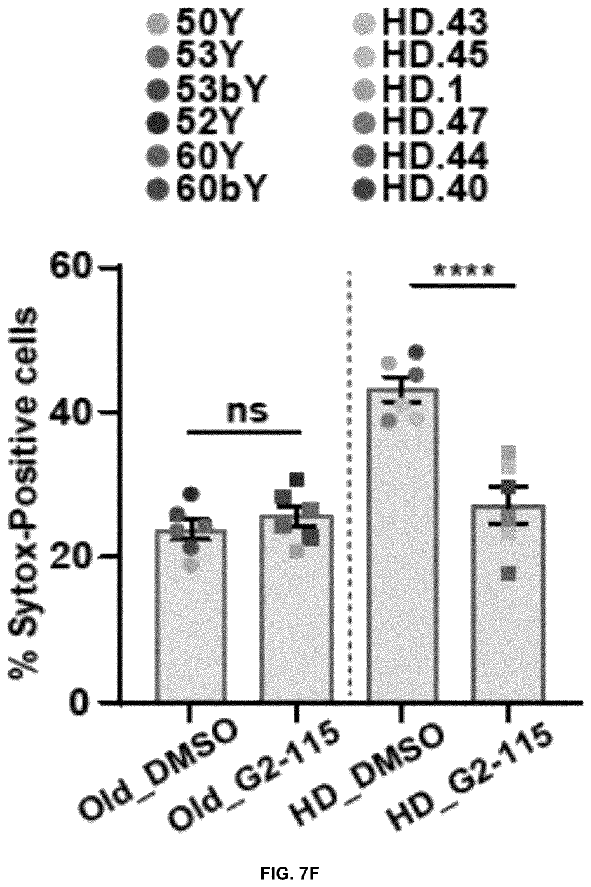

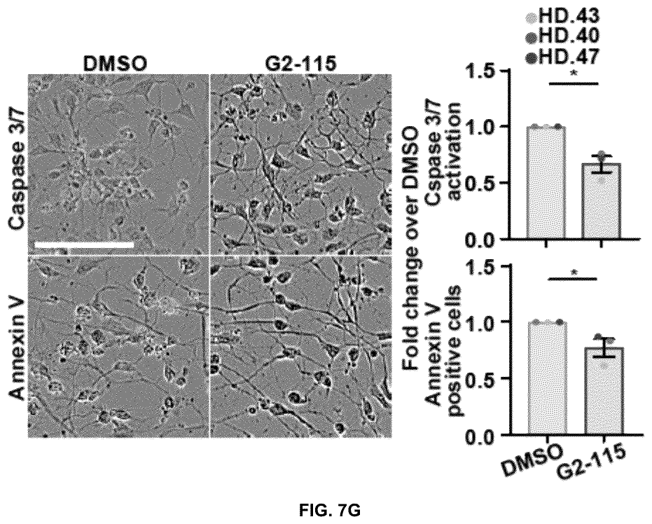

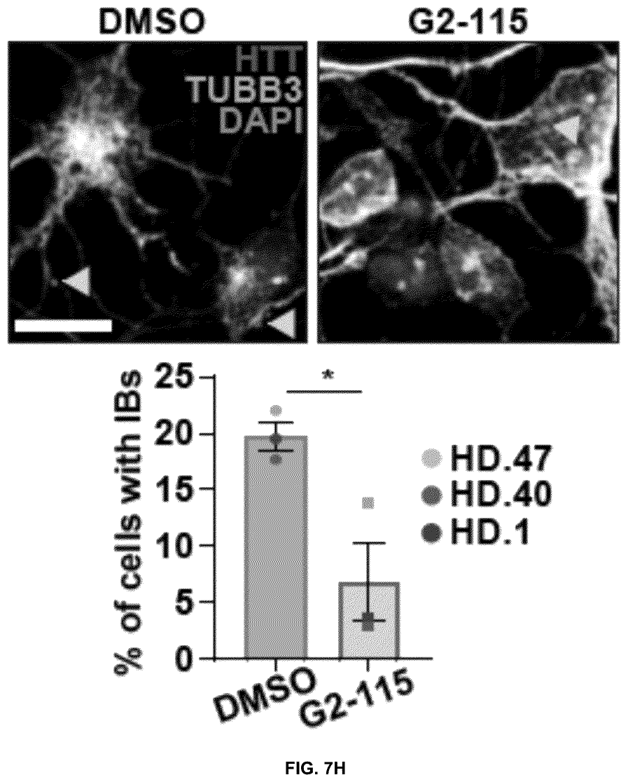

It was further tested if overriding the autophagy deficiency in MSNs derived from symptomatic patients (HD-MSNs) would shift the degeneration state toward pre-HD-MSNs. For this, a new analog of glibenclamide (GLB), a sulfonylurea drug that has been used broadly in clinics as an oral hypoglycemic agent, was developed. A GLB analog, G2, promoted autophagic degradation of misfolded α1-antitrypsin Z variant (ATZ) in mammalian cell models of α1-antitrypsin deficiency (ATD) disorder. The new G2 analog (G2-115), designed to increase the potency of the compound ((FIG. 6C and FIG. 7A), decreased the steady-state levels of ATZ in a HTO/Z cell line model of ATD (FIG. 7B). G2-115 treatment in old Ctrl-MSNs and HD-MSNs increased the number of autophagic vacuoles as measured by CYTO-ID signals and decreased p62/SQSTM1 signal (FIG. 6A and FIG. 7C-7D). Also, G2-115 increased the average number of autophagosomes and autolysosomes in HD-MSNs expressing the mCherry-GFP-LC3 reporter (FIG. 6B), verifying the autophagy-enhancing activity of G2-115 in HD-MSNs. Reprogrammed MSNs were then treated with G2-115 at PID14, a time point when reprogramming cells start adopting the neuronal identity, followed by treatment every four days for 16 days. Importantly, G2-115 promoted HD-MSN survival in a dose-dependent manner reflected by the reduction of SYTOX signal (FIG. 7E). This beneficial effect was consistent in HD-MSNs from six independent patients and specific to HD-MSNs since age-matched Ctrl-MSNs already have lower levels of cell death (FIG. 7F). G2-115 also reduced Caspase 3/7, Annexin V signals and the number of HTT inclusion bodies in HD-MSNs (FIG. 6D and FIG. 7G-7H). These results indicated that in MSNs derived from adult-onset patients with low repeat numbers, enhancing autophagy increased HD-MSNs' ability to clear mHTT aggregation and resilience against neurodegeneration.

### Example 7: Comparative Analysis of Chromatin Accessibilities Between Pre-HD-MSNs and HD-MSNs

To further infer mechanisms underlying differential gene expression, comparative Omni-ATAC-seq was performed to assess differences in chromatin state between pre-HD-MSNs and HD-MSNs. From six independent sex-matched lines of pre- and post-onset HD-MSNs, Omni-ATAC-seq was performed with two or three biological replicates of each MSN line at PID21. Of the total number of 213,045 peaks detected across samples, 28,548 differentially accessible regions (DARs) (adjusted p<0.05, |log2FC|>0.5) were identified between pre-HD-MSNs and HD-MSNs (13% of the total peaks). Of the total DARs, 14,673 DARs corresponded to chromatin regions that became more accessible (opened) and 13,875 DARs to regions that closed more in HD-MSNs. Focusing on DARs±2 kb around the transcription start site (TSS) identified 476 genes with increased and 490 genes with decreased ATAC signals in HD-MSNs compared to pre-HD-MSNs (adjusted p<0.05, |log2FC|>1) (FIG. 8A). Pathway analysis revealed that genes associated with DARs in HD-MSNs were enriched with aging-associated pathways, such as senescence and autophagy, FOXO family signaling, and oxidative stress (FIG. 8B). Examples of those genes include ATG16L1 and ATG10, which are involved in senescence and autophagy, and which show reduced chromatin accessibility in HD-MSNs compared to pre-HD-MSNs (FIG. 8C). Next, integrating the DEG list from the RNA-seq (adjusted p<0.05, |log2FC|>1) with DAR-containing genes at the promoter region (2kb upstream) uncovered 110 upregulated and 253 downregulated genes that coincided with open and closed DARs, respectively (FIG. 9A). Gene ontology analysis revealed that downregulated genes with closed DARs were associated with terms such as apoptosis and protein homeostasis, in contrast to upregulated genes with opened DARs (FIG. 9B-9C).

### Example 8: Identification of miR-29b-3p as a Driver for Autophagy Deficiency in HD-MSNs

Next, how DARs between pre-HD and HD-MSNs may underlie autophagy impairment and neuronal death in HD-MSNs was investigated. The Upstream Regulator Analysis (IPA) was performed for the brown module and lavenderblush3 module downregulated in HD-MSNs and aged Ctrl-MSNs, respectively. Of potential regulators predicted across the modules, transcription factors, SMAD3 and TWIST1, and four miRNAs were uniquely detected in the brown module (FIG. 9D-9E). However, transcript levels SMAD3 and TWIST1 remained unchanged in HD-MSNs according to the results from RNA-seq. As for miRNAs, target predictions by miRTarBase and TargetScan identified miR-29b-3p as the sole miRNA commonly detected across multiple prediction algorithms (FIG. 9F). In genome-wide scanning, DARs proximal to miRNA precursors (±2 kb) between pre-HD-MSNs and HD-MSNs were also located and 163 DARs (adjusted p<0.05, 61 opened and 102 closed) were identified. These DARs corresponded to 29 miRNA precursors with increased DARs and 82 precursors with reduced DARs in HD-MSNs over pre-HD-MSNs. As miRNAs could be upregulated to downregulate genes in the brown module, precursors that contained increased ATAC signals in HD-MSNs over pre-HD-MSNs were examined (FIG. 8D). Of the 29 miRNA precursors containing increased DARs, miR29B1 was predicted to be the most significant regulator of the brown module determined by the relevance score (FIG. 8D). MiR29B1, which showed increased chromatin accessibility in HD-MSNs over pre-HD-MSNs (FIG. 8E), is a host gene of miR-29b-3p, a mature miRNA predicted as an upstream regulator of the brown module (FIG. 9E-9F). Finally, pathway analysis of miR-29b-3p target genes in the brown module showed association with senescence and autophagy (FIG. 9G).

### Example 9: Age-Associated Expression of miR-29b-3p in HD

It was further tested whether miR-29b-3p, the mature miRNA from miR29B1, would be expressed higher in HD-MSNs compared to pre-HD-MSNs. miR-29b-3p expression was significantly increased in HD-MSNs over pre-HD-MSNs as measured by qPCR (FIG. 8F). Interestingly, there was a mild increase in miR-29b-3p in old Ctrl-MSNs over young Ctrl-MSNs (FIG. 8G). However, the extent of miR-29b-3p upregulation was significantly more pronounced in HD-MSNs over pre-HD-MSNs (210% increase), compared to old Ctrl-MSNs over young Ctrl-MSNs (20% increase) (FIG. 8H). Of note, the age-associated increase in miR-29b-3p was also observed in the striatum of human brain samples from six elderly, cognitively normal individuals (83, 84, 85, 87, and 91 years of age) over the striatum of six young healthy individuals (8, 9, 11, and 19 years of age) (FIG. 8I). Also, miR-29b-3p levels were compared in the basal ganglia sections from five independent HD patients and five healthy individuals and was found that HD samples had higher levels of miR-29b-3p (FIG. 8J). In summary, miR-29b-3p was identified as a miRNA whose expression increased with aging, which becomes more exaggerated in HD-MSNs over pre-HD-MSNs with alterations in the chromatin accessibility.

### Example 10: miR-29b-3p Inhibition Enhances Autophagy and Rescues HD-MSNs from Degeneration

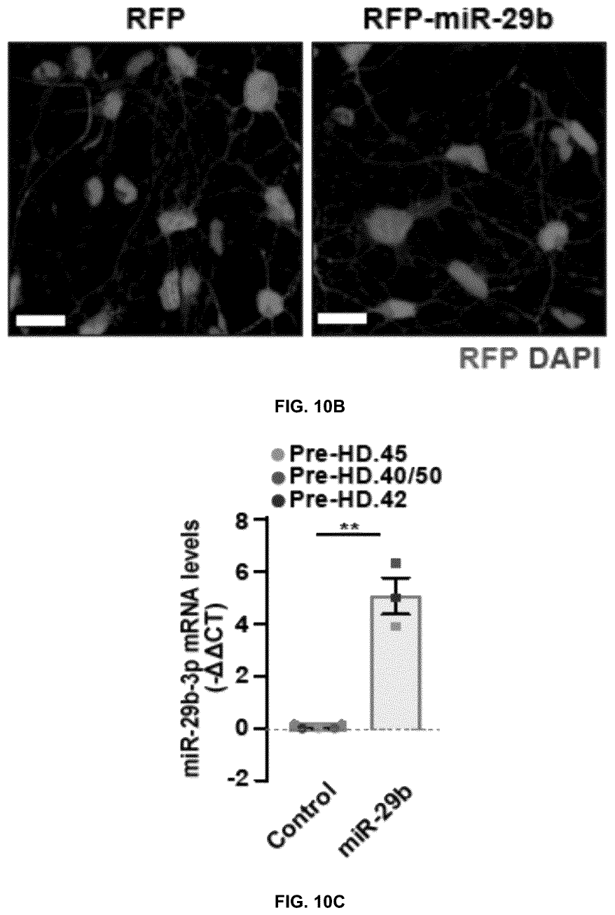

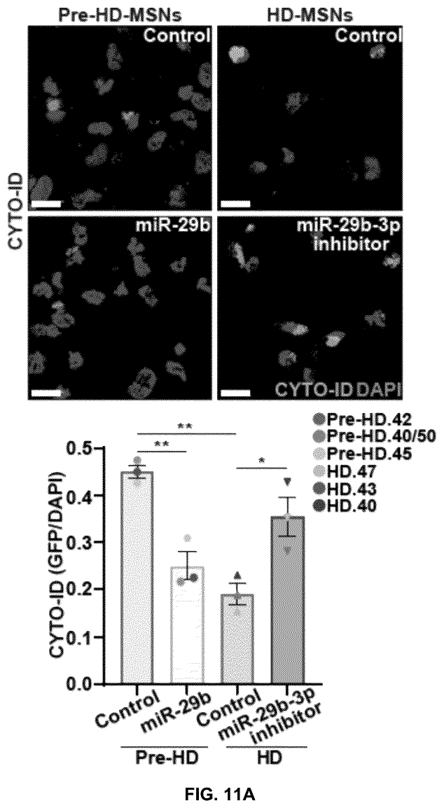

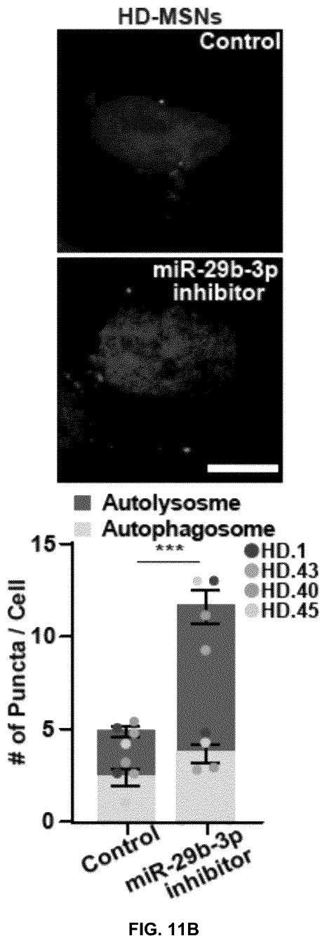

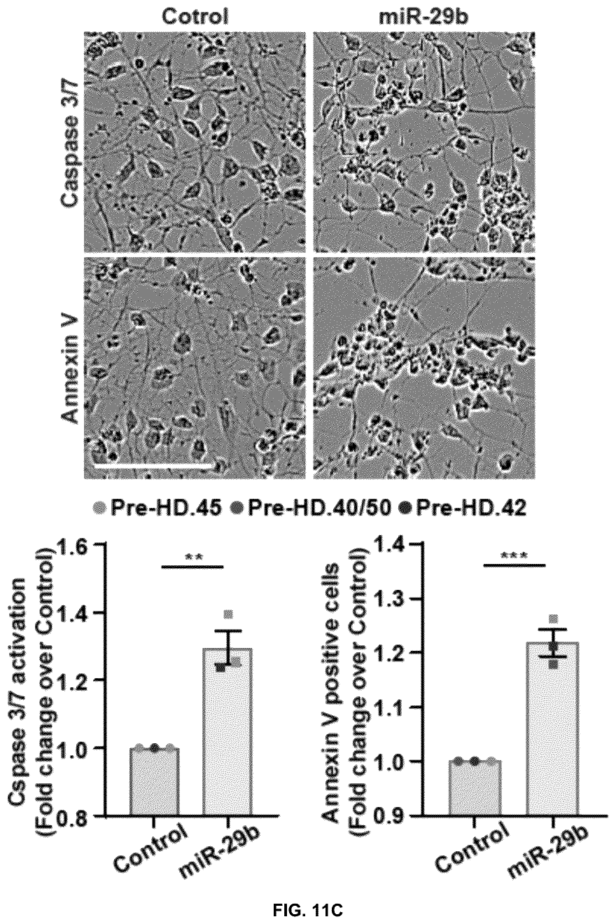

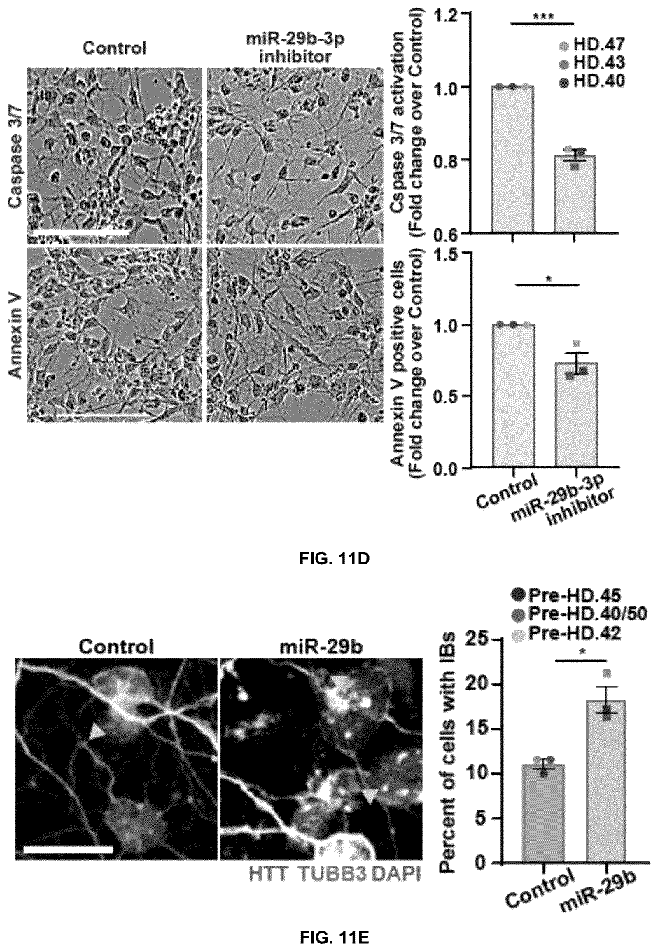

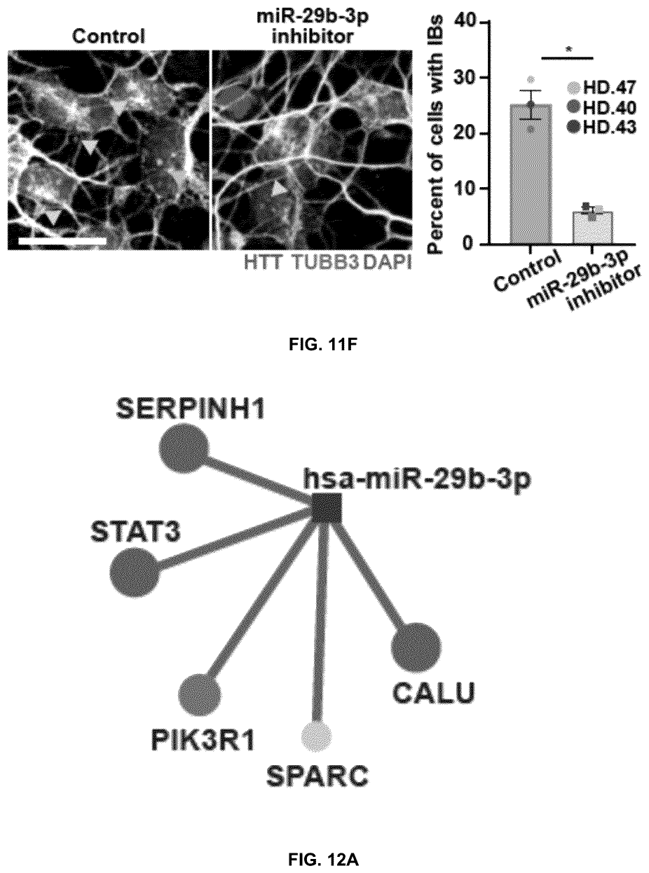

To investigate the involvement of miR-29b-3p in autophagy dysfunction, genetic perturbation experiments were undertaken by either reducing or increasing miR-29b-3p expression. It was found that the antisense power inhibitor of miR-29b-3p (Qiagen) was sufficient to reduce miR-29b-3p substantially in reprogrammed MSNs (FIG. 10A). For overexpression, a lentivirus vector was constructed to overexpress the miR-29b precursor (FIG. 10B-10C). Overexpressing miR-29b-3p in pre-HD-MSNs decreased autophagy activity, whereas reducing miR-29b-3p in HD-MSNs increased autophagy activity compared to the scrambled control, as revealed by an increase in the CYTO-ID signal (FIG. 11A) and the average number of both autophagosomes and autolysosome signals from the tandem mCherry-GFP-tagged LC3 reporter (FIG. 11B). These results demonstrated that the autophagic state in HD-MSNs can be modified by altering miR-29b-3p levels, either pushing it towards the pre-HD state by miR-29-3p inhibition in HD-MSNs or towards HD-MSN state by overexpressing in pre-HD-MSNs. We then measured apoptotic signals in pre-HD- and HD-MSNs while altering the miR-29b-3p level. Overexpressing miR-29b-3p in pre-HD-MSNs significantly increased Caspase 3/7 and Annexin V signals compared to the control (RFP expression only), whereas inhibiting miR-29b-3p decreased Caspase 3/7 and AnnexinV signals in HD-MSNs (FIG. 11C-11D). Because mHTT aggregation has been linked to autophagy51, it was examined if altering miR-29b-3p would influence the amount of mHTT aggregation. Overexpressing miR-29b in three independent pre-HD MSN samples significantly increased mHTT aggregation compared to the control (FIG. 11E), whereas treating HD-MSNs with the miR-29b-3p inhibitor significantly reduced the number of cells with mHTT aggregation (FIG. 11F). These results collectively demonstrated that reducing miR-29b-3p promoted autophagy and alleviated HD-MSN from degeneration.

### Example 11: STAT3 is Directly Targeted by miR-29b-3p Impairing Autophagy in HD-MSNs

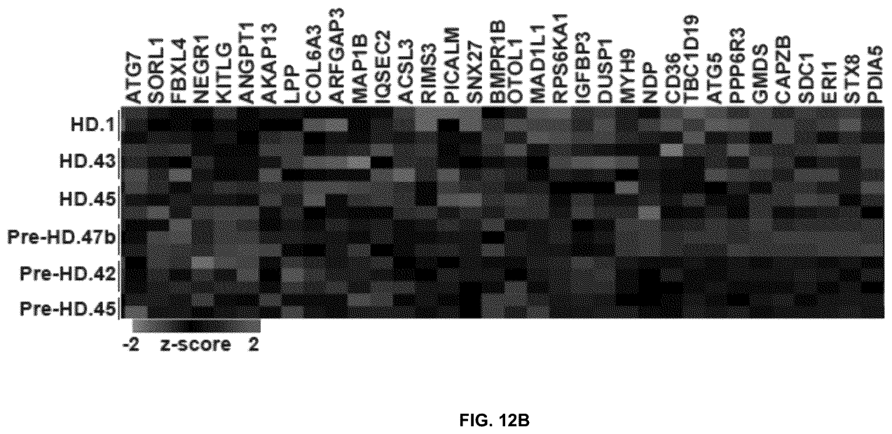

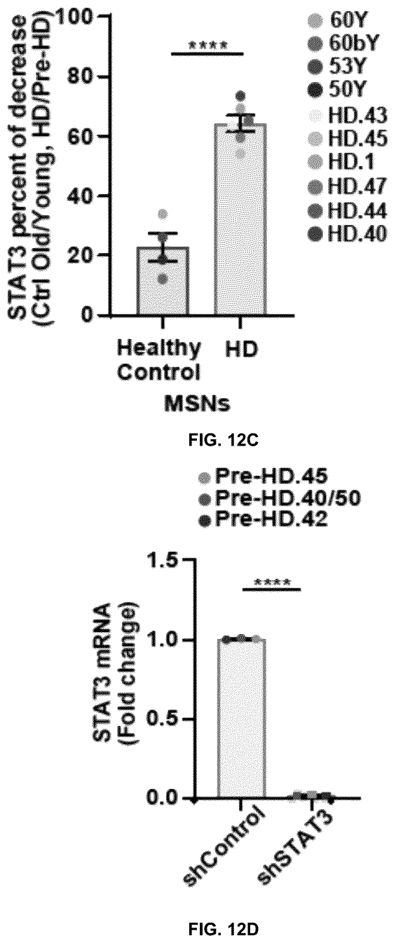

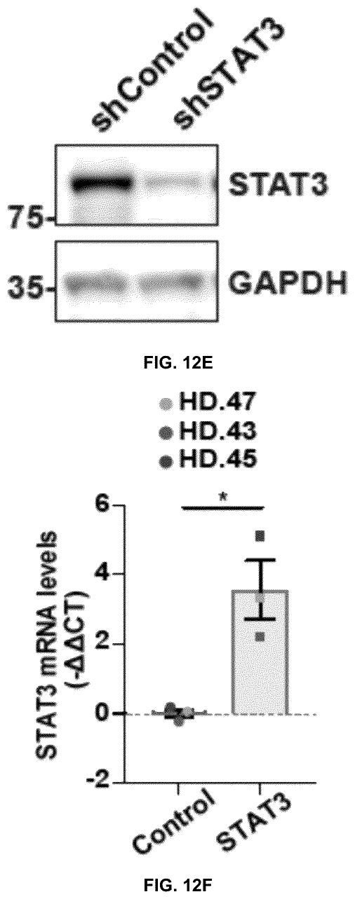

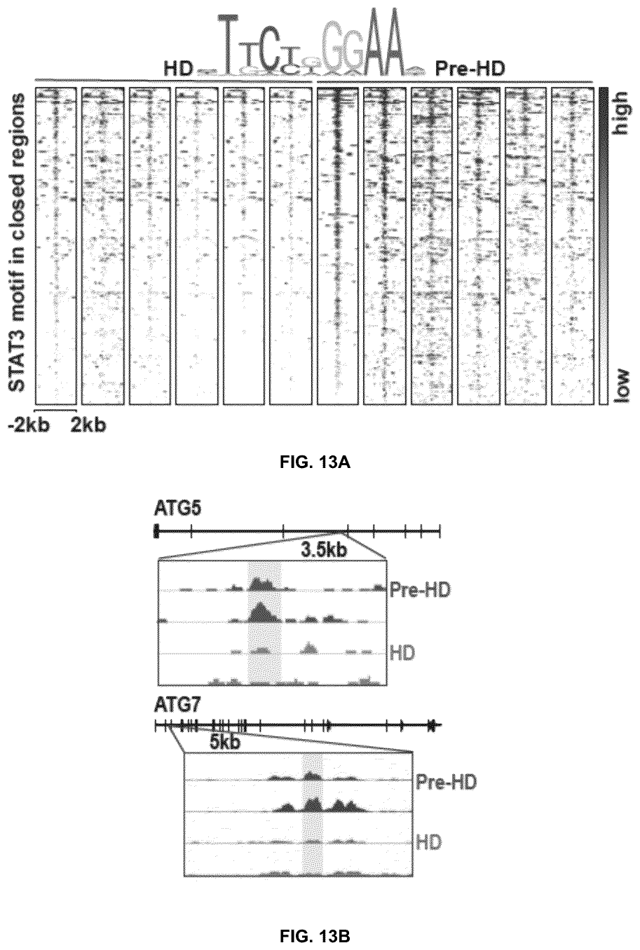

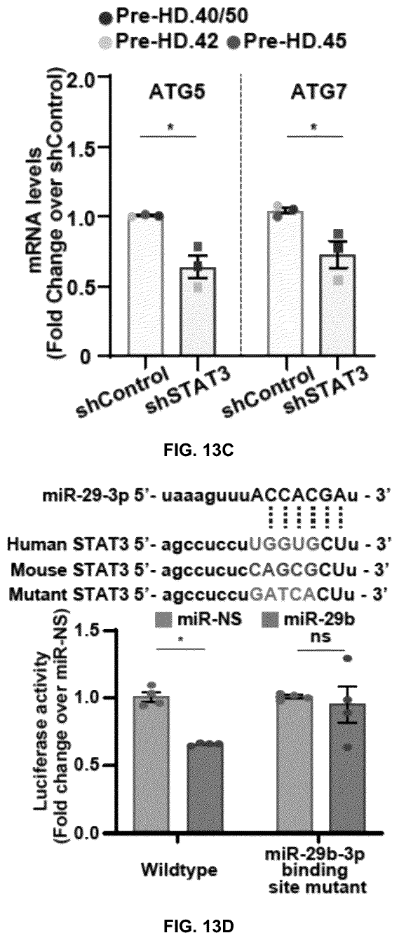

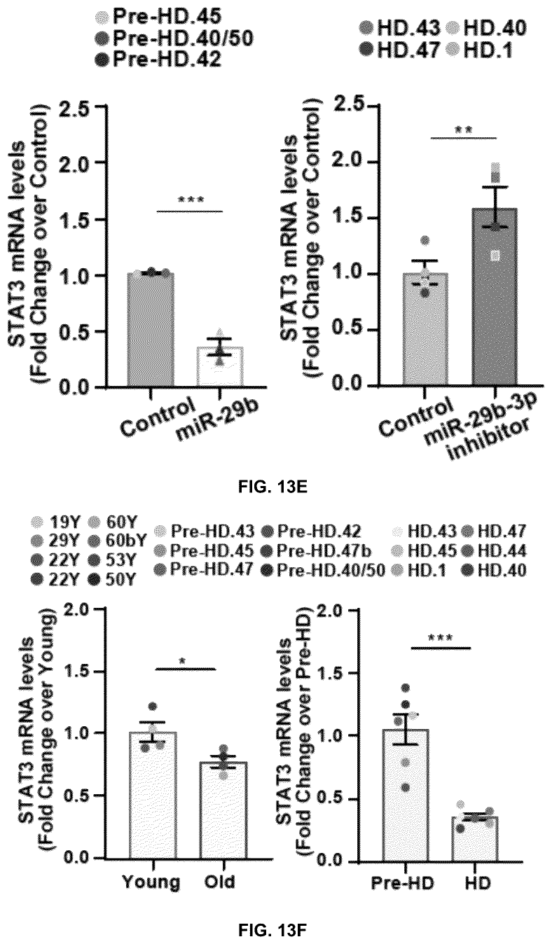

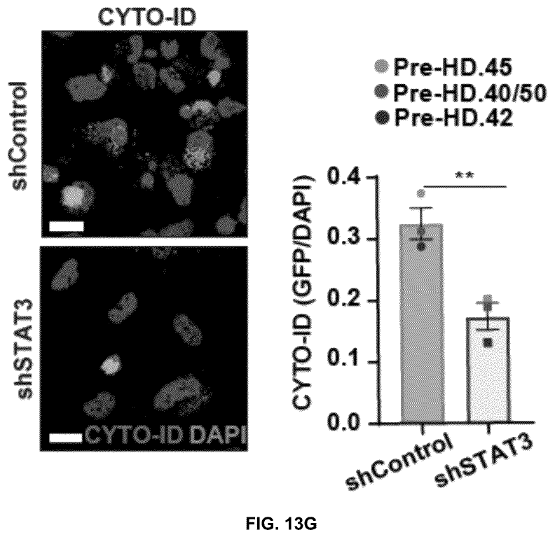

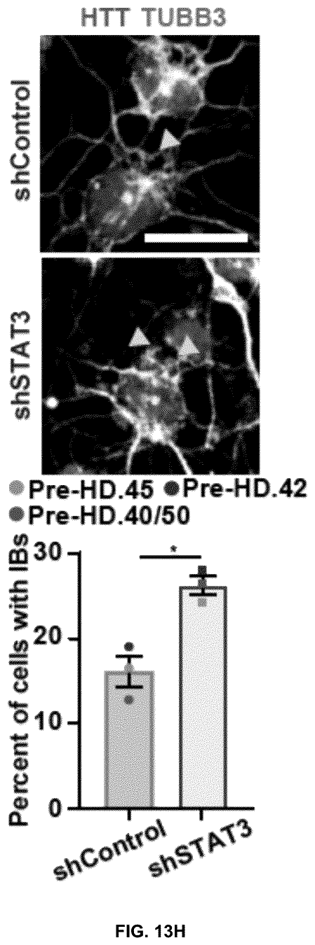

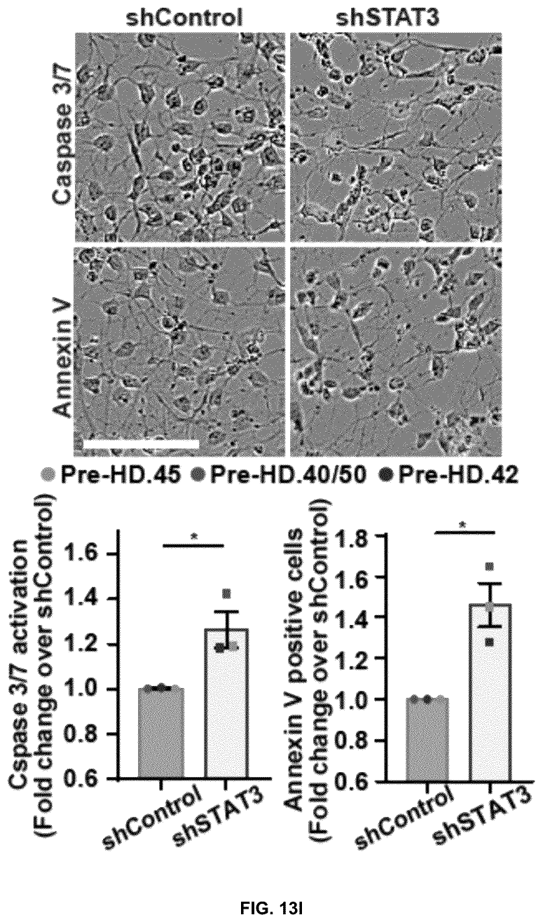

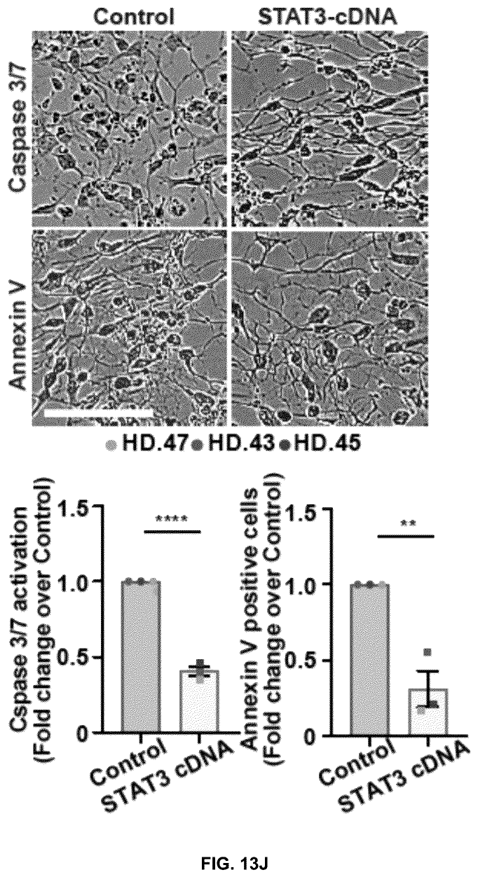

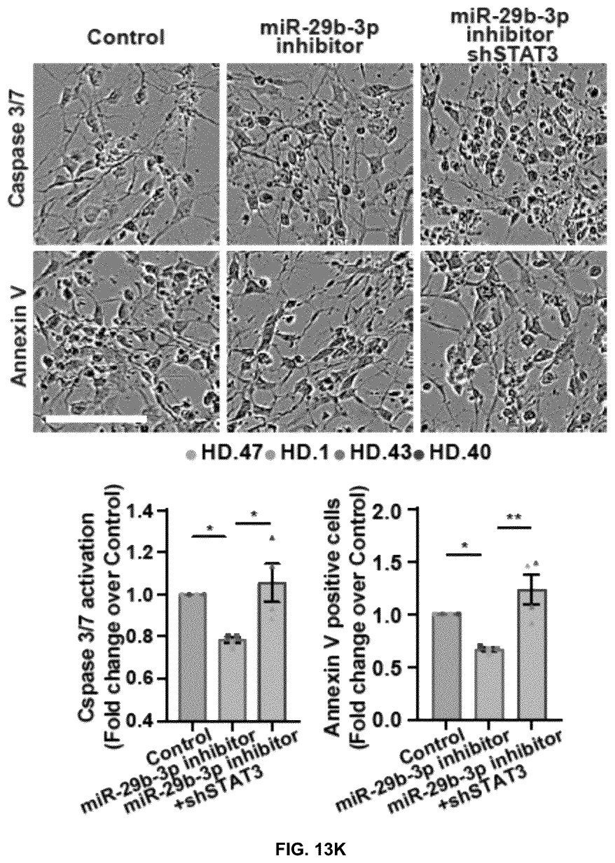

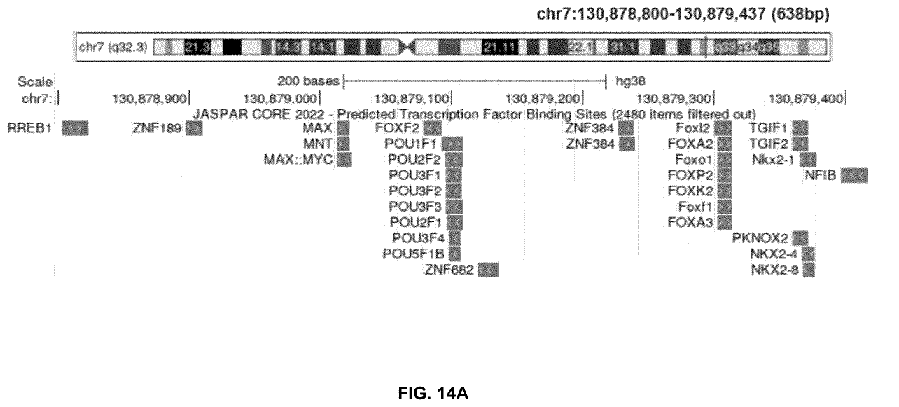

Critical target of miR-29b-3p responsible for autophagy reduction in HD-MSNs was further examined. Among the predicted target genes of miR-29b-3p in the brown module (FIG. 12A), STAT3 was focused on for several reasons. First, searching for common sequence motifs within the chromatin regions significantly closed in HD-MSNs compared to pre-HD-MSNs identified a significantly enriched consensus sequence motif corresponding to the binding site of STAT3 (JASPER transcription factor database) (FIG. 13A). Second, STAT3 was identified as one of the autophagy-related hub genes in the brown module (FIG. 4D). Third, genes that contained STAT3 binding site in the closed DARs in HD-MSNs were enriched with genes associated with autophagy (FIG. 12B); among 191 genes that contained STAT3 binding sites associated with closed DARs in HD-MSNs, 23% of them were linked to autophagy (adjusted p<0.05, log2FC≤−1), including ATG5 and ATG7 (FIG. 13B). Knocking down STAT3 in pre-HD-MSNs significantly decreased the expression of ATG5 and ATG7 over the shControl (FIG. 13C). Moreover, examining sequences within the 3′UTR of STAT3 revealed a seed-match sequence for miR-29b-3p, which appeared to be human-specific and not conserved in mice (FIG. 13D). It was found that STAT3 was a direct target of miR-29b-3p as confirmed by a luciferase assay in HEK293Le cells in which miR-29b-3p expression effectively targeted the 3′UTR of STAT3 and reduced the luciferase activity, whereas point mutations in the seed-match sequence in STAT3 3′UTR abolished the targeting activity (FIG. 13D). Overexpressing miR-29b-3p in pre-HD-MSNs led to decreased expression of STAT3, whereas treating HD-MSNs with miR-29b-3p inhibitor increased STAT3 expression (FIG. 13E), further supporting the notion that miR-29b-3p regulated STAT3 as a direct target. Other targets of miR-29b-3p included FOS and SIRT1. However, these genes were not part of the brown module genes and differentially expressed genes between pre-HD-MSNs and HD-MSNs. In addition, while STAT3 mRNA was found to be lower in old Ctrl-MSNs over young Ctrl-MSNs, the degree of reduction was much more pronounced between pre-HD-MSNs and HD-MSNs (20% versus 60%) (FIG. 12C and FIG. 13F).

Next, it was investigated if STAT3 was involved in the regulation of autophagy activity in patient-derived MSNs. First, directly knocking down STAT3 by shRNA in pre-HD-MSNs (FIG. 12D-12E) decreased the autophagy activity (FIG. 13G), increased mHTT aggregation compared to the control (FIG. 13H), and increased apoptosis of pre-HD-MSNs (FIG. 13I), whereas overexpression of STAT3 cDNA in HD-MSNs rescued cells from neuronal death (FIG. 13J). While treating HD-MSNs with miR-29b-3p inhibitor led to decreased neuronal death, this effect was reversed by knocking down STAT3 in the presence of miR-29b-3p inhibitor (FIG. 13K). Overall, the results highlight the interaction between miR-29b-3p and STAT3 as an integral component driving HD-MSN degeneration.

### SUMMARY

HD is an adult-onset disorder in most HD cases. Yet, age-associated pathways that contribute to the onset of HD pathology in patients have remained largely elusive. Elucidating such pathways, especially in the spectrum of human lifespan, has been a challenging task due to the inability to model the progression of HD pathology with patient neurons. The disclosure herein used directly reprogrammed MSNs from pre-symptomatic and symptomatic stages of HD to understand differences in cell-intrinsic properties that render HD-MSNs more vulnerable to degeneration than their pre-symptomatic counterparts. Given that the m icroRNA-mediated neuronal reprogramming occurs through step-wise processes of fibroblast fate erasure and adoption of the neuronal identity, detecting differences in genetic networks as cells acquire MSN identity offers an experimental means to dissect gene expression and chromatin landscape changes in directly reprogrammed MSNs from different disease stages.

The identification of reduced autophagy activities in HD-MSNs (from symptomatic patients) associated with transcriptome and chromatin changes allowed to reveal the miR-29b-3p-STAT3 axis as a driver of HD-MSN degeneration linked to reduced autophagy. The results disclosed herein demonstrated the feasibility of enhancing autophagy and increasing MSN resilience against the mHTT-induced toxicity either by repressing miR-29b-3p or through pharmacological means. Of the target genes of miR-29b-3p, STAT3 was identified as a direct target whose reduced expression leads to chromatin closure for genes important for autophagy such as ATG5 and ATG7. Interestingly, the 3′UTR of STAT3 contains a seed-match sequence (UGGUGCU) for miR-29b-3p, which appears to be primarily in humans. The findings disclosed herein demonstrated the importance of miRNA-target interaction that may be unique to humans and lend further support for the use of patient cell-based modeling platforms.

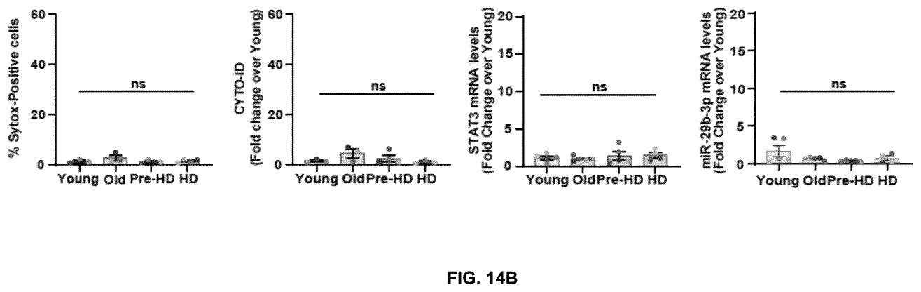

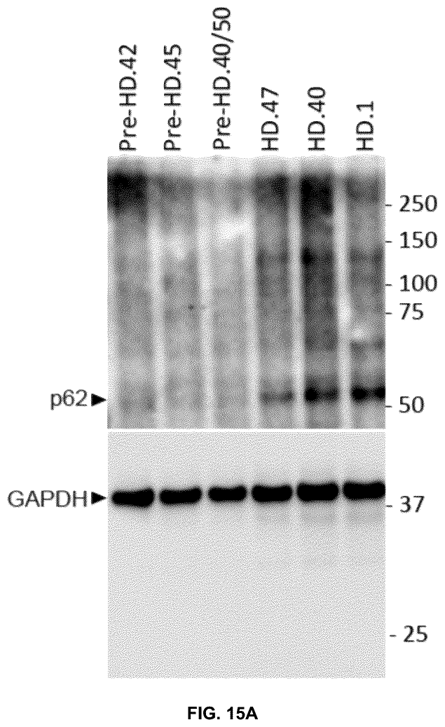

Because miRNAs usually target not only a single gene, but multiple components of related pathways, the discovery of miRNAs as important modulators for disease pathology has expanded therapeutic opportunities for oligonucleotides. Based on the results shown for HD neurons, miRNA antagonist approaches should also be considered to mitigate the effect of the age-associated increase in miR-29b-3p, as detected in both reprogrammed MSNs and aged human brain samples. Interestingly, the DAR proximal to miR29B1 was predicted to contain binding motifs for FOXO1 and FOXA3 (FIG. 14A).

Activation of autophagy can successfully lower mHTT aggregations in mouse and human neuron models of HD. However, how the impairment of autophagy arises during the adult-onset of HD in humans has remained poorly understood. The current disclosure, provided herein is evidence that in the context of human neurons, STAT3 reduction, due to miR-29b-3p, played a critical role during the degeneration of HD patient-derived MSN. Further investigations into the use of autophagy enhancer compounds, such as the G2 analog or antisense oligo against miR-29b-3p, may eventually offer an effective therapeutic angle that could increase the resilience of MSNs against neurodegeneration in HD. Although the molecular target of drug action for the G2 analog is not known, the data provided herein is proof-in-principle that the age-associated decline of autophagy in patients' MSNs can be countered and alleviated by pharmacological interventions.

Further provided herein, are isolated genetic pathways and differential neurodegenerative states that correlated with different stages of HD by focusing on phenotypes consistently manifested in MSNs reprogrammed from multiple patients' samples. Interestingly, HD-specific phenotypes, such as increased cell death and decreased autophagy activity, were not detected in fibroblasts of post-onset, pre-onset HD patients, and healthy control samples. Also, the expression levels of miR-29b-3p and STAT3 were not different between healthy control, pre-HD, and HD fibroblasts, demonstrating the requirement of the directly reprogrammed MSN identity to reveal cellular pathologies and genetic networks underlying HD (FIG. 14B). It is likely that future studies implementing direct neuronal reprogramming should benefit from the rapidly advancing genome-editing technologies that can further advance the modeling and defining pathogenic mechanisms of late-onset neurodegenerative disorders.

In summary, striatal medium spiny neurons (MSNs) directly reprogrammed from fibroblasts of age-matched healthy individuals and HD patients were implemented to model the age-dependent onset of HD pathology. It was found that neuronal death was selectively pronounced in reprogrammed MSNs from symptomatic HD patients (HD-MSNs) compared to MSNs derived from younger, pre-symptomatic patients (pre-HD-MSNs) and control MSNs from age-matched healthy individuals. Comparative transcriptome chromatin analyses between HD-MSNs and pre-HD-MSNs revealed age-associated alteration in chromatin accessibilities, and identified miR-29b-3p, whose age-associated upregulation promotes HD-MSN degeneration by impairing autophagic function through human-specific targeting of STAT3 3′UTR. The autophagy deficiency in HD-MSNs can be overcome chemically or genetically by a glibenclamide analog, G2 or inhibiting miR-29b-3p, leading to the reduction of mutant HTT aggregation and protection of HD-MSNs from neuronal death. These results demonstrated miRNA upregulation with aging in HD as a detrimental process driving MSN degeneration and provides a potential approach for enhancing autophagy and resilience of HD-MSNs.

What is claimed is:

1. A synthetic antisense RNA (or RNAs) for targeting miR-29b-3p for treatment of HD.

2. A composition comprising a therapeutic amount of one or more antisense RNA of claim 1 for treatment of Huntington's Disease.

3. The composition of claim 2, further comprising a pharmaceutically acceptable carrier.

4. The composition of claim 2, further comprising glibenclamide or a glibenclamide analog.

5. The composition of claim 4, wherein the glibenclamide analog is G2-115.

6. A method of treatment of Huntington's disease in a subject in need thereof, the method comprising administering an miR-29b-3p inhibitor to the subject, wherein administration results in enhanced neuronal autophagy.

7. The method of claim 4, wherein the miR-29b-3p inhibitor is an antisense RNA targeting miR-29b-3p.

8. The method of claim 6, further comprising administering a glibenclamide analog to the subject.

9. The method of claim 8, wherein the glibenclamide analog is G2-115.

10. The method of claim 6, wherein neuronal apoptosis is reduced.

11. The method of claim 6, wherein the miR-29b-3p inhibitor augments STAT3.

12. The method of claim 6, wherein the Huntington's Disease is late Huntington's Disease.

13. A method of treatment of Huntington's disease in a subject in need thereof, the method comprising administration of a glibenclamide analog to the subject, wherein administration results in enhanced neuronal autophagy.

14. The method of claim 13, wherein the glibbenclamide analog is G2-115.

15. The method of claim 13, wherein apoptosis is reduced.

16. The method of claim 13, further comprising administering an miR-29b-3p inhibitor to the subject.

17. The method of claim 16, wherein the miR-29b-3p inhibitor is an antisense RNA targeting miR-29b-3p.

18. The method of claim 13, wherein the miR-29b-3p inhibitor augments STAT3.

19. The method of claim 13, wherein the Huntington's Disease is late Huntington's Disease.
